(19) United States
(12) Patent Application Publication
(10) Pub. No.: US 2014/0149047 A1
(43) Pub. Date: May 29, 2014

(54) Dynamic and Differential Analysis

(71) Applicant: Soar BioDynamics, Ltd., Incline Village, NV (US)

(72) Inventor: Thomas NEVILLE, Incline Village, NV (US)

(73) Assignee: Soar BioDynamics, Ltd., Incline Village, NV (US)

(21) Appl. No.: 14/154,301

(22) Filed: Jan. 14, 2014

Related U.S. Application Data
(63) Continuation of application No. 13/442,648, filed on Apr. 9, 2012.
(60) Provisional application No. 61/472,975, filed on Apr. 7, 2011.

(51) Int. Cl. G01N 33/574

(52) U.S. Cl. CPC G01N 33/57434
USPC 702/19

(57) ABSTRACT

The present invention provides methods to improve upon prostate cancer screening, thereby saving lives and reducing morbidities of unwarranted biopsies and over-treatment. The methods use a systematic analysis of the growth rate of PSA from cancer and PSA variation and the way in which they might be used to distinguish high-risk cancers from no cancer. Approaches include Dynamic-Differential Strategy, Dynamic-Differential Analysis, and Dynamic Analysis.

### CROSS-REFERENCE

This application is a continuation of U.S. patent application Ser. No. 13/442,648, filed Apr. 9, 2012 and entitled “Dynamic and Differential Analysis,” to which we claim priority under 35 U.S.C. §120, and which claims the benefit of U.S. Provisional Application No. 61/472,975, filed Apr. 7, 2011 and entitled “Dynamic and Differential Analysis.” These two applications are fully incorporated herein by reference.

### BACKGROUND OF THE INVENTION

Current prostate cancer screening methods typically use an initial screening step that measures one or more biomarkers, such as that of prostate specific antigen (PSA), Free PSA (FPSA), or the ratio of FPSA to PSA (FPSA %). Subjects with a PSA value above a certain threshold, typically 4.0 ng/mL, are then recommended for further screening using prostate biopsies. Biopsies are an invasive procedure, and are associated with a variety of risks and side effects, such as: pain and ongoing discomfort, bleeding and blood in semen, urinary tract infection and problems urinating. In contrast, benign conditions are typically diagnosed by symptom, e.g.: prostatitis symptoms include frequent or urgent urination and pain, burning or difficulty urinating, and benign prostatic hyperplasia (BPH) symptoms often relate to problems with bladder emptying (hesitancy, weak flow, etc.) and bladder storage (waking at night to urinate, frequent urination, etc.).

There are three major problems with current prostate cancer screening.

Excessive Recurrence and Death—Despite current recommended screening methods, prostate cancer remains a leading cause of male death from cancer, and recurrence after treatment can be 30% or more in screening populations, such as in patients affiliated with the Veteran's Administration. Late detection and treatment is often a cause of excessive recurrence. Another cause is the inability to identify High Gleason cancers. Gleason score is a pathologist's estimate of cancer aggressiveness. High Gleason cancers are responsible for a high proportion of cancer recurrence and subsequent death.

Unwarranted Biopsies—Roughly 75% of U.S. biopsies do not find cancer because elevated PSA is not specific to prostate cancer. Benign prostate hyperplasia (BPH) and prostatitis (inflammation and/or infection) can increase PSA values. The CDC has found that 13% of men over 40 have a PSA in excess of 2.5 ng/mL, while 6% of men over 40 have a PSA in excess of 4.0 ng/mL. Only a small percentage of men with PSA values this high have PSAs that are caused primarily by cancer, resulting in a high false positive rate. Further, a single PSA test result has poor specificity (especially for values as low as 2.5), so the threshold to trigger further screening is set high to increase specificity. However, this comes at the cost of late detection and a higher risk of recurrence for a significant percent of patients.

Over-Treatment of Indolent Cancer—Many doctors are concerned about detection and treatment of cancers that are not life threatening. By age 70, roughly 70% of men may have detectable cancer in their prostate. Most of this cancer is indolent, with some estimates suggesting that more than 90%, probably over 95%, of these prostate cancers are slow-growing and not likely to cause death. Indolent cancers are usually defined as small (less than 0.5 cc by some researchers) and low Gleason score (6 or less or 3+4 or less). In most cases, treatment of these cancers does not prolong life and has a high chance of reducing the patient's quality of life due to the pain and risks of treatment such as surgery, plus side effects such as impotence and incontinence. Over-treatment often occurs when BPH and/or prostatitis cause an increase in PSA that triggers a biopsy that inadvertently discovers a small, slow-growing cancer. The cancer may not produce much PSA or present a serious health risk, but is typically nonetheless treated. Fear by the man and his family and pressures from the medical system often lead to treatment that would have been unnecessary if the biopsy had not been performed.

These weaknesses in currently used PSA screening methods have led the U.S. Preventative Services Task Force to recommend elimination of routine PSA testing for most men. However, new screening methods that can address these problems would greatly increase prostate cancer survival, reduce recurrence, and reduce the financial, social, and health costs of treating patients who have prostate conditions.

### SUMMARY OF THE INVENTION

The present invention provides a method to improve upon prostate cancer screening, thereby saving lives and reducing morbidities of unwarranted biopsies and over-treatment. The methods use a systematic analysis of the growth of PSA, PSA variation and/or other information and analyses to distinguish prostate conditions, including, for example, high-risk cancers from no cancer. Approaches include Dynamic-Differential Strategy, Dynamic-Differential Analysis, and Dynamic Analysis.

In one aspect, the invention encompasses a method for estimating the probability of a prostate condition in a subject, comprising: a) obtaining a series of at least a first and a second PSA value from said subject, wherein the PSA values are measured in the subject at at least a first and a second time; b) performing a dynamic analysis using a computer system, wherein said dynamic analysis comprises fitting said series of PSA values to a functional form equation to form a fitted trend over time and calculating a characteristic of said fitted trend, wherein said characteristic reflects PSA variation; and c) estimating the probability of said prostate condition by comparing said characteristic with results based on analysis of population data.

In some embodiments, performing said dynamic analysis further comprises: calculating a tolerance range of said fitted trend; removing a PSA value from said series of PSA values that has a value outside said tolerance range, thereby forming a subseries of PSA values; and fitting said subseries of PSA values to a functional form equation to form a second fitted trend over time and calculating a characteristic of said second fitted trend; wherein estimating the probability of said prostate condition further comprises comparing said characteristic of said second fitted trend with results based on analysis of population data.

In some embodiments, calculating said characteristic of said fitted trend comprises weighting the contribution of said first PSA value to said characteristic differently than the contribution of said second PSA value to said characteristic. In some embodiments, said first PSA value is measured before said second PSA value, and said contribution of said first PSA value is weighted less than said contribution of said second PSA value.

In some embodiments, calculating said characteristic of said fitted trend comprises weighting the contribution of said first PSA value to said characteristic the same as the contribution of said second PSA value to said characteristic.

In some embodiments, the method further comprises (d) selecting a target PSA value from said series of PSA values, wherein said target PSA value is measured at a target time; (e) calculating a trend PSA value based on said functional form equation for said target time; and (f) calculating a characteristic of said trend PSA value, wherein said characteristic reflects a comparison of said trend PSA value and said target PSA value, and wherein estimating the probability of said prostate condition further comprises comparing said characteristic of said trend PSA value with results based on analysis of population data. In some embodiments, the characteristic of said trend PSA value is a difference between said trend PSA value and said target PSA value. In some embodiments, the characteristic of said trend PSA value is the difference between said trend PSA value and said target PSA value, divided by said trend PSA value.

In some embodiments, the method further comprises d) obtaining a third PSA value, wherein said third PSA value is measured in the subject at a third time, wherein said third time is subsequent to said at least first and second times; e) projecting said fitted trend using said computer system to said third time to calculate a projected PSA value at said third time; and f) calculating a characteristic of said projected PSA value, wherein said characteristic reflects a comparison of said projected PSA value and said third PSA value, and wherein estimating the probability of said prostate condition further comprises comparing said characteristic of said projected PSA value with results based on analysis of population data. In some embodiments, the characteristic of said projected PSA value is a difference between said projected PSA value and said third PSA value. In some embodiments, the characteristic of said projected PSA value is the difference between said projected PSA value and said third PSA value, divided by said projected PSA value.

In another aspect, the invention provides a method for estimating the probability of a prostate condition in a subject, comprising: a) obtaining a series of at least two PSA values from said subject, wherein the PSA values are measured in the subject at at least two different times; b) performing a dynamic analysis using a computer system, wherein said dynamic analysis comprises fitting said series of PSA values to a functional form equation to form a fitted trend over time; c) selecting a target PSA value from said series of at least two PSA values, wherein said target PSA value was measured at a target time; d) calculating a trend PSA value based on said functional form equation for said target time; e) calculating a characteristic of said trend PSA value, wherein said characteristic reflects a comparison of said trend PSA value and said target PSA value; and f) estimating the probability of said prostate condition by comparing said characteristic of said trend PSA value with results based on analysis of population data. In some embodiments, the characteristic of said trend PSA value is a difference between said trend PSA value and said target PSA value. In some embodiments, the characteristic of said trend PSA value is the difference between said trend PSA value and said target PSA value, divided by said trend PSA value.

In another aspect, the invention provides a method for estimating the probability of a prostate condition in a subject, comprising: a) obtaining a series of at least a first and a second PSA value from said subject, wherein the PSA values are measured in the subject at at least a first and a second time; b) performing a dynamic analysis using a computer system, wherein said dynamic analysis comprises fitting said series of PSA values to a functional form equation to form a fitted trend over time; c) obtaining a third PSA value, wherein said third PSA value is measured in the subject at a third time, wherein said new time is subsequent to said at least first and second times; d) projecting said fitted trend using said computer system to said third time to calculate a projected PSA value at said third time; e) calculating a characteristic of said projected PSA value, wherein said characteristic reflects a comparison of said projected PSA value and said third PSA value; and f) estimating the probability of said prostate condition by comparing said characteristic of said projected PSA value with results based on analysis of population data.

In some embodiments, performing said dynamic analysis further comprises: calculating a tolerance range of said fitted trend; removing a PSA value from said series of PSA values that has a value outside said tolerance range, thereby forming a subseries of PSA values; and fitting said subseries of PSA values to a functional form equation to form a second fitted trend over time and calculating a characteristic of said second fitted trend; wherein estimating the probability of said prostate condition further comprises comparing said characteristic of said second fitted trend with results based on analysis of population data.

In some embodiments the characteristic of said projected PSA value is a difference between said projected PSA value and said new PSA value. In some embodiments, the characteristic of said projected PSA value is the difference between said projected PSA value and said new PSA value, divided by said projected PSA value.

In some embodiments of the invention, said prostate condition is selected from the group consisting of: prostatitis, benign prostate hyperplasia, prostate cancer, and no prostate disease. In some embodiments, said subject is a human. In some embodiments, said computer system comprises a device for network communication, a storage unit, and a processor. In some embodiments, the functional form equation takes the form of PSA(t)=PSAn+M*ê(PSAgr*t), wherein t is the time, PSAn is a constant reflecting baseline PSA, M is a constant multiplier, and PSAgr is a constant reflecting the exponential growth rate of PSA due to cancer.

In another aspect, the invention provides a computer implemented method for analyzing the results of at least two PSA tests for a subject, comprising: a) obtaining a series of at least two PSA values from said subject, wherein the PSA values are measured in the subject at at least two different times; and b) performing a dynamic analysis using a computer system; wherein said dynamic analysis comprises fitting said series of PSA values to a functional form equation to form a fitted trend over time; wherein the functional form equation takes the form of PSA(t)=PSAn+M*ê(PSAgr*t), and wherein t is the time, PSAn is a constant reflecting baseline PSA, M is a constant multiplier, and PSAgr is a constant reflecting the exponential growth rate of PSA due to cancer; and c) outputting the fitted trend on by an output device.

In some embodiments, said computer system comprises a computer program product stored on a non-transient computer medium, wherein said computer program product comprises computer-readable instructions for performing said dynamic analysis. In some embodiments, obtaining said series of PSA values comprises obtaining at least three PSA values from said subject, wherein the PSA values are measured in the subject at at least three different times. In some embodiments, PSAn is calculated based on analysis of population data.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows receiver operating characteristic (ROC) curves for various settings. The area under the curve (AUC) values are: D-D Strategy 0.95; Dynamic-Differential 0.88; Dynamic Analysis 0.84; Static PSA 0.75. The AUC for a coin toss is 0.50.

FIG. 2 contains graphs depicting example data sets and trends for calculating TJump or TDrop for Dynamic Analysis.

FIG. 3 contains graphs depicting example death risks for smooth PSA trends.

FIG. 4 depicts example death risk thresholds for smooth PSA trends.

FIG. 5 contains graphs depicting example death risks for variable PSA trends.

FIG. 6 depicts example death risk thresholds for variable PSA trends.

FIG. 7 shows examples of Dynamic Differential Analysis methods based on PJump/PDrop and New Trend methods.

FIG. 8 shows an example of Dynamic Differential Strategy.

FIG. 9 depicts an example of a strategy process for a subject at age 52.

FIG. 10 depicts an example of the strategy process for a subject at age 54.

FIG. 11 depicts an example strategy process for a subject at age 56.

FIG. 12 depicts an example strategy process for a subject at age 57/58.

FIG. 13 is a graph showing example weighted risk of recurrence and unwarranted biopsy for static PSA screening.

FIG. 14 is a graph showing example weighted risk of recurrence and unwarranted biopsy for Dynamic Differential Strategy.

FIG. 15 is a graph showing example weighted risk of recurrence, biopsy risk, and treatment risk.

FIG. 16 shows some non-limiting examples of key variables that affect risk and their risk hierarchy.

FIG. 17 depicts an example set of curves that plot Biopsy %, Biopsy Cost vs Recurrence, and Biopsy Risk % vs PSA.

FIG. 18 depicts an example set of curves that plot Treatment %, Recurrence %, and Recurrence Risk % vs PSA.

FIG. 19 depicts an example set of curves that plot Treatment %, Treatment Cost vs Recur, and Treatment Risk % vs PSA.

FIG. 20 depicts an example set of curves that plot Biopsy %, Cancer %, and Treatment % vs PSA.

FIG. 21 shows a projected scenario for a subject using static PSA screening starting from a PSA of 3.0.

FIG. 22 shows a projected scenario for a subject using static PSA screening starting from a PSA of 5.0.

FIG. 23 shows a projected scenario for a subject using static PSA screening starting from a PSA of 7.0.

FIG. 24 depicts an example projection based on Dynamic Differential Strategy for a subject with a PSA of 3.0.

FIG. 25 depicts example ROC curves for Dynamic Analysis and PSA Screening.

FIG. 26 depicts percentage of high PSAgr cancers mixed for Dynamic Analysis and Static PSA screening.

FIG. 27 shows six calibrated and adjusted PSA test results for an example subject over five years.

FIG. 28 shows example estimated and projected risks of unwarranted biopsy, recurrence if cancer, and overall recurrence for Dynamic Analysis.

FIG. 29 shows risk curves for unwarranted biopsy and for detecting cancer in an example case with high PSAgr and low PSAvar.

FIG. 30 shows a relationship between the chance of detecting cancer, the risk of recurrence if cancer, and the overall risk of cancer.

FIG. 31 shows risks of unwarranted biopsy, recurrence if cancer is present, and overall vs PSA for an example case with high PSAgr and low PSAvar.

FIG. 32 depicts example variation in PSA data.

### DETAILED DESCRIPTION OF THE INVENTION

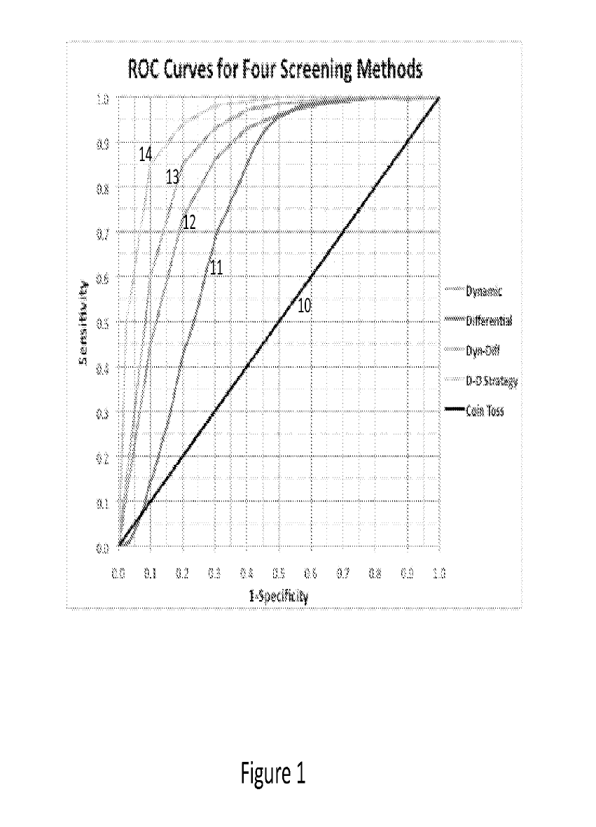

The present invention provides methods, systems, and software to improve screening for prostate conditions, thereby saving lives and reducing morbidities. Prostate conditions include but are not limited to healthy prostates; prostate cancer, such as fast-growing or slow-growing cancers; benign prostate hyperplasia (BPH); and prostatitis, such as prostatitis due to inflammation or due to infection. The methods use a systematic analysis of the growth of PSA, PSA variation and/or other information and analyses to distinguish fast-growing, high-risk cancers from no cancer or low-risk cancer. Approaches include Dynamic-Differential Strategy, Dynamic-Differential Analysis, Dynamic Analysis, and Differential Analysis. Methods suitable for use with these approaches are disclosed in U.S. Publication Nos. 2008/0033253, 2009/0062624, 2009/0088981, 2010/0049546, and 2010/0168621, each herein incorporated by reference in its entirety. FIG. 1 depicts the ROC curves for each of these methods compared to a random “coin toss” guess (10), showing that the four methods have high sensitivity with low false positive rates. Differential Screening (11) has a higher area under the ROC curve (AUC) than a coin toss. Dynamic Screening (12) has a higher AUC than Differential Screening. Dynamic Differential Screening (13) has a higher AUC than Dynamic Screening. Dynamic Differential Strategy screening (14) has the highestAUC.

Screening for prostate cancer is controversial, especially when only a single PSA test is compared to a threshold, such as 4.0 ng/mL. Comparisons of a single PSA test to such static prostate biomarker thresholds are referred to as “static” screening. Several recent studies have raised concerns about the value of static PSA screening to detect prostate cancer. A study in the US found no decrease in prostate cancer mortality associated with PSA screening. (See Andriole et al., New England Journal of Medicine, 2009.) Another study in Europe did find that static PSA screening reduced mortality by 20% but at the average cost of screening 1,410 men and unnecessarily treating 48 men to prevent one prostate cancer death. (See Schroder et al., New England Journal of Medicine, 2009.)

The American Cancer Society and the American Urological Association have recently revised their screening recommendations to reflect this controversy over static PSA testing and PSA testing in general. The U.S. Preventative Services Task Force now recommends elimination of routine PSA testing for most men. Valuable new methods are ones that work well at low PSAs for early detection and also achieve low risk of unwarranted biopsies that do not find cancer or that lead to over-treatment of indolent cancer. Screening methods that reduce unwarranted biopsies will also reduce over-treatment.

### Screening and Evaluation Methods

In some embodiments, the methods of the invention include at least one of: Dynamic Analysis, Dynamic Differential Analysis and Dynamic Differential Strategy. Each method is introduced in the sections below. For Dynamic Differential Analysis and Dynamic Differential Strategy, a Monte Carlo simulator was developed that is calibrated to published data. The Monte Carlo system can simulate a large synthetic population of men that reflects older U.S. men in terms of prostate volume growth from BPH, prostatitis and significant progressing cancer. One challenge is proper evaluation of men with both progressing cancer and prostatitis and/or BPH. However, progressing cancer is statistically independent of BPH and prostatitis, or very nearly independent. Therefore, the Monte Carlo system can reliably combine BPH and/or prostatitis and progressing cancer, and other important aspects. Prospective studies can confirm performance realized on simulated populations.

In some embodiments, the simulation system builds up a population of men one man at a time. In some embodiments, the simulation system considers each man when he reaches a threshold age for prostate conditions, for example at age 40. In some embodiments, multiple ages are considered. The man may have a normal prostate or have one or more prostate conditions over time, such as prostate enlargement from BPH, prostatitis caused by infection and/or inflammation and prostate cancer of varying severity and starting points. In some embodiments, for each man and at each age considered, the Monte Carlo system draws, from available population distributions, a time-dependent pattern of incidence and severity of benign prostate conditions, such as prostate volume. In some embodiments, the available population distributions are based on data from medical studies, which may be published or unpublished.

In some embodiments, the simulation system then draws corresponding outcome distributions for the pattern of incidence of severity simulated. In some embodiments, the outcomes are based on available data, including but not limited to medical journal articles, data from private or public institutions, and/or unpublished data or analyses. Possible outcomes that are drawn include but are not limited to levels of PSA and Free PSA over time, as well as other biomarkers.

For prostate cancer, there is a great deal of knowledge of its incidence, severity and outcomes, including corresponding levels of PSA and Free PSA, which can be used by the simulation system. The longest time patterns have been captured by the Baltimore Longitudinal Study of Aging using frozen serum for PSA biomarker tests prior to the PSA era—with many articles published on these data. Medical evidence suggests that prostate cancer progression is independent of benign conditions, or is at least poorly correlated with benign conditions. This practical independence allows the Monte Carlo system, in some embodiments, to estimate PSA and Free PSA values for the same man from independent draws from probability distributions for benign conditions and for progressing prostate cancer.

In some embodiments, the distributions for each prostate condition (incidence, severity and outcomes) are tuned so that the overall synthetic population conforms to measured population distributions. In some embodiments, distributions in the simulation model are tuned to produce a synthetic population of men without prostate cancer that is consistent with the distributions for every age, for some ages, or for a single age. One example distribution suitable for use in tuning is provided by a Center for Disease Control (CDC) study, which studied the distribution of PSA and Free PSA by age in men without evidence of prostate cancer. Another example distribution is provided by Mayo Clinic researchers, who studied the distribution of prostate volume and PSA by age for a representative population. In some embodiments, results obtained from the methods described herein contribute to a data set used for simulation or distribution tuning In some embodiments, a reasonably representative synthetic population of men and prostate conditions is considered to be achieved when all the condition-specific distributions conform to results found in the literature and the overall outcome distributions conform to population distributions found in the literature. Such a synthetic population is suitable for use in testing the methods described herein, and/or for comparison with static PSA screening. Other populations, both synthetic and non-synthetic, are also suitable for use in testing the methods described herein and/or for comparison with static PSA screening.

### Prostate Biomarkers and Secondary Values

In some embodiments of the invention, PSA values are used for the screening methods described herein. In some embodiments, other prostate biomarkers are used, which include but are not limited to FPSA, ProPSA, and PCA3, a genetic urine marker.

Free PSA typically refers to the subset of total PSA in blood that is not attached to protein. It is measured using blood testing methods similar to those used for total PSA. In some embodiments, Free PSA (FPSA) or secondary values derived from FPSA are used for the screening methods described herein. In some embodiments, FPSA is used in combination with another biomarker, such as with PSA. In a non-limiting example, the ratio of Free PSA to PSA (FPSA %) is used. FPSA % can increase sensitivity and specificity compared to PSA alone or FPSA alone. In some embodiments, FPSA refers to or includes ProPSA, one form of Free PSA, which has been shown to increase sensitivity and specificity even more than using general FPSA. In some embodiments, a combination of PSA, FPSA, ProPSA and/or PCA3 are used rather than PSA only.

In some embodiments, prostate volume is used for screening. Cancers in very small prostates have an 8.4 times greater relative risk of time to progression than cancers in very large prostates for men in the VA SEARCH database. PSA for very small prostates is less than 20% of the PSA from very large prostates. Therefore, in some embodiments, PSA thresholds are much lower for very small prostates (greater danger with less chance of false positives) and much higher for very large prostates. In some embodiments, ultrasound volume measurements are used as part of the differential process of prostate cancer screening. In such embodiments, detection of an enlarged prostate for early treatment is an additional advantage.

In some embodiments, secondary values derived from one or more biomarker values are used, including but not limited to velocity (e.g. PSAV, FPSAV, ProPSAV), growth rate (PSAgr, FPSAgr, ProPSAgr), and variability (PSAvar, FPSAvar, ProPSAvar). Non-limiting examples of how to calculate such secondary values for PSA are provided below. One skilled in the art would understand how to apply the methods of calculating the secondary values to other prostate biomarkers.

In some embodiments, PSA velocity (PSAV), FPSAV, and/or ProPSAV is used for prostate condition screening. PSA Velocity (PSAV) has added value to the use of PSA as a marker for cancer. (Carter et al., J Natl Cancer Inst, 2006; D'Amico et al., JAMA, 2005) Recent studies have shown that PSAV better distinguishes between the most dangerous cancers and indolent cancer. In some embodiments, PSAV is the annual rate of change in PSA. In some embodiments, FPSAV is the annual rate of change in FPSA. In some embodiments, such as in Dynamic Analysis methods as described herein, velocities are measured as the slope (first time derivative) of the PSA and/or FPSA trend functions at any point in time. Typically, velocity at the time of the most recent PSA test is used for screening and analysis, including but not limited to analysis by comparing velocity to a threshold.

In 2006, Carter at Hopkins and his NIA colleagues published an article on the use of PSAV to distinguish early between prostate cancer that lead to death on the one hand and, on the other, no cancer and cancers that did not lead to death (Carter et al., 2006). Their study analyzed data for 980 men who were participants in the Baltimore Longitudinal Study of Aging (BLSA) for up to 39 years. Carter found that PSA velocity may help to identify men with life-threatening prostate cancer during a period when their PSA levels are associated with the presence of curable disease. Another study showed that PSAV greater than 0.4 ng/ml per year was positively associated with high-Gleason grade disease, positive surgical margins and seminal vesicle invasion at RP. PSAV screening was also associated with a 50% reduction in insignificant disease. (Loeb et al., J Urol., 2010) Another study showed that the preoperative PSAV was a significant independent predictor of the Gleason score and non-organ confined disease in the RP specimen. (Loeb et al., Urology, 2008) In these studies, PSAV was estimated using a linear function and a few PSA test values.

In some embodiments, the growth rate of PSA (PSAgr) is used for prostate condition screening. In some embodiments, PSAgr is calculated as the ratio of PSAV/PSA. Studies suggest that both PSAV and PSA are significantly useful in multivariate analyses.

The BLSA study described above also shows mixed effects estimates of high and low PSA growth rates (95% confidence ranges) for men who died of prostate cancer, men who did not die from their cancer and men who did not have prostate cancer. Mixed effects typically refer to effects that are due in part to both fixed and random or variable effects. Reanalysis of the published results using the methods of the invention found similar patterns in PSA growth rates (PSAgr): 20% PSAgr for men who died of prostate cancer, 12% PSAgr for men with no cancer and 11% PSAgr for men with cancer who did not die from prostate cancer. This evidence suggests that PSA growth rate may be a useful screening tool and an indicator of the most deadly cancers.

In some embodiments, PSAgr is a variable that is derived from a best fit equation, such as a functional form equation for dynamic analysis as described herein.

In some embodiments, PSA variability is used for prostate condition screening. PSA variability can refer to variation of one or more PSA values from a trend, including but not limited to trends generated by Dynamic Analysis. In some embodiments, PSA variability refers to the variation of a single PSA value from a trend, such as a Jump or Drop or a projected Jump or Drop. In some embodiments, PSA variability refers to PSA variation (PSAvar), which reflects variation of a PSA data set from the trend generated from that data set. Further analysis according to the methods of the invention described herein found that PSA variability can help distinguish between increasing PSA caused by progressing cancer and by other causes. Without being bound by theory, it is suggested that PSA from cancer tends to grow exponentially more smoothly than PSA from prostatitis. The back and forth battle between infection and/or inflammation and a body's defenses may cause PSA to bounce up and down and often causes variability around an increasing trend. As used herein, smoothness refers to low variability. In some embodiments, smooth PSA growth refers to PSA values that increase with low variation with respect to a fitted trend. In some embodiments, smooth PSA growth refers to few or no significant Jumps or Drops in the data set with respect to a fitted trend.

In some embodiments, the relationship between two biomarkers, such as FPSA and PSA, is used for prostate cancer screening. In some embodiments, the relationship is measured using a ratio. Such ratios include but are not limited to FPSA %, FPSAV %, and FPSAΔ %. FPSA % refers to the ratio of FPSA to PSA. FPSAV % refers to the ratio of FPSAV to PSAV. FPSAΔ % refers to the ratio of FPSAΔ to PSAΔ. In some embodiments, PSAΔ is the change in PSA after differential treatment with antibiotics and/or anti-inflammatory medications. In some embodiments, FPSAΔ is the change in FPSA after differential treatment. An example of how to use FPSA and PSA ratios to distinguish between different prostate conditions is provided in Example 2. Other ratios include but are not limited to ProPSA % (ProPSA/PSA), ProPSAV % (ProPSAV/PSAV), and ProPSAΔ % (ProPSAΔ/PSAΔ).

### Static Screening—Statistical Performance

FPSA % has substantially improved sensitivity and specificity compared to static PSA screening for many studies over many years. Screening performance is sometimes measured by the combination of sensitivity and specificity. In some embodiments, receiver operating characteristic (ROC) curves are used to summarize the combined results. In some embodiments, the area under the ROC curve (AUC) is used as a single summary measure. The FDA has approved the use of FPSA % as a way to reduce unwarranted biopsies. A recent study of ProPSA, FPSA and PSA found that FPSA % alone increased the AUC 21% points to 77% compared to 56% for static PSA alone. ProPSA % alone increased the AUC by only 1% AUC point over FPSA % alone to 78%.

### Trend Functions

Various equations can be used to fit a trend to the prostate biomarker data set. Such equations can be referred to as trend functions or functional form equations. In some embodiments, the trend function is linear, such as the equation PSA(t)=PSAn+PSAV*t, where PSAn is an initial PSA and PSAV reflects the annual change in PSA. In some embodiments, the trend function is a polynomial, including but not limited to a polynomial of degree 2. In some embodiments, the trend function is logarithmic or exponential. In some embodiments, different subsets of a PSA data set can be fitted to different trend functions.

In one non-limiting example, the trend function is an exponential plus constant function, taking the form of PSA(t)=PSAn+M*ê(PSAgr*t). PSAn reflects the contribution of baseline PSA, e.g. PSA that is present in the subject in the absence of any cancer. In some embodiments, PSAn is a constant. In some embodiments, PSAn is calculated from the fit equation. In some embodiments, PSAn is set based on analysis of population data. For example, in some studies, the average PSAn is about 1.0. In some embodiments, PSAn is about 0.7, about 0.8, about 0.9, about 1.0, about 1.1, about 1.2, about 1.3, about 1.4, or about 1.5. In some embodiments, PSAn can vary with time. In one non-limiting example, PSAn can be a linearly increasing function. PSAgr reflects a growth rate of the cancer, which in some embodiments reflects the aggressiveness of the cancer. M is a multiplier, typically a constant multiplier. In some embodiments, PSAc is calculated as M*ê(PSAgr*t), which in some embodiments reflects cancer progression (such as tumor size or spread). In some embodiments, PSAV is calculated as the derivative of PSA(t). For the exponential plus constant function described above, PSAV=PSAgr*PSAc.

In some embodiments, the functional form takes the form PSA(t)=A*t̂2+B*t+C.

In some embodiments, the functional form takes the form PSA(t)=PSAn+M*X̂(PSAgr*t), where X can be any constant. In some embodiments, the functional form takes the form PSA(t)=A+B*Log(C*t+D), Log(PSA(t))=A+B*t, Log(PSA(t))=A+B*t+C*t̂2, or Log(PSA(t))=A+B*X̂(C*t), where Log can be the natural log.

Trend functions of the invention can be used to fit data sets comprising values of any prostate biomarker as described herein. In some embodiments, trends are fit to other biomarkers, including but not limited to FPSA(t) and ProPSA(t) and their ratios, such as FPSA % (t) and ProPSA % (t), where a ratio is calculated at each test time and a trend is fit through the resulting ratios. In some embodiments, the ratio of fitted trends can be calculated. One non-limiting example is TrendFPSA % (t)=FPSA(t)/PSA(t). In some embodiments, trend functions are fit based on at least 2, at least 3, at least 4, at least 5, at least 6, at least 7, at least 8, at least 9, at least 10, at least 11, at least 12, at least 15, or at least 20 values of a biomarker. In some embodiments, each value is measured at a different time. For example, each value can be measured at least a day, at least a week, at least a month, at least 3 months, at least 6 months, at least 8 months, or at least a year from a previously measured biomarker value.

### Dynamic Analysis

In some embodiments, Dynamic Analysis according to the invention uses Bayesian pattern recognition methods to help distinguish between high-risk cancer and no cancer, between high-risk cancer and lower-risk cancer, between large cancer and no cancer, or between other combinations of prostate conditions.

Signal processing variables include:
Cancer PSA growth rate (PSAgr)
PSA variation (PSAvar)
PSA acceleration (PSAacc)

PSA acceleration (PSAacc) is a measure of the extent trend PSAV is increasing or decreasing. In some embodiments, PSAacc can be calculated as the rate of increase per year of PSAV at a point in time (PSAA) divided by the PSAV at that point in time (PSAacc=PSAA/PSAV). PSAV is the slope of the PSA trend at a point in time (first derivative of PSA trend). PSAA is the slope of the PSAV trend (second derivative of PSA trend).

PSAacc can be valuable because PSA from progressing cancer rarely decelerates. In contrast, PSA from prostatitis eventually decelerates, especially if treated with antibiotics and anti-inflammatory meds.

In some embodiments, PSAacc is calculated as a second derivative of a functional form trend equation. In some embodiments, a 2nd-order polynomial trend is fit to a man's full PSA test history. The second derivative of this trend captures the acceleration of the PSA level. That is to say, if the functional form of the 2nd-order trend is f(x)=ax̂2+bx+c, then the second derivative is f″(x)=2a. In some embodiments, this value is then converted into a quantity that describes the proportion by which the PSA velocity is changing on an annual basis, by dividing the ‘2a’ value by the PSAV at a time point two years back from the most recent date. If this PSAV value is a very small positive quantity or any negative quantity, the final acceleration metric is given a special value, set aside, and considered to be not of interest to our investigation, which is only concerned with men with positive velocities, specifically distinguishing those men with cancer from those without.

In some embodiments, Dynamic Analysis considers other factors that can affect PSA, cancer risk, treatment outcomes, and/or other screening risks, including but not limited to age, race, weight, life expectancy, body mass index (BMI), blood pressure, drug use, other medical conditions, and results of medical tests. Age affects the prior probabilities of elevated PSA and the threat of progressing prostate cancer. Race seems to matter. BMI seems to affect the risk of bad cancers with obese men at higher risk, perhaps from hemo-dilution of PSA values. Use of statins seems to reduce the risk of fatal prostate cancer. Digital rectal exams seem to provide information, although they are highly subjective and may lead to false positives.

### Dynamic Analysis—PSAgr, PSAvar

Dynamic analysis of PSA growth rate and variation can distinguish between high Gleason cancers and no cancer. In some embodiments, risk assessments are performed using dynamic analysis based on estimates of cancer PSA growth rates (PSAgr) and PSA variation (PSAvar) to distinguish between high Gleason cancers and no cancer and help identify the most deadly cancers.

In a study presented in Example 1, 670 men from the Tyrol screening project had no cancer detected by biopsy and at least 5 PSA tests over 4 years with no gap more than 2 years. 370 men from the Tyrol underwent surgery (RP) and had pathological results and the same minimum PSA history. Men with Gleason scores of 4+3 or greater and stage T2b and greater were considered high risk. Risk assessment was performed using receiver operating characteristic curves (ROC) and the percentage of high PSAgr (>15%) cancers missed at a given sensitivity.

In some embodiments, dynamic analysis according to the invention produces ROC curves with higher AUC than ROC curves using a single static PSA (e.g., 0.86 vs 0.79 for full PSA history). At any given specificity, Dynamic Analysis misses fewer high PSAgr cancers than static analysis. Thus, dynamic analysis using PSAgr and PSAvar can help improve sensitivity to the most serious cancers and specificity to no cancer found by biopsy. In addition, Dynamic Analysis misses a lower proportion of high PSAgr cancers, which may pose higher risks of death.

### Faster Exponential Growth Predicts the Most Deadly Cancers

Estimates of PSAgr can also be used to distinguish between deadly and less deadly cancers as evidenced by survival statistics. We conducted a study of BLSA men with cancer detected prior to the PSA era, presumably by symptoms, and the subset of those men who died of prostate cancer. We used Dynamic Analysis methods to divide the men into fast and slow growing cancer PSA groups (high and low PSAgr) and used Kaplan-Meier methods to estimate cancer specific death vs years after cancer PSA (PSAc) reached 3.0, which is a 4.0 overall threshold for a typical man with a median no cancer PSA of 1.0. We found that fast growing cancer (high PSAgr) kills much faster than slow growing cancer (low PSAgr).

15 years after reaching a 3.0 PSAc:
60% of high PSAgr men died.
0% of low PSAgr men died.

20 years after reaching a 3.0 PSAc:
80% of high PSAgr men died.
22% of low PSAgr men died.

### PSA Variability

In some embodiments, PSA variability is measured by PSA variation (PSAvar). PSA variation is a measure of the dispersion of a man's PSA tests about the fitted trend. In some embodiments, the measure considers all or most of the PSA tests. In some embodiments, variation is calculated as the average difference of each PSA test from the fitted trend. In some embodiments, variation is calculated as the average percent difference of each PSA test from the fitted trend. In some embodiments, variation is calculated as a standard deviation, a standard deviation of the percent difference, a standard error, a range, a mean difference, a median deviation, a variance, a coefficient of variation, or a relative mean difference. Other methods of calculating variation are known in the art and suitable for calculating PSAvar.

In some embodiments, for each PSA test value a deviation or difference from the fitted trend at that time of the test is calculated: Diff(t)=TestPSA(t)−TrendPSA(t). For each PSA test value, a percentage deviation or percentage difference from the fitted trend at that time of the test can be calculated: % Diff(t)=Diff(t)/TrendPSA(t). In some embodiments, weights can be used to emphasize some deviations or differences more than others, where the weight for each test at its time is W(t). In embodiments with no weighting, W(t)=1. In some embodiments, weights are chosen that sum to the number of tests, where: ΣW(t)=n, where n equals the number of tests.

Four example measures of PSA variation around the trend are: average absolute differences from the trend (AAD), average absolute percentage differences from the trend (AAPD), standard deviation of differences from the trend (SDD) and standard deviation of percentage differences from the trend (SDPD). Formulas for these example measures are shown below where the sum of the weights equals the number of tests:

AAD=(ΣW(t)*ABS[Diff(t)])/n

AAPD=(ΣW(t)*ABS[% Diff(t)])/n

SDD=([ΣW(t)*Diff(t)̂2]/[n−1])̂0.5

SDPD=([ΣW(t)*% Diff(t)̂2]/[n−1])̂0.5

Additional example formulae include:

$$A A D = \frac{{\sum }_{t = 0}^{n - 1} {w}_{t} \vert {Diff}_{t} \vert}{{\sum }_{t = 0}^{n - 1} {w}_{t}} = \frac{{\sum }_{t = 0}^{n - 1} {w}_{t} \vert {Diff}_{t} \vert}{n}$$

$$A A P D = \frac{{\sum }_{t = 0}^{n - 1} {w}_{t} \vert \% {Diff}_{t} \vert}{{\sum }_{t = 0}^{n - 1} {w}_{t}} = \frac{{\sum }_{t = 0}^{n - 1} {w}_{t} \vert \% {Diff}_{t} \vert}{n}$$

$$S D D = \sqrt{\frac{{\sum }_{t = 0}^{n - 1} {w}_{t} {Diff}_{t}^{2}}{\frac{( n - 1 ) {\sum }_{t = 0}^{n - 1} {w}_{t}}{n}}} = \sqrt{\frac{{\sum }_{t = 0}^{n - 1} {w}_{t} {Diff}_{t}^{2}}{n - 1}}$$

$$S D P D = \sqrt{\frac{{\sum }_{t = 0}^{n - 1} {w}_{t} \% {Diff}_{t}^{2}}{\frac{( n - 1 ) {\sum }_{t = 0}^{n - 1} {w}_{t}}{n}}} = \sqrt{\frac{{\sum }_{t = 0}^{n - 1} {w}_{t} \% {Diff}_{t}^{2}}{n - 1}}$$

In some embodiments, the calculation of PSA variation is time-discounted so that more recent PSA points affect the final PSAvar quantity more than points in the past. Example calculations of equally weighted and differently weighted are provided in Example 4.

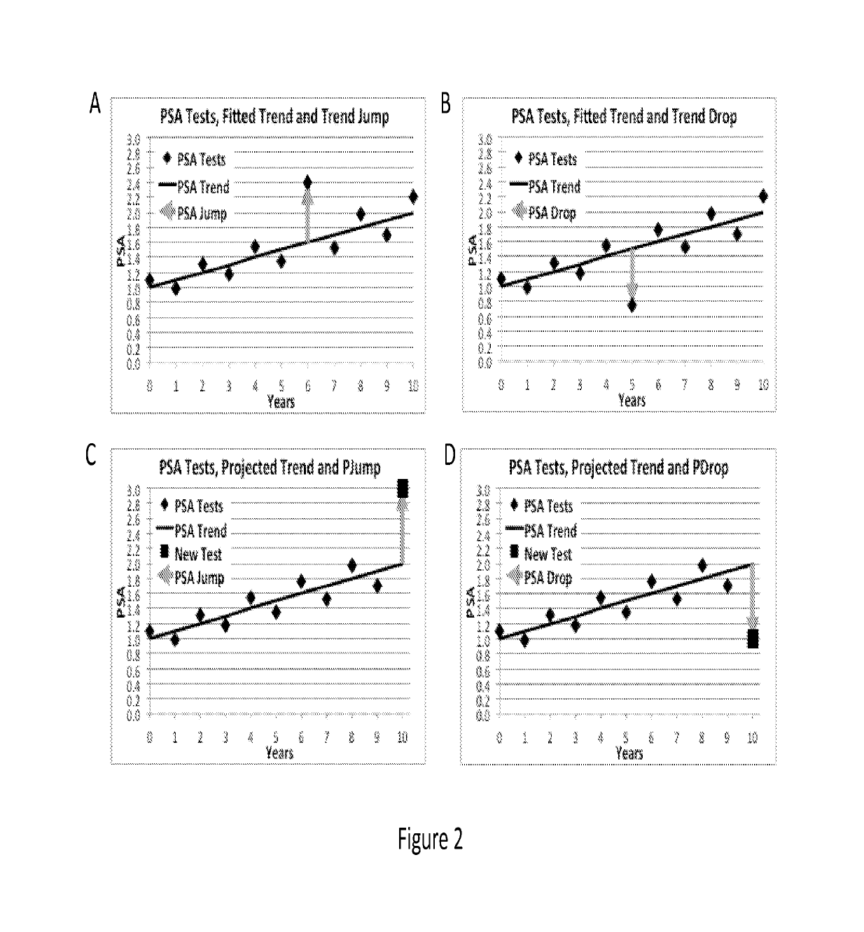

In some embodiments, PSA variability is measured as a Jump or a Drop of a PSA value compared to a fitted trend. In some embodiments, TJump is calculated if the PSA value is greater than the trend PSA value at the corresponding time, wherein: TJump is equal to the PSA value minus the corresponding trend PSA value and TJump % is equal to TJump divided by the corresponding trend PSA value. For example, at year 6 on FIG. 2A the gray arrow shows a Jump in PSA where TJump=0.8 and TJump %=50%.

In some embodiments, TDrop is calculated if the PSA value is less than the trend PSA value at the corresponding time, wherein: TDrop is equal to the corresponding trend PSA value minus the PSA value. TDrop % is equal to TDrop divided by the corresponding trend PSA value. For example, at year 5 on FIG. 2B the gray arrow shows a Drop in PSA where TDrop=0.75 and TDrop %=50%.

In some embodiments, the difference between a PSA value and the trend is calculated as the PSA value minus the corresponding trend PSA value, where a positive difference is considered a Jump and a negative difference is considered a Drop.

In some embodiments, a Jump or Drop is calculated from a projected trend. In some embodiments, an additional PSA value is measured after the PSA values used to calculate the fitted trend. The fitted trend is then projected to at least the time of the additional PSA value. For the additional PSA test value, the system calculates a Jump or Drop from the corresponding projected trend PSA value and/or the equivalent percentage: PJump (PJump %) or PDrop (PDrop %).

In one non-limiting example, a series of PSA test values are obtained from year 0 through year 10. The system removes the last PSA value from the series and then fits the remaining series of PSA values (years 0 through 9) to form a trend. It then projects the fitted trend to a projected PSA value at the time of the last PSA value, which is at year 10. PJump is calculated if the last PSA value is greater than the trend PSA value at the corresponding time, wherein: PJump is equal to the PSA value minus the corresponding projected trend PSA value and PJump % is equal to PJump divided by the corresponding projected trend PSA value. For example, at year 10 in FIG. 2C, the gray arrow shows a Jump in PSA to the black square, where PJump=1.0 and PJump %=50%.

PDrop is calculated if the last PSA value is less than the trend PSA value at the corresponding time, wherein: PDrop is equal to the corresponding projected trend PSA value minus the PSA value and PDrop % is equal to PDrop divided by the corresponding projected trend PSA value. For example, at year 10 on FIG. 2D, the gray arrow shows a Drop in PSA to the black square, where PDrop=1.0 and PDrop %=50%.

In some embodiments, a trend Jump, trend Drop, projected Jump, or projected Drop is used as a measure of the smoothness of the PSA trend. In some embodiments, the absolute value of the Jump/Drop is used as a measure of the smoothness of the PSA trend. In some embodiments, a threshold of variability is used to determine whether a Jump/Drop is significant. In some embodiments, such a threshold of variability is based on PSAvar. In some embodiments, the threshold of variability in a Jump is treated differently from variability in a Drop. In a non-limiting example, a Jump % of about 20% can be considered insignificant while a Drop % of about 20% can be considered significant for the purposes of determining smoothness of a PSA trend.

### Data Exclusion

In some embodiments, Dynamic Analysis excludes one or more biomarker data points from analysis and/or subsequent diagnosis. Example methods for data exclusion are described in U.S. Patent Publication 2009/0088981, herein incorporated by reference in its entirety.

In some embodiments, the methods described herein include a step that calculates a tolerance and manages the data used for analysis. In some embodiments, the method comprises calculating a first fitted trend based on a first data set. In some embodiments, the first data set comprises all available data for one or more biomarkers. In some embodiments, the first data set comprises a subset of all available data for one or more biomarkers. In some embodiments, a tolerance region or tolerance range is calculated based on the first fitted trend. In some embodiments, the tolerance region or range is calculated based on variability or variation in the biomarker(s). In some embodiments, at least one data point that lies outside the tolerance range is excluded, generating a second, reduced data set. In some embodiments, ratios of data points or biomarker values are used to determine whether a data point lies outside a tolerance range. Typically, the second data set is used to calculate a second fitted trend. It is expected that the second fitted trend has a lower variability than the first fitted trend. The steps of calculating a tolerance range or region, excluding data, and calculating another fitted trend can be repeated as necessary.

In some embodiments, the method includes an inner decision loop that excludes test results until all included test results are within the tolerance region of the trend. In some embodiments, pairs of tests farthest from tolerance are removed during each iteration until all remaining test results are within the tolerance region. In some embodiments, a set number or fraction of tests are removed. In some embodiments, multiple trends are calculated for different purposes.

In some embodiments, the last calculated fitted trend is used for subsequent analysis steps. In some embodiments, subsequent analysis steps incorporate information from more than one of the trends calculated during data exclusion. In a non-limiting example, PSAgr from the last fitted trend and PSAvar from the first fitted trend are used for subsequent analysis. In this example, PSAgr from the last fitted trend is considered to be more accurate due to data exclusion, while PSAvar for the first trend more accurately reflects variability in PSA of the entire set of tests.

Calculating Death Risk from Prostate Cancer

The risk of prostate cancer specific death a certain number of years in the future after primary treatment (often surgery or radiation) is the best indicator of the worst cancers and the effectiveness of treatment. In some embodiments, Dynamic Screening variables are used to predict the risk of cancer specific death in the future after treatment. Dynamic Screening variables include but are not limited to: Cancer PSA (PSAc); growth rate in cancer PSA (PSAgr); PSA variability around trend (e.g. PSAvar or jumps/drops).

In some embodiments, the methods of the invention include treatment at the time the variables are at certain levels. In some embodiments, prostate cancer is confirmed by biopsy prior to treatment.

### Years to Death Smooth Trend

Death risk increases as PSAc at the time of treatment increases (e.g., as cancer progresses). The longer treatment (and biopsy) is delayed, the more PSAc increases and the greater the chance of recurrence and ultimately death from prostate cancer. Death risk is higher for higher growth rates in cancer PSA (e.g., higher PSAgr), because those cancers are more likely to be more aggressive (and thus tend to be harder to treat and more deadly).

In some embodiments, death risk is assumed to be higher for smooth trends than for variable trends with higher PSA variability around the trend. Without being limited by any theory, smooth PSA trends are typically less likely to be affected by prostatitis or other non-cancer conditions that affect PSA, and therefore smooth trends are more likely to reflect progressing cancer. In contrast, PSA that varies substantially around the trend is more likely to be caused by prostatitis or other non-cancer conditions, and the cancer, if detected, is more likely to be indolent, with low Gleason scores. In such embodiments, death risk is assumed to be lower for variable trends.

In some embodiments, the death risk is calculated based on the risk of recurrence after treatment. In some embodiments, the death risk is a function that depends on at least one of PSAV, PSAgr, PSAc, PSAvar, PSA variability, and expected lifespan. In some embodiments, at least one of PSAgr, PSAc, PSAvar, PSA variability and expected lifespan is set, for example determined based on factors other than the PSA trend. In some embodiments, a subject's PSAgr can be categorized as low, moderate, high, or very high, and death risk calculated by using an equation appropriate for that category of PSAgr. In some embodiments, the PSA trend can be categorized as smooth or variable or as a range of variability from low to high. In some embodiments, the death risk is calculated as a death risk within a set period of time. In some non-limiting examples, the death risk is calculated as a death risk within 1 month, 2 months, 3 months, 4 months, 5 months, 6 months, 7 months, 8 months, 9 months, 10 months, 11 months, 12 months, 18 months, 2 years, 3 years, 4 years, 5 years, 6 years, 7 years, 8 years, 9 years, 10 years, 15 years, 20 years, 25 years, 30 years, 35 years, 40 years, or more than 40 years. In some embodiments, death risk is calculated based on one more prostate biomarkers or variables as described herein.

### Death Risk for a Smooth Trend

85% to 95% of the most deadly cancers produce smooth exponential growth in PSA. In general, the stronger the evidence the cancer is deadly, the higher the probability that it produced smooth exponential growth in PSA. Evidence of deadly cancer includes:
Death for BLSA men.
Progression to detection (usually by symptoms) prior to the PSA era for BLSA men.
Deadly recurrence for UCSF, Innsbruck (Tyrol), CaPSURE surgery patients (recurrence within 3 years, high PSADT, high Gleason pathology).
High risk pathology for UCSF, Innsbruck (Tyrol, AU), CaPSURE surgery patients (extra-capsular extension and high Gleason).

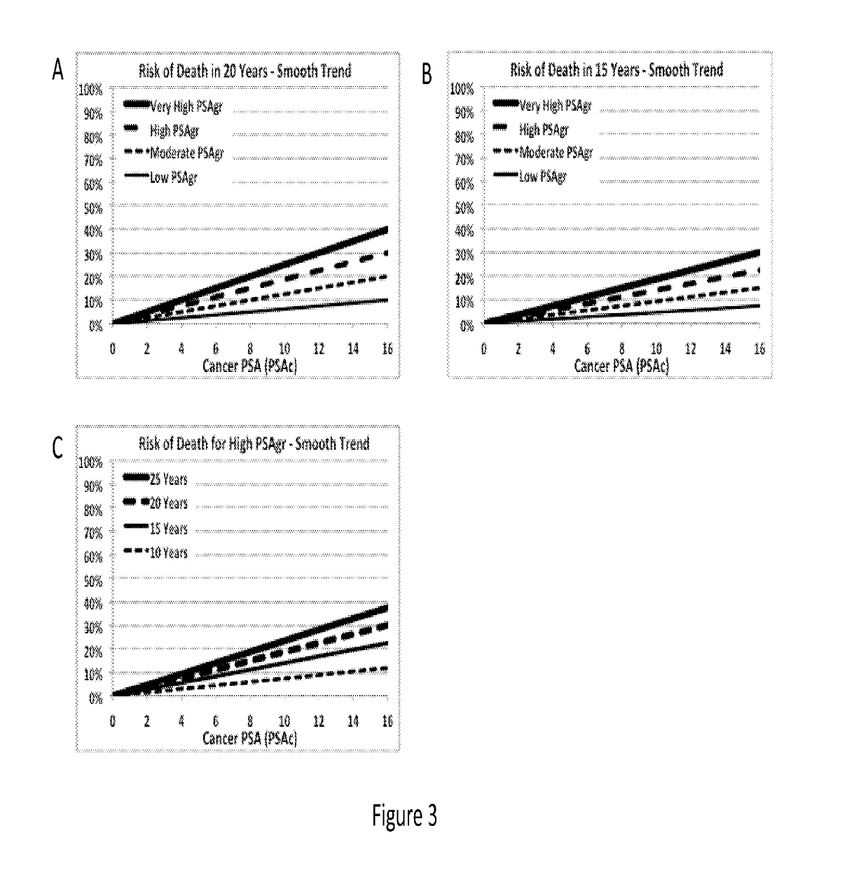

FIG. 3A shows examples of the risk of death 20 years after treatment (20 year death risk) for smooth trends with low, moderate, high, or very high PSAgr. The top thick solid black curve shows death risk for very high growth rate in cancer PSA (PSAgr). Death risk increases most steeply with increasing PSAc for very high PSAgr. In contrast, death risk increases slowest for low PSAgr, shown by the bottom thin solid black curve. The second from the top, dashed black high PSAgr curve and the third from the top, dotted black moderate PSAgr curve have slopes that fall between the two extremes.

FIG. 3B shows examples of the risk of death 15 years after treatment for smooth trends. All PSAgr curves are lower than the corresponding ones for 20 years.

FIG. 3C shows examples of the risk of death 25, 20, 15 and 10 years after treatment for smooth, High PSAgr trends. Typically, the longer the time period after treatment, the higher the risk of cancer-specific death, with the highest curve at 25 years and the lowest one at 10 years. The time period can refer to a time period after a PSA or other biomarker test, a time period after biopsy, a time period after a diagnosis of cancer, a time period after cancer treatment (such as prostatectomy), an estimated life expectancy in the absence of cancer, or any other suitable time period.

### Using Death Risk to Calculate Thresholds for Medical Action

In some embodiments, a specific death risk is set as a threshold for medical action. For example, when the death risk of a subject reaches the threshold, a medical action is performed. Medical actions include but are not limited to performing a biopsy, measuring prostate volume, treating prostatitis with antibiotics or anti-inflammatories, radiation therapy, chemotherapy, prostatectomy, ultrasound treatment, cryosurgery, hormone therapy, and treatment with pharmaceuticals. In some embodiments, for consistent screening, death risk should be the same for each screened subject (if distributions of death risks are the best information available). Unequal risk thresholds will lead to biopsies of some men with a specific death risk and no biopsies of other men with the same risk. In some embodiments, the threshold is set at a death risk of less than 1%, about 1%, about 2%, about 3%, about 4%, about 5%, about 6%, about 7%, about 8%, about 9%, about 10%, or more than 10%.

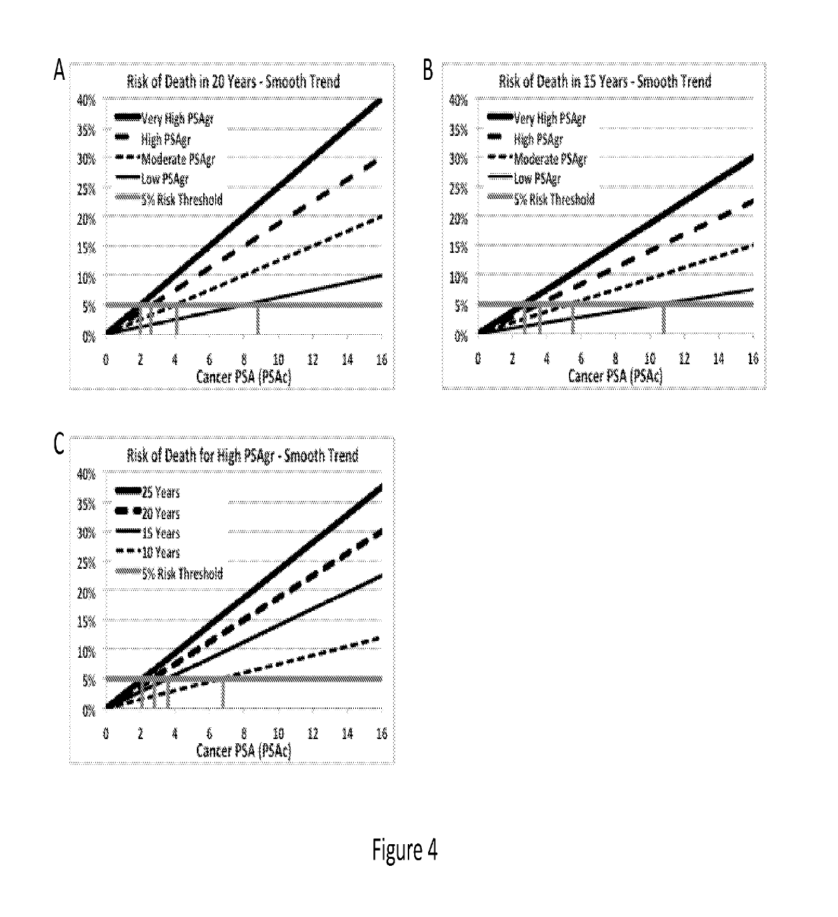

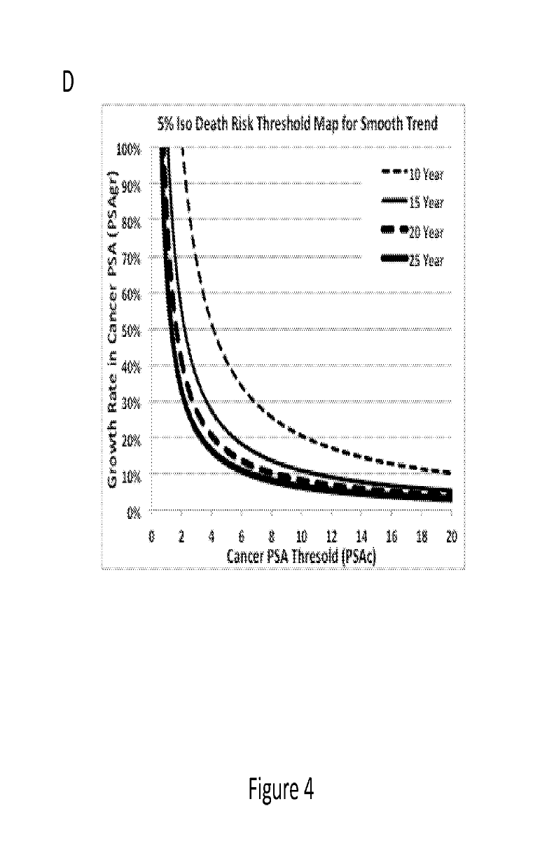

FIG. 4A depicts examples of how to determine a PSAc threshold for medical treatment when the PSA trend is smooth. FIG. 4A depicts 20 year death risks for smooth PSA trends at low, moderate, high, and very high growth rates. The horizontal gray line reflects a 5% death risk threshold. The corresponding PSAc thresholds, shown by the vertical gray lines, are very different for the four PSAgr curves. The higher PSAgr is the lower the PSAc threshold for medical action.

FIG. 4B depicts example 15 year 5% death risk thresholds for smooth PSA trends. The corresponding PSAc thresholds, shown by the vertical gray lines, are very different for the four PSAgr curves. Moreover, in this embodiment, all the PSAc thresholds are greater for 15 years, shown below, than for 20 years, shown above.

FIG. 4C compares PSAc thresholds based on a 5% death risk for 10, 15, 20, or 25 year periods based on rapidly growing, High PSAgr cancers. The time period can refer to a time period after a PSA or other biomarker test, a time period after biopsy, a time period after a diagnosis of cancer, a time period after cancer treatment (such as prostatectomy), an estimated life expectancy in the absence of cancer, or any other suitable time period. In some embodiments, the longer the time period after treatment, the higher the risk of cancer-specific death with the highest curve at 25 years and the lowest one at 10 years. In the example depicted of death after treatment, the 5% PSAc threshold is smallest, on the left, for 25 years. For men with long 25 year life expectancies, the goal is generally to catch progressing cancer very early for the most effective treatment, because even slow growing cancer might have time to kill the man. In contrast, for men with short, 10 year life expectancies, there is no need to detect and treat cancer unless it has progressed enough and produced enough PSA to threaten cancer death over the man's short expected life span.

FIG. 4D depicts constant 5% death risk curves for smooth PSA trends at 25, 20, 15 and 10 years. The thresholds are smallest (to the left) for the longest, 25 year, life expectancy. They are largest (to the right) for the shortest, 10 year, life expectancy.

### Death Risk for a Variable Trend

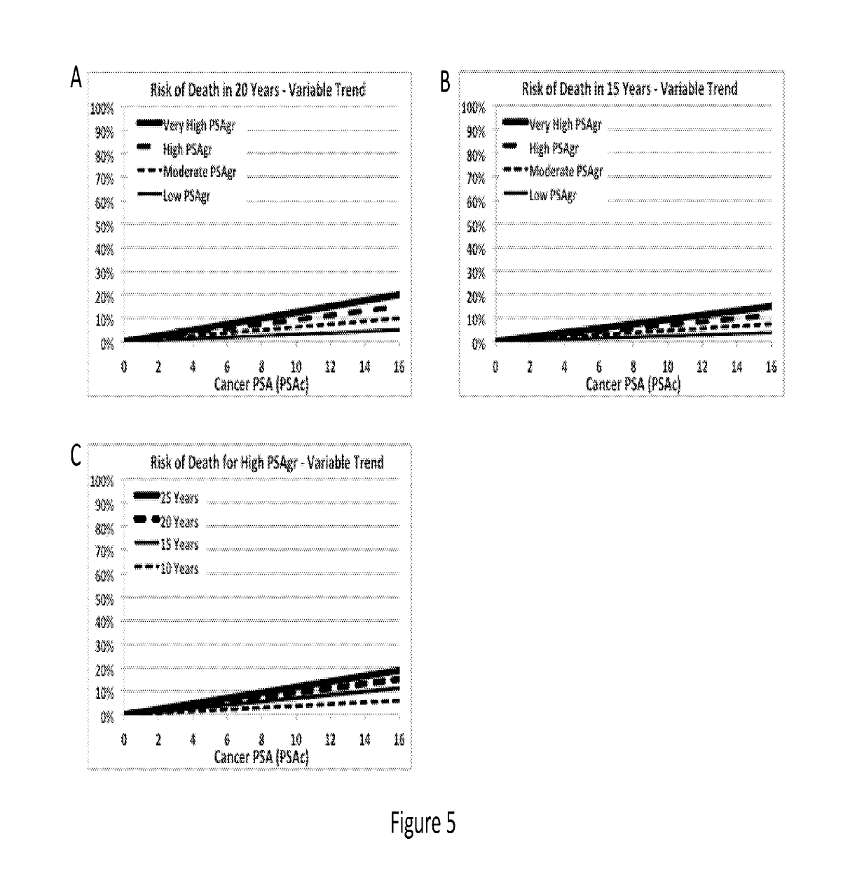

FIG. 5A shows examples of the risk of death 20 years after treatment (20 year death risk) for variable trends. The top thick solid black curve shows death risk for very high growth rate in cancer PSA (PSAgr). Death risk increases most steeply with increasing PSAC for very high PSAgr. In contrast, death risk increases slowest for low PSAgr, shown by the bottom thin solid black curve. The second from the top, dashed black high PSAgr curve and the third from the top, dotted black moderate PSAgr curve have slopes that fall between the two extremes.

FIG. 5B shows examples of the risk of death 15 years after treatment for variable trends. All PSAgr curves are lower than the corresponding ones for 20 years.

FIG. 5C shows examples of the risk of death 25, 20, 15 and 10 years after treatment for variable, High PSAgr trends. Typically, the longer the time period after treatment, the higher the risk of cancer-specific death with the highest curve at 25 years and the lowest one at 10 years.

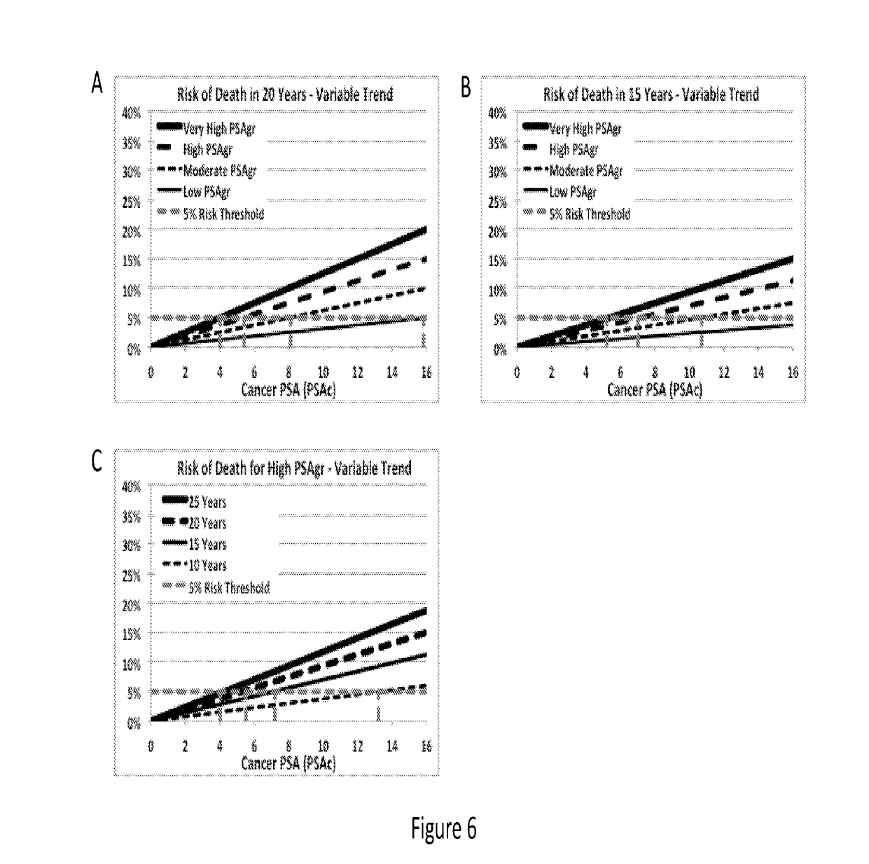

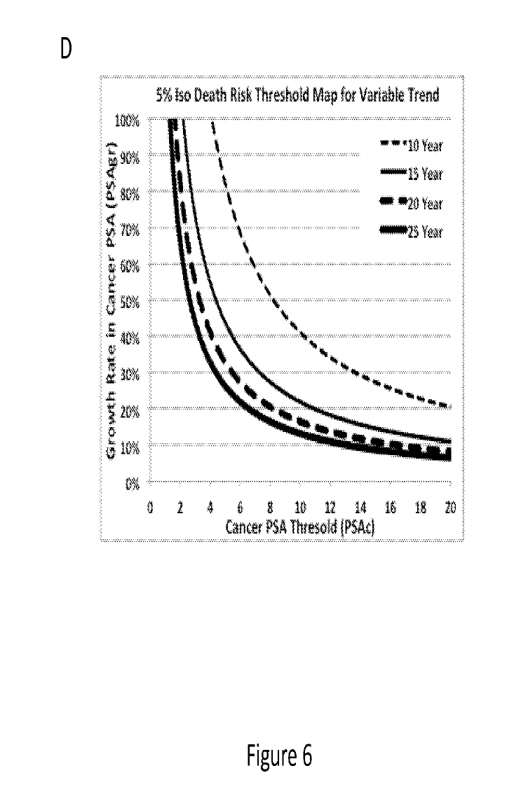

Death risks and risk thresholds for variable trends can be calculated similarly to those for smooth trends. Example thresholds are shown in FIG. 6. Typically, the risk thresholds for variable trends are at higher PSAc than for smooth trends.

### Differential Analysis

In some embodiments, differential analysis can be used with the methods of the invention. In some embodiments, multiple biomarkers and Bayesian analysis and/or Bayesian network analysis are used by the Differential Analysis system.

As described elsewhere, current screening practice using a single PSA test has three widely reported problems:
Too many unwarranted biopsies triggered by PSA elevated by some combination of BPH and prostatitis.
Over-treatment of indolent cancer inadvertently discovered by biopsies triggered by PSA elevated by some combination of BPH and prostatitis.
Excessive risk of recurrence after primary treatment because of late detection that is caused by high PSA thresholds set to reduce unwarranted biopsies and over-treatment.

Prostatitis caused by infection and inflammation is a well known cause of PSA increasing faster than volume growth. Prostatitis often can be treated effectively with antibiotics and anti-inflammatory meds. CDC studies of PSA and Free PSA ratios in the U.S. population suggest that although inflammation is widespread, infection may be the dominant benign cause of high PSAs (and therefore may be the dominant cause of fast growing PSA).

Our research with Innsbruck Medical University (Tyrol, AU) shows that benign fast growing PSA often drops substantially after a five day course of antibiotics associated with prostate biopsy that does not find cancer. Collectively these results support the strong consideration of the results of differential treatment, and suggest that a biopsy should be delayed if fast growing PSA drops or even stops growing after differential treatment.

In some embodiments, the invention improves on current practice using a differential analysis of either or both: 1) prostatitis using antibiotic and anti-inflammatory treatment, evaluated by changes from baseline for multiple biomarkers, and 2) prostate enlargement caused by BPH evaluated by ultrasound volume measurements. In some embodiments, Differential analysis is used with Dynamic analysis.

In some embodiments, Differential analysis is performed on a subject by measuring values of a prostate biomarker before, during and/or after a course of medical action. Courses of medical action include but are not limited to treatment with antibiotics, treatment with anti-inflammatories, treatment with a pharmaceutical, and ultrasound imaging. In some embodiments, biomarker values or trends measured before, during and/or after the course of medical action are compared to values or trends measured prior to the course of medical action (e.g. determining whether there is a Jump or Drop subsequent to treatment or a change in trend PSAV). In some embodiments, an observed drop after treatment for a non-cancer prostate condition suggests that the non-cancer conditions contributed to the biomarker values prior to treatment. In some embodiments, such drops increase the variability of the subject and can be used to inform later screening and risk thresholds or decisions.

Differential Analysis with PSA Only

PSA is increased by progressing cancer or by BPH and/or prostatitis. A low-cost ultrasound volume measurement can determine if the prostate is enlarged by BPH and an elevated PSA can be explained by BPH. A study by Schaeffer at Northwestern showed that treatment with antibiotics usually reduces PSA elevated by infection and often by large percentages—mean 40%. Studies by Potts at Cleveland Clinic and Bozeman at LSU have shown that treatment with antibiotics and anti-inflammatory meds reduce PSA for men with elevated PSAs by roughly 30% on average within the first month and by 40% or more over a year or more. There is evidence that antibiotics and anti-inflammatory meds reduce PSA from cancer with the possibility of prostatitis in some men by low percentages—roughly 5%. In both Potts and Bozeman unwarranted biopsies were reduced by 25% to 35% using simple decision rules.

These studies of antibiotics and anti-inflammatory treatment did not report ROC curves. However, ROC curves with AUCs in the 75% range are the natural consequence of the large difference in mean PSA drops after treatment and the width of the distributions in the articles. Also, the articles do not reveal details about the distributions of men with a mix of prostatitis and progressing cancer.

### Multiple Biomarkers

In some embodiments, multiple biomarkers as described elsewhere herein are used for differential analysis. In some embodiments, FPSA, ProPSA, or related measurements are used. FPSA % has substantially improved sensitivity and specificity compared to static PSA screening for many studies over many years. The FDA has approved its use as a way to reduce unwarranted biopsies. A recent study of ProPSA, FPSA and PSA found that FPSA % alone increased the AUC 21% points to 77% compared to 56% for static PSA alone. ProPSA % alone increased the AUC by only 1% AUC point over FPSA % alone to 78%. A logistic regression model using age, FPSA % and ProPSA % increased the AUC to 84%. These data indicate that multiple biomarkers are better than static PSA for detecting prostate cancer.

In some embodiments, a differential analysis using antibiotic and anti-inflammatory treatment along with FPSA % (and potentially ProPSA) is used to substantially increase sensitivity and specificity. An increased PSA and decreased FPSA % can indicate the presence of progressing cancer or prostate infection—but probably not BPH and/or inflammation. In some embodiments, FPSA % improves screening specificity by a limited amount because infection can mimic cancer. However, antibiotic treatment usually reduces or cures infection and reduces PSA while increasing FPSA % (revealing the prior presence of infection).

The Differential Analysis AUC using FPSA % should be much greater than the 75% to 80% using only static biomarker values (PSA, FPSA %, ProPSA %). Typical results comprise:
Low PSA (serious cancer is unlikely):
   High FPSA % (serious cancer is unlikely):
      No or Small Drop % (serious cancer is unlikely):
         Healthy prostate is likely.
Elevated PSA:
   Low FPSA %:
      No or Small Drop %:
         Serious cancer is likely.
      Large Drop % (serious cancer is unlikely):
         Infection is likely.
   High FPSA % (serious cancer is unlikely):
      No or Small Drop % (serious cancer is unlikely):
         BPH is likely and can be confirmed by volume measurement.
      Moderate Drop % (serious cancer is not very likely):
         Inflammation is likely.
      Large Drop %:
         Inflammation is likely (but this case may be unusual).

In some embodiments, subjects with serious cancer are concentrated in one combination of observed outcomes: elevated PSA, low FPSA % and no or small Drop % by differential analysis. In contrast, non-cancer conditions such as a healthy prostate, enlargement caused by BPH, inflammation or infection, are concentrated in different combinations. Infection will mimic cancer much more often than BPH and inflammation by increasing PSA and reducing FPSA %. However, in differential analysis, antibiotics and anti-inflammatory meds can reduce PSA caused by infection and increase FPSA %. FPSA % alone and differential treatment with PSA alone is useful but imperfect. The combination of the two methods can improve screening effectiveness.

### Differential Analysis—75% AUC

A 75% AUC for Differential Analysis is observed using simulations and is consistent with ROCs from simple distributions based on the published literature. The 75% AUC is based on differential analysis of prostatitis only and not BPH. Published studies have shown that low-cost ultrasound prostate volume measurements can increase AUCs.

### Dynamic Differential Analysis

In some embodiments, Dynamic Differential Analysis is used. Dynamic Differential Analysis combines Dynamic Analysis and Differential Analysis. In some embodiments, Dynamic Differential Analysis is used with no forward-looking strategy (e.g., no projection of future risks, such as recurrence or death from prostate cancer). In some embodiments, Dynamic Differential Analysis provides an 88% AUC. Embodiments of Dynamic Differential Analysis include but are not limited to Differential Jump/Drop analysis and Differential New Trend Analysis.

In some embodiments, Differential Jump/Drop analysis performs Jump/Drop analysis after or during differential treatment. In some embodiments, Differential Jump/Drop analysis comprises comparing a fitted trend with one or more biomarker values (e.g. PSA) obtained from a test performed after or during differential treatment. In some embodiments, the new fitted trend can be based at least in part on a biomarker value obtained from a test performed before differential treatment. Any of the Jump/Drop analytical methods described herein may be used. Any of the trend fitting methods and equations described herein or known in the art may be used to generate the fitted trend. In some embodiments, the trend is fitted based on all the available values of a biomarker. In some embodiments, the trend is fitted based on a subset of the biomarker values. In some embodiments, the trend is fitted to the biomarker values measured by tests performed prior to the start of differential treatment.

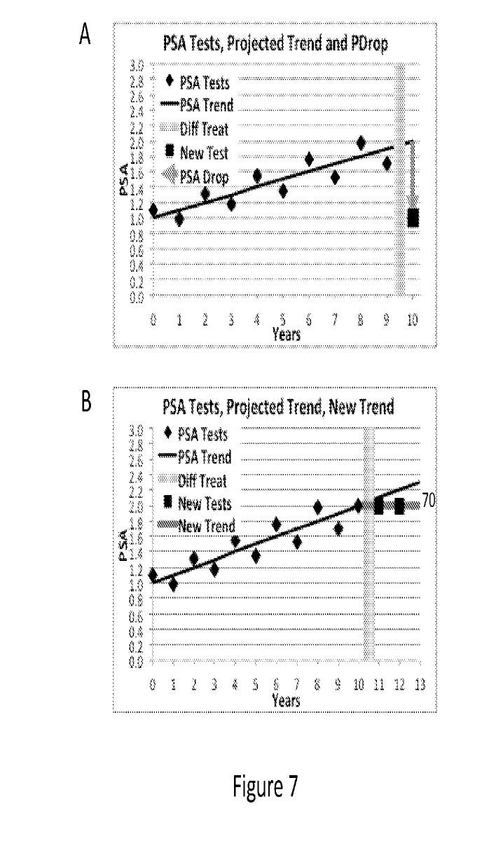

The graph in FIG. 7A shows one example of a Differential Jump/Drop analysis method. The black diamonds show PSA tests prior to the start of differential treatment. The black line shows a trend fitted to said PSA tests and projected to the date of the next test. The light gray vertical band indicates the duration of differential treatment. The black square shows a follow-up PSA test after completion of differential treatment. The vertical arrow shows PDrop, the drop in the last PSA test below the projected trend at that date, calculated as the projected PSA trend value at the time of the last test minus the last PSA test value. The percentage drop, PDrop %, is calculated as PDrop divided by the projected PSA trend value at the time of the last test. PDrop, PDrop % and other Dynamic Screening results are used to estimate the probability of various prostate conditions. In some embodiments, a PJump or a PJump % is calculated. Other suitable methods for calculating PDrop, PJump, PDrop %, and/or PJump % are described elsewhere herein.

In some embodiments, Differential New Trend analysis is performed. In some embodiments, Differential New Trend analysis compares a new fitted trend, based on one or more biomarker values (e.g. PSA) obtained from tests performed before, during and/or after differential treatment, with a previous fitted trend or its projection. In some embodiments, the new fitted trend can be based at least in part on a biomarker value obtained from a test performed before differential treatment. Any of the trend fitting methods and equations described herein or known in the art may be used to generate the new or previous fitted trend. In some embodiments, the previous trend is fitted based on all the available values of a biomarker. In some embodiments, the previous trend is fitted based on a subset of the biomarker values. In some embodiments, the previous trend is fitted to the biomarker values measured by tests performed prior to the start of differential treatment.

The graph in FIG. 7B shows one example of a Differential New Trend analysis method. The black diamonds show PSA tests prior to the start of differential treatment. The black line shows the old trend fitted to said PSA tests and projected to the date of the last PSA test. The light gray vertical band indicates the duration of differential treatment. The black squares show follow-up PSA tests after completion of differential treatment. The gray line (70) shows the new trend fitted through three PSA tests: the last test prior to the start of differential treatment and the two follow-up tests after completion of differential treatment.

Various parameters can be calculated to compare the new fitted trend with the old fitted trend. For example, Jump/Drop and Jump %/Drop % can be calculated at any point in time using the methods described herein. In some embodiments, the old trend serves as the previous, reference trend. In some embodiments, the new trend provides new values for analysis. In some embodiments, a parameter of the trends (e.g., velocity, variability, growth, PSAc, and/or acceleration) can be compared. As a non-limiting example, PSA Velocity can be calculated for the old trend (PSAVo) and for the new trend (PSAVn). In some embodiments, the PSA Velocities can be compared using a ratio PSAV %, where PSAV %=PSAVn/PSAVo.

Men with high PSAgr, such as a PSAgr of 40% or higher, are of greatest concern. In some embodiments, the high PSAgr might be caused by progressing cancer or prostatitis but almost never by BPH alone, because prostates with BPH typically grow slowly. Increasingly severe prostatitis may cause a 40% PSAgr and mimic progressing cancer, with low variation (PSAvar) exponential growth in PSA. Increasingly severe infection may be more likely to cause 40% PSAgr than increasingly severe inflammation. If infection is the cause, then the antibiotics of the Differential Analysis process are likely to cause a large drop in PSA and demonstrate that progressing cancer alone is very unlikely to be the cause of the previous 40% PSAgr trend. If inflammation (without infection) is the cause, then the anti-inflammatory meds and antibiotics (that have some anti-inflammatory properties) may not cause a noticeable drop in PSA but may flatten the new trend enough to demonstrate that progressing cancer alone is very unlikely to be the past cause of the previous 40% PSAgr trend. In some embodiments, a combination of Dynamic and Differential Analysis has a higher AUC than either Dynamic or Differential Analysis alone.

### Dynamic Differential Strategy

In some embodiments, Dynamic Differential Strategy is used. Dynamic Differential Strategy is a new approach that adds forward-looking strategy to the process that includes projecting future risks, such as recurrence after treatment and death from prostate cancer.

In some embodiments, the broad purpose of Dynamic Differential Strategy is to determine a time for a first medical action that allows time for consideration of the results of the first medical action prior to taking another (second) medical action. For example, Dynamic Differential Strategy can be used to determine a time to start differential treatment (e.g. with an antibiotic) to determine whether a subject has prostatitis due to infection prior to the subject needing a biopsy.

In some embodiments, the minimal elements of a Dynamic Differential Strategy consist of:
Obtaining a series of PSA tests.
Fitting a trend to those tests.
Determining a threshold PSA value.
Calculating a threshold time by projecting the fitted trend into the future until the trend value equals the threshold PSA value.
Determining an action lead-time.
Calculating an action time by subtracting the action lead-time from the threshold time.

In some embodiments, Dynamic Differential Strategy also includes taking medical action at the calculated action time. In some embodiments, a threshold value is determined for a second serious medical action, such as a biopsy and, should prostate cancer be detected, subsequent treatment. In some embodiments, the first, less serious medical action is differential treatment for prostatitis. Typically, the action lead-time is long enough for the consequences of differential treatment to manifest themselves, as observed in follow-up biomarker tests.

### Fit and Project a Trend

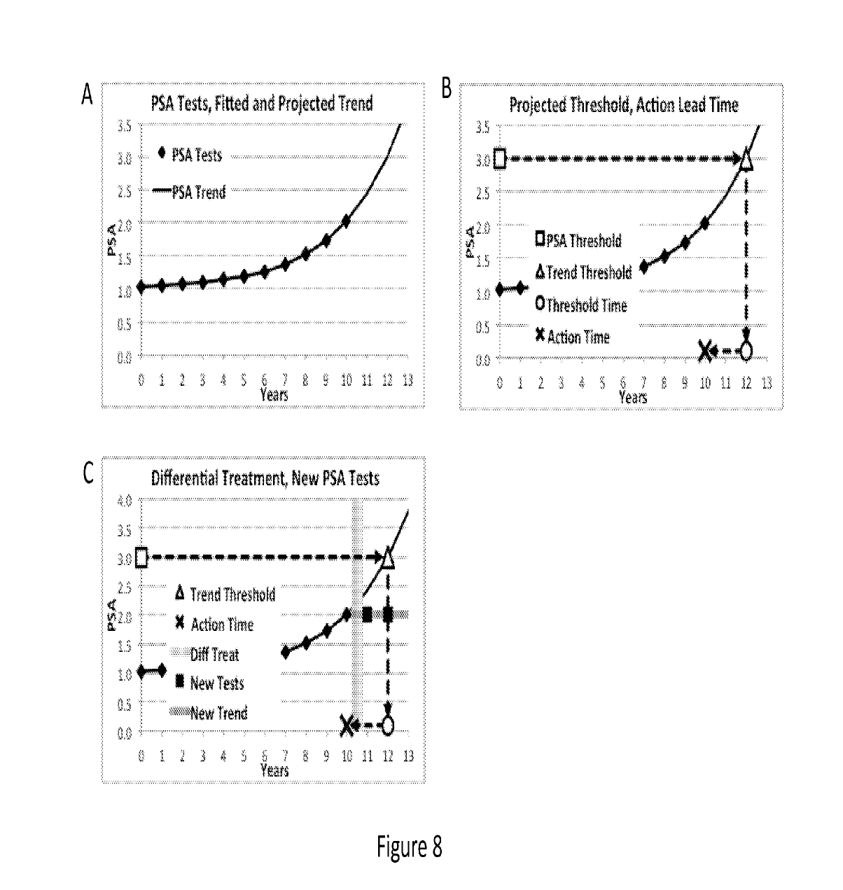

The methods disclosed elsewhere herein can be used for obtaining a series of PSA tests, fitting a trend to those tests and projecting that fitted trend into the future. In one non-limiting example, depicted in FIG. 8A, an exponential plus constant functional form is used to fit the trend.

PSA Threshold, Projected Trend Threshold and Action Time

FIG. 8B depicts an example method for determining an action time. In this example, determining a threshold PSA value is the first step, as shown by the black square at 3.0 on the y axis. The top horizontal dashed arrow shows where the projected fitted trend reaches the threshold value of 3.0, marked by the black triangle. The vertical dashed black arrow shows that, in this example, the threshold time is reached at 12 years, marked by the black circle on the x axis. The bottom horizontal arrow depicts an action lead-time of 2 years. In this example, the action lead-time is set long enough for the longer-term effects of differential treatment to manifest themselves, including flattening or depressing the PSA trend, in light of information about the man and Dynamic Screening variables, such as PSAgr. The final step is to calculate the action time, by subtracting the action lead-time from the threshold time. In this example, the action time, as shown by the black X, is at 10 years, which is equal to the threshold time of 12 years minus the action lead-time of 2 years.

### Differential Treatment, New PSA Tests and Trend Analysis

The action lead-time can be used to trigger medical action, such as differential treatment for prostatitis shown by the vertical light gray bar in FIG. 8C. In some embodiments, Dynamic Differential Analysis methods as disclosed herein can be used to trigger follow-up PSA tests and their analysis, including but not limited to using previously disclosed Differential Jump/Drop Analysis and Differential New Trend Analysis. The horizontal gray line shows a new fitted trend through the last three PSA tests, which can be compared to the projected old trend.

Determining a PSA Threshold Value and/or Threshold Time

In some embodiments, for any fitted trend, the PSA threshold value and the threshold time are equivalent—e.g., for each PSA threshold value there is a unique corresponding threshold time. In the fitted trend example of FIG. 8, the threshold time of 12.0 years corresponds uniquely to the 3.0 PSA threshold value.

In some embodiments, an optimization process is used to determine a preferred time for biopsy and subsequent treatment if prostate cancer is detected. In some embodiments, one or more risks/costs, including but not limited to a risk or cost of biopsy, a risk or cost of treatment, and/or risk or cost of a prostate cancer outcome (such as recurrence or death) are projected, weighted, and summed. In some embodiments, the risk/cost is projected with the help of a fitted PSA trend. In some embodiments, the risk/cost is weighted based on patient preference, life expectancy, and/or other health conditions. In some embodiments, the preferred time for biopsy is the minimum of the summed risks/costs.

In some embodiments, the risks/costs are projected based on the assumption that the results of certain differential actions are negative with respect to prostate cancer, and the absence of differential responses increases the estimated probability of cancer. In some embodiments, differential treatment for prostatitis is assumed to have no effect on future PSA tests, which fall on the projected fitted trend. In some embodiments, a differential measurement of prostate volume is assumed to find a normal, or relatively small, prostate. These assumptions decrease the risk that the actual optimal time for biopsy, as calculated after analyzing the results for differential treatment, is sooner than the projected optimal time for biopsy, calculated prior to performing differential medical action. Thus, the assumptions err on the side of early projected biopsy timing so that differential treatment and analysis does not prevent the subject from getting a biopsy in time for effective treatment.

### Determining an Action Lead-Time

In some embodiments, the action lead-time is set to be long enough for the consequences of medical actions to manifest themselves prior to a required time or deadline for making a decision about subsequent medial actions. Typically, differential medical actions are the first medical actions taken after an initial series of PSA tests because they are generally low-impact and low-cost. Typically, biopsy and subsequent treatment for prostate cancer are the later medical actions taken because they are more likely to risk high negative impact and high cost. Generally, high-impact and/or high-cost actions should be deferred until low-cost evidence is gathered and analyzed. Differential medical actions include but are not limited to differential treatment for prostatitis, analysis of follow-up PSA tests after differential treatment, and differential measurement of prostate volume, such as by using ultrasound imaging. Of these medical actions, differential treatment typically requires the longest time for follow-up and analysis, so we will focus on describing example methods for calculating its action lead-time. Similar methods can be used to calculate action lead-time for other differential medical actions.

In some embodiments, the action lead-time for differential treatment can be as long as several years or more. In some embodiments, selecting the action lead-time depends on any of a variety of factors, including but not limited to PSAgr, PSAc, PSAvar and other measure of PSA variability, age, life expectancy, personal preferences and/or others. In the following non-limiting examples, we focus on the estimated exponential growth rate in cancer PSA (PSAgr). We consider two example cases: high PSAgr and low PSAgr.

In some embodiments, the differential treatment medical lead-time selected for high PSAgr is relatively short. Reasons for this include but are not limited to:
The PSAc threshold (and corresponding PSA threshold) is relatively low for high PSAgr trends because the cancers are likely to be deadly and hard to treat.
PSA grows very quickly for high PSAgr trends. There is relatively little time between a noticeable increase in PSA and the relatively low PSAc threshold.
High PSAgr prostatitis is often caused by infection, which often responds quickly to differential treatment with antibiotics. PSA may drop or the trend may flatten out to provide rapid evidence that deadly cancer is unlikely to have been the cause of the previously increasing PSA.
   In some embodiments, the differential treatment medical lead-time selected for low PSAgr is relatively long. Reasons for this include but are not limited to:
The PSAc threshold (and corresponding PSA threshold) is relatively high for low PSAgr trends because the cancers are not likely to be deadly and hard to treat.
PSA grows slowly for low PSAgr trends. There is a relatively long time between a noticeable increase in PSA and the relatively high PSAc threshold—years or even a decade for very low PSAgr.
Low PSAgr prostatitis is often caused by inflammation without infection, which often responds slowly to differential treatment with anti-inflammatory medications and/or antibiotics. PSA may not drop and only the trend may flatten out to provide gradual evidence that deadly cancer is unlikely to have been the cause of the previously increasing PSA. It may take years to become confident that the new trend has flattened out compared to the previously slowly growing PSA trend.

In some embodiments, Dynamic Differential Strategy consists of a minimum of:
Projecting PSA trends into the future.
Projecting one or more prostate cancer related risks into the future for one or more medical actions or outcomes, such as biopsy, treatment, recurrence and death from prostate cancer.
Taking or delaying current medical action based on the projections.

In some embodiments, Dynamic Differential Strategy uses one or more of the following elements:
An optimization process that balances:
   Risk of recurrence after treatment.
      If cancer
      Overall, weighted by the risk of cancer.
   Chance of biopsy (or unwarranted biopsy).
   Chance of treatment (or over-treatment).
Projection of PSA and other biomarker trends.
Anticipation of a biopsy in the future if trends continue, based on a “what if” optimization process for the projected trends where negative outcomes are assumed for differential processes for prostatitis and BPH (enlarged prostate).
Early differential process (perhaps several years before the anticipated “what if” biopsy) that includes volume measurement for BPH and treatment for prostatitis with antibiotics and/or anti-inflammatory medications. PDP is our short hand for this Prostatitis Detection Process.
Monitoring of biomarker trends after treatment.
Final differential process when a biopsy seems warranted soon by this anticipation process. In some embodiments, the final differential process consists of another differential treatment for prostatitis and/or prostate volume measurement to confirm the elevated probability of progressing cancer prior to making a biopsy decision.
Biopsy, if warranted, or continued treatment for prostatitis.

In some embodiments, a “what if” optimization process is used prior to taking certain screening actions, such as differential treatment for prostatitis or differential measurements of prostate volume. For example, we are interested in how soon a biopsy and subsequent treatment for prostate cancer might be warranted based on projections of the current PSA trend. However, in some embodiments biopsy timing will depend on the results of the screening action(s). For example, differential treatment for prostatitis might have no impact on subsequent PSA tests that follow the projected trend, or differential treatment might decrease PSA and invalidate the projected trend. In some embodiments, if no impact is observed, a biopsy might be warranted soon. In some embodiments, if PSA decreases, a biopsy might not be warranted in the foreseeable future. In some embodiments “what if” analysis is used to identify when an early biopsy might be warranted. In some embodiments, “what if” analysis comprises assuming that differential treatment has no impact on the projected PSA trend, and using the optimization process to predict when a biopsy might be warranted under this assumption.

In some embodiments, a Dynamic Differential Strategy uses all the evidence to maximum advantage. In some embodiments, Dynamic Differential Strategy has an AUC of 95%. In some embodiments, Dynamic Analysis is used to project current trends into the future to anticipate when a biopsy might be warranted by the evidence if cancer is actually progressing. In some embodiments, the Differential Analysis process is triggered several years earlier than when the biopsy might be warranted, in order to assess the effect of antibiotic and anti-inflammatory treatment over several years.

Dynamic Analysis of Innsbruck, UCSF and CaPSURE data shows that progressing cancer PSA trends seldom decelerate. This implies that PSA from progressing cancer usually will continue at the same growth rate after antibiotic and anti-inflammatory treatment, or close to the past growth rate. For example, at a 40% growth rate, PSA from cancer will roughly double over two years. With Dynamic follow-up, antibiotics and anti-inflammatory medications only need to slow the growth in PSA in order to show that progressing cancer is unlikely to have been the dominant cause of the 40% previous growth in PSA. In addition, PSA from prostatitis typically decreases more for longer observation periods and also for longer periods of treatment. PSA will likely keep growing at a high rate if cancer is the major cause, and will slow or often decrease if prostatitis is the major cause. Therefore, waiting several years or more after one or more treatments for prostatitis substantially increases specificity compared to simply considering how much PSA drops soon after treatment.

The estimated 95% AUC for Dynamic Differential Strategy was calculated based on simulations. In one non-limiting example, a simple simulator of a large population of men was constructed based on published evidence regarding the distribution of cancer, its static and dynamic characteristics, and the results of antibiotic and anti-inflammatory treatment. Without being limited by any theory, the intuition behind the result is that typical progressing cancer is different from typical BPH and/or prostatitis on many dimensions. Three are considered below. Strategy increases the power of PSA Drop % compared to the Dynamic Differential Analysis described elsewhere herein that does not project, anticipate and monitor differential outcomes for several years:
PSA Growth Rate (PSAgr):
   Cancer: Higher
   Prostatitis: Lower
   BPH: Very low
PSA Variation (PSAvar):
   Cancer: Lower
   Prostatitis: Higher
   BPH: Low but matters little because of very low PSAgr.
PSA Drop % after Antibiotic and Anti-Inflammatory Treatment:
   Cancer: Substantial INCREASE (not a drop) based on PSAgr.
   Prostatitis: Large initial DROP that is likely to grow in size.
   BPH: Low but matters little because of very low PSAgr.

A Dynamic Differential Strategy using only PSA is expected to produce an AUC greater than 95%. In some embodiments, the Dynamic Differential Strategy AUC is expected to increase with the use of FPSA. However, the increase will be moderated by diminishing returns.

### Dynamic Differential Strategy System

In some embodiments, the system projects PSA trends and analyzes “what if” scenarios to determine the optimal time for actions. In some embodiments, optimal timing balances the risk of recurrence, e.g. Recurrence % (R %), against Weighted Unwarranted Biopsy % (UB %). In some embodiments, the weighting process considers risk of over-treatment. In some embodiments, the patient and doctor assess the cost of Weighted Unwarranted Biopsies compared to the risk of recurrence. Total Risk % can be calculated as a sum of the two risks.

The subsections below outline examples of how a strategy process might unfold over time for a man with progressing cancer. The gray shaded area represents the past and the white unshaded area represents the future that the system periodically projects and analyzes.

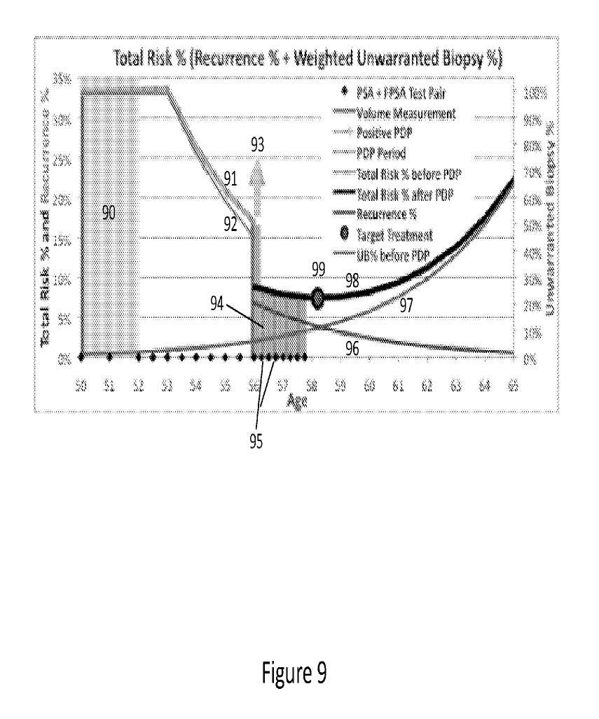

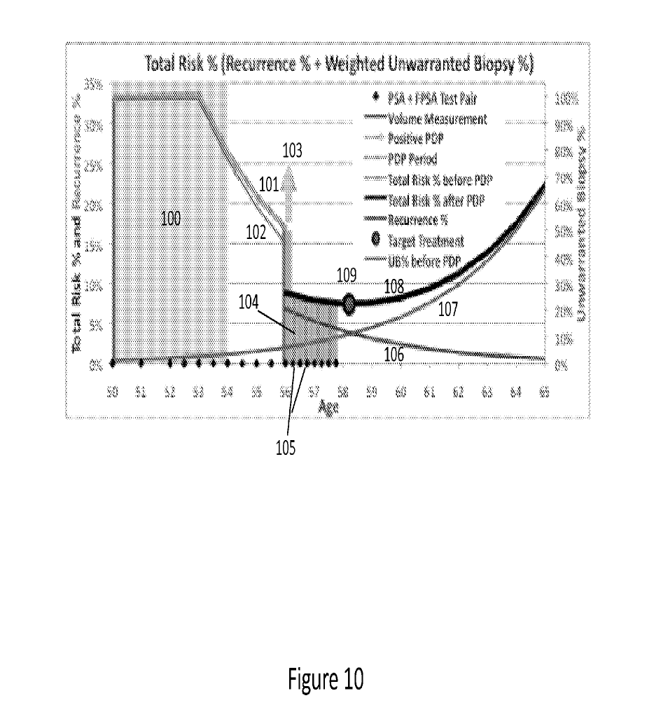

FIG. 9 depicts an example of a strategy process for a subject at age 52. In this example, PSA trends are increasing at age 52 (not shown). The gray shaded area (90) represents the past. The dark gray curve (91) shows the Total Risk % before the Prostatitis Detection Process (PDP). It is the sum of the risk of unwarranted biopsy (UB %) and risk of recurrence (Recurrence %) curves before PDP. The lighter gray curve (92) shows the UB % before PDP. The vertical gray arrow (93) shows the direction that the Total Risk % will move if the PDP finds evidence of prostatitis. The dark gray area (94) shows the PDP period from age 56 to nearly age 58 when prostatitis is treated and/or follow-up PSA tests are monitored. The system suggests more frequent PSA tests (95) at various times based on analysis. The vertical gray lines at age 50 and 56 mark times of prostate Volume Measurements. The gray curve near the bottom on the right (96) shows the Unwanted Biopsy % (UB %) after a negative PDP that finds no evidence of prostatitis. The low gray curve (97) shows the Recurrence %. The thick black curve (98) shows the Total Risk % after a negative PDP that finds no evidence of prostatitis. The black circle (99) shows the target time for prior biopsy and, if cancer is detected, treatment at the point of minimum Total Risk %. FIGS. 10, 11 and 12 follow the same numbering conventions where, for example, 90, 100, 110 and 120 refer to the past that increases in width for each subsequent Figure.

FIG. 10 depicts an example of the strategy process for the same subject, now at age 54, where the PSA trend continues to increase. The system suggests more frequent testing should continue (105). The gray shaded area (100) represents the past. The dark gray curve (101) shows the Total Risk % before the Prostatitis Detection Process (PDP). It is the sum of the risk of unwarranted biopsy (UB %) and risk of recurrence (Recurrence %) curves before PDP. The lighter gray curve (102) shows the UB % before PDP. The vertical gray arrow (103) shows the direction that the Total Risk % will move if the PDP finds evidence of prostatitis. The dark gray area (104) shows the PDP period when prostatitis is treated and/or follow-up PSA tests are monitored. The system suggests more frequent PSA tests (105) at various times based on analysis. The vertical gray lines at age 50 and 56 mark times of prostate Volume Measurements. The gray curve near the bottom on the right (106) shows the Unwanted Biopsy % (UB %) after a negative PDP that finds no evidence of prostatitis. The low gray curve (107) shows the Recurrence %. The thick black curve (108) shows the Total Risk % after a negative PDP that finds no evidence of prostatitis. The black circle (109) shows the target time for prior biopsy and, if cancer is detected, treatment at the point of minimum Total Risk %.

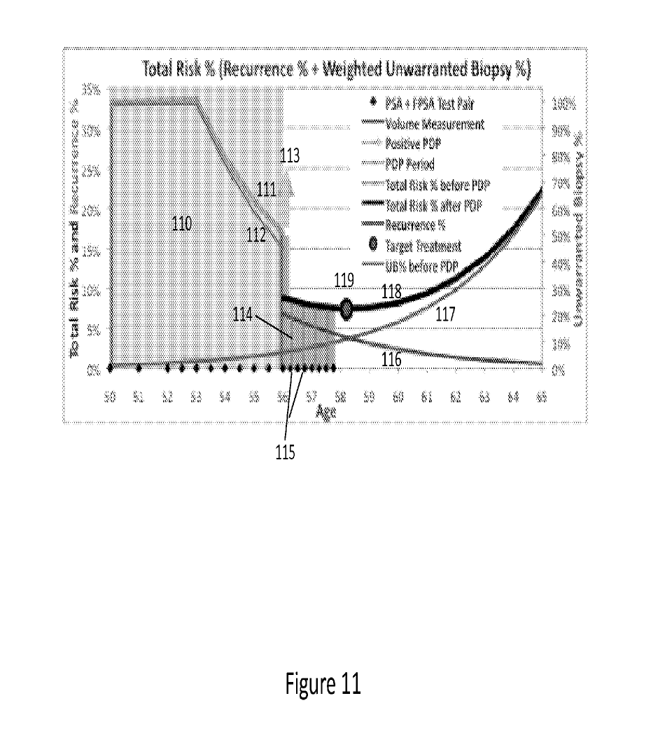

FIG. 11 depicts an example strategy process for the subject at age 56 (shaded area 110), where PSA has increased enough (and FPSA % has decreased enough) to anticipate a biopsy (and treatment, if warranted) just beyond age 58 based on projections of PSA and FPSA trends. A differential process is now suggested over two years before the anticipated “what if” biopsy. The volume measurement reveals a prostate of normal size (not enlarged that might account for an elevated PSA). The prostatitis differential process is negative. Treatment with antibiotics and anti-inflammatory meds does not decrease PSA. The system suggests very frequent monitoring of PSA and FPSA for the next two years (115).

Progressing cancer is considered in this example. However, if the volume measurement at age 56 showed an enlarged prostate and the prostatitis process was positive (and suggested infection and/or inflammation), then risk of unwarranted biopsy would increase sharply, as suggested by the vertical gray arrow (113). In such a case, the system would encourage treatment of BPH and/or prostatitis and reduce the alert level for progressing cancer.

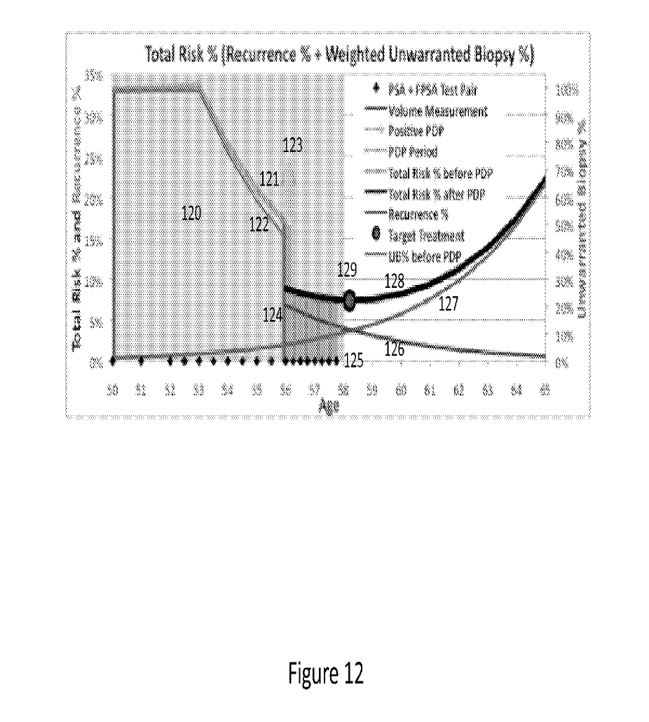

FIG. 12 depicts an example strategy process for the subject aged late 57 and early 58, where PSA and FPSA trends after differential treatment suggest an increasing risk of progressing cancer. The patient and doctor may elect a second differential process for prostatitis (PDP) and perhaps another volume measurement. If the results continue to be negative (no evidence of prostatitis and enlargement) the optimization system will suggest/show that it is time for a biopsy and, if warranted, treatment.

### Two Variable Optimization

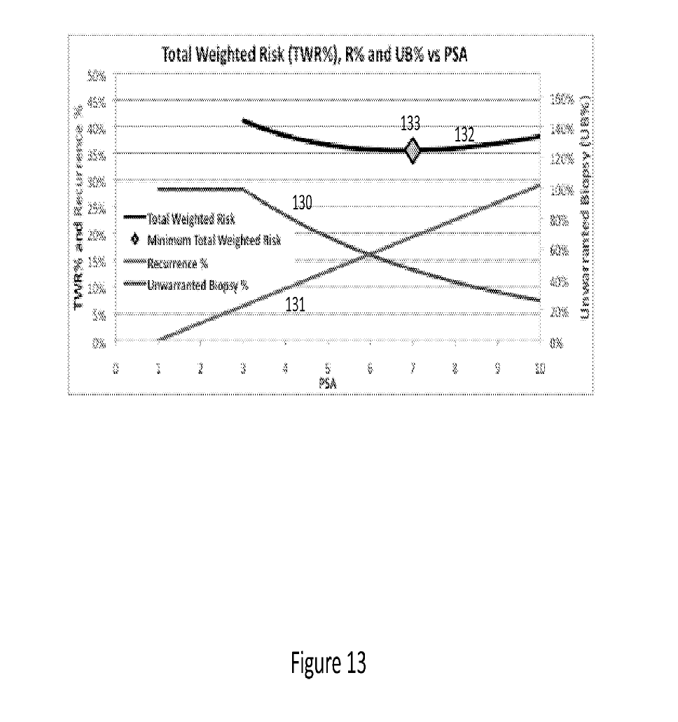

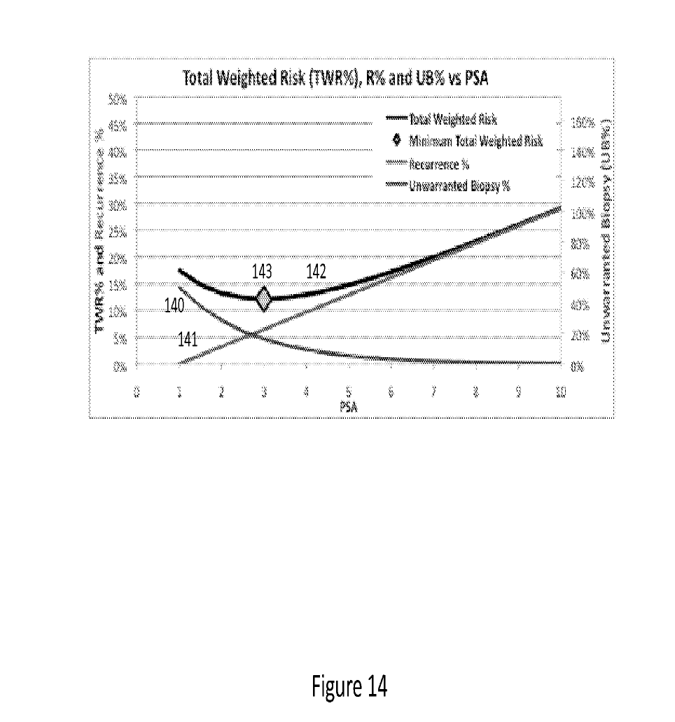

In some embodiments, Total Weighted Risk (TWR %) (132 and 142 on FIGS. 13 and 14) is the sum of two risks/costs, such as Recurrence Risk (R %) (131 and 141) and weighted Unwarranted Biopsy risk (UB %) (130 and 140). The weight might increase UB % to account for the additional related risk of over-treatment. The weight will decrease UB % to account for its lower personal cost than recurrence in the opinion of the man making the decision. In this example, weighted UB % is about one-third as “costly” as R %, as depicted by the different scales used on the graph (100% UB %, on the right scale, corresponds to roughly 30% R %, on the left scale.)

Static PSA Screening—The poor screening effectiveness of static PSA screening leads to a high risk of unwarranted biopsies (130), as shown in FIG. 13. Minimum TWR % (133) is reached at a relatively high PSA of about 7.0.

Dynamic Differential Strategy—Full Dynamic Differential Strategy substantially reduces UB % (140), as shown in FIG. 14. Minimum TWR % (143) is reduced from 35% above to roughly 12% below. The optimal PSA for biopsy is reduced from 7.0 to 3.0, which results in a reduction in risk of recurrence from about 20% to about 7%.

### Three Variable Optimization

In some embodiments, another approach to optimization is used that considers three variables:
Overall risk of recurrence (weighted by risk of cancer).
Chance of biopsy.
Chance of treatment.

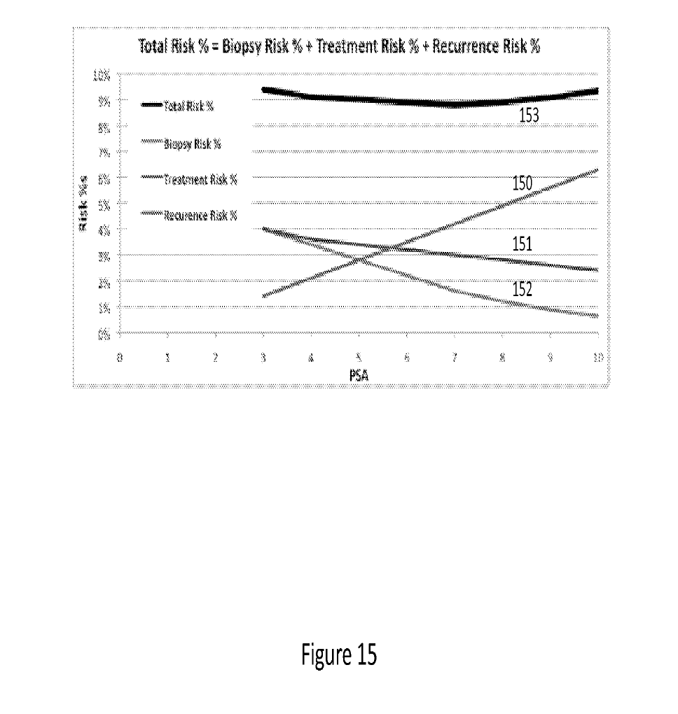

Total Risk %—FIG. 15 depicts one example of how the breakdown of total risk varies with the PSA value. Total Risk % (153) is equal to Biopsy Risk % (BR %) (152) plus Treatment Risk % (TR %) (151) plus Recurrence Risk % (RR %) (150). BR % and TR % are weighted relative to RR % by the man and his doctor based on their assessment of relative costs. FIGS. 15, 21, 22, 23 and 24 follow the same numbering conventions where, for example, 153, 213, 223, 233 and 243 refer to Total Risk % for each Figure.

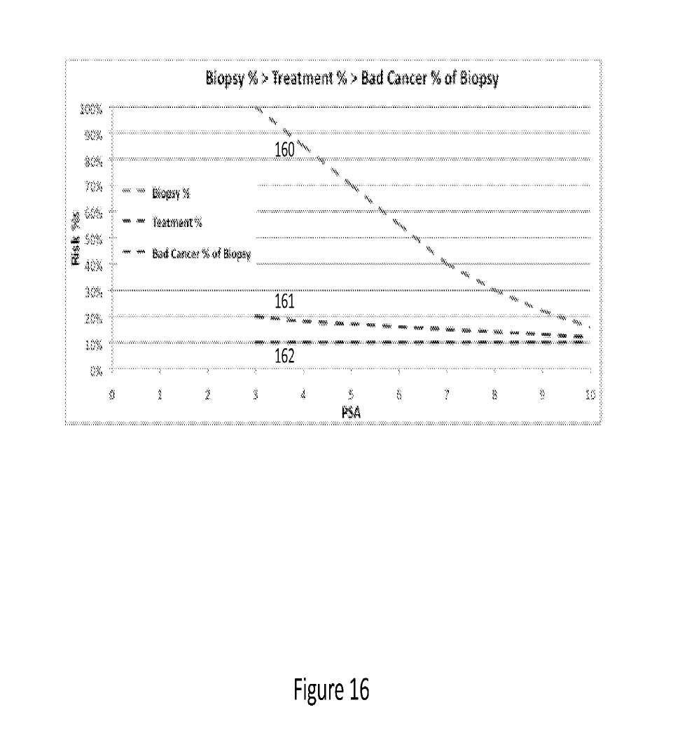

Hierarchy—FIG. 16 shows some non-limiting examples of key variables that affect risk and their risk hierarchy: Biopsy % (160), Treatment % (161) and Bad Cancer % of Biopsy (162). They are described in greater detail below.

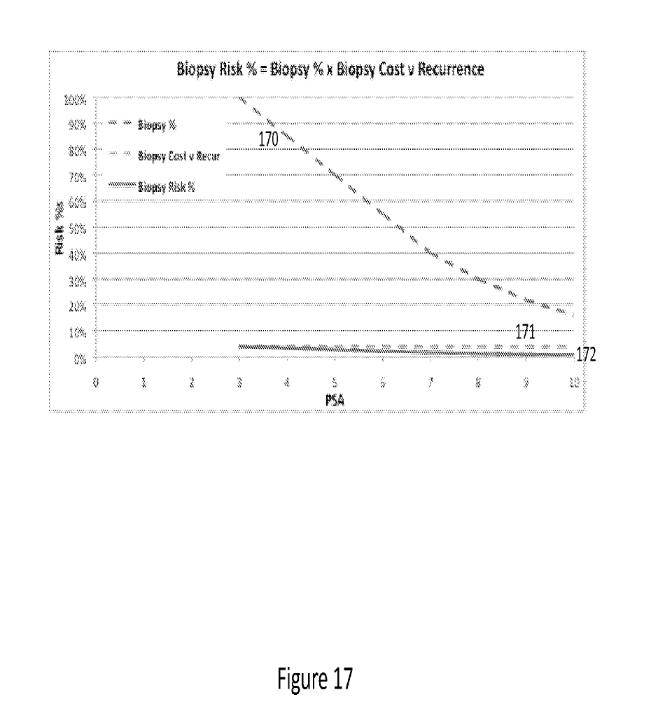

Biopsy Risk %—FIG. 17 depicts an example set of curves that plot Biopsy %, Biopsy Cost vs Recurrence, and Biopsy Risk % vs PSA. Biopsy Risk % (172) is the product of two variables:
Biopsy % (170), which is the probability a biopsy will be warranted.
Biopsy Cost vs Recurrence (171), which is a discount factor based on the man's assessment of the personal cost of biopsy vs recurrence. In some embodiments, one or both of Biopsy % and Biopsy Cost vs Recurrence can vary with an observed prostate biomarker, such as observed PSA. In some embodiments, the relationship between Biopsy % and/or Biopsy Cost vs Recurrence is based on personal characteristics of the subject.

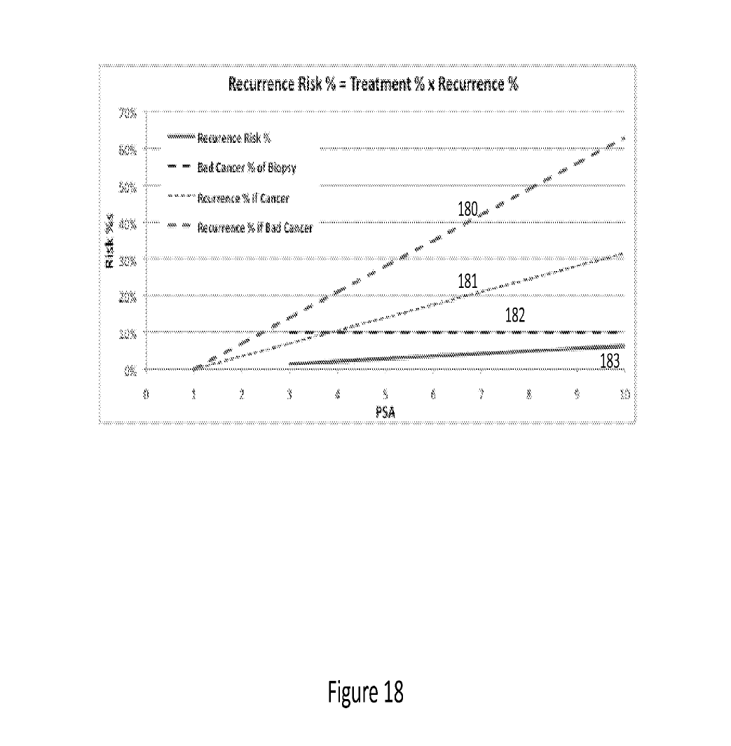

Recurrence Risk %—FIG. 18 depicts an example set of curves that plot Treatment %, Recurrence %, and Recurrence Risk % vs PSA. Recurrence Risk % (183) is the product of two variables:
Treatment %, which is the risk that treatment will be warranted after biopsy. It may be equal to the risk of finding cancer by biopsy, or nearly equal to that risk.
Recurrence % (181) is the risk of recurrence if cancer is found by biopsy. In some embodiments, one or both of Treatment % and Recurrence % can vary with an observed prostate biomarker, such as observed PSA. In some embodiments, the relationship between Treatment % and/or Recurrence % is based on personal characteristics of the subject.

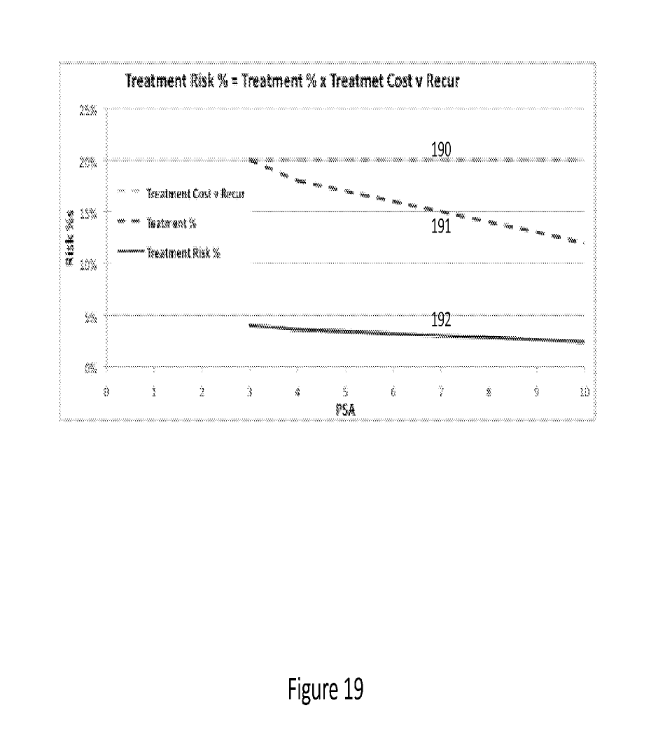

Treatment Risk %—FIG. 19 depicts an example set of curves that plot Treatment % (191), Treatment Cost vs Recur (190), and Treatment Risk % (192) vs PSA. Treatment Risk % is the product of two variables:
Treatment % is the risk that treatment will be warranted after biopsy, as above.
Treatment Cost vs Recur, which is a discount factor based on the man's assessment of the personal cost of treatment vs recurrence. In some embodiments, one or both of Treatment % and Treatment Cost vs Recur can vary with an observed prostate biomarker, such as observed PSA. In some embodiments, the relationship between Treatment % and/or Treatment Cost vs Recur is based on personal characteristics of the subject.

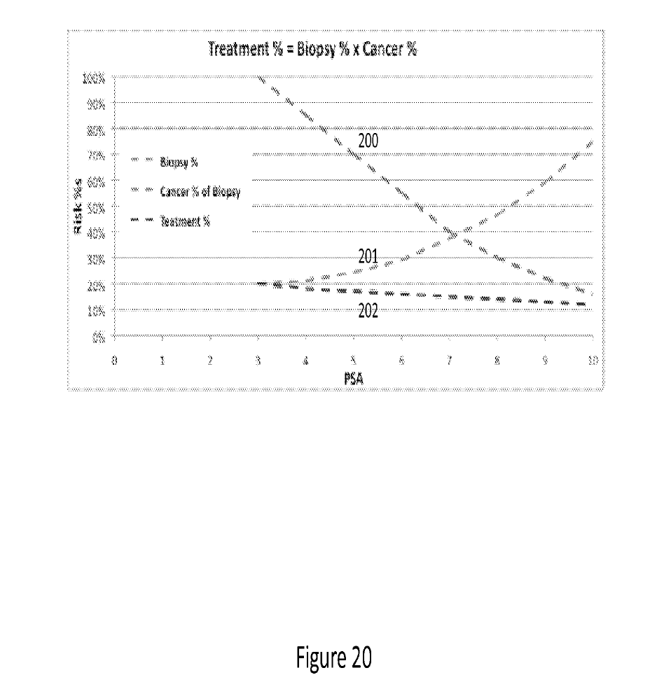

Treatment %—FIG. 20 depicts an example set of curves that plot Biopsy % (200), Cancer % (201), and Treatment % (202) vs PSA. Treatment % is the product of two variables:
Biopsy %, as defined above.
Cancer %, which is the probability that cancer is found by biopsy. In some embodiments, one or both of Biopsy % and Cancer % can vary with an observed prostate biomarker, such as observed PSA. In some embodiments, the relationship between Biopsy % and/or Cancer % is based on personal characteristics of the subject.

Strategic Use of Three Variable Optimization for Static PSA

The following non-limiting examples show the strategic use of three variable optimization for static PSA screening—that is not very effective. PSA is assumed to continue increasing due to progressing cancer. Three scenarios are shown, for PSA values of 3.0, 5.0, and 7.0.

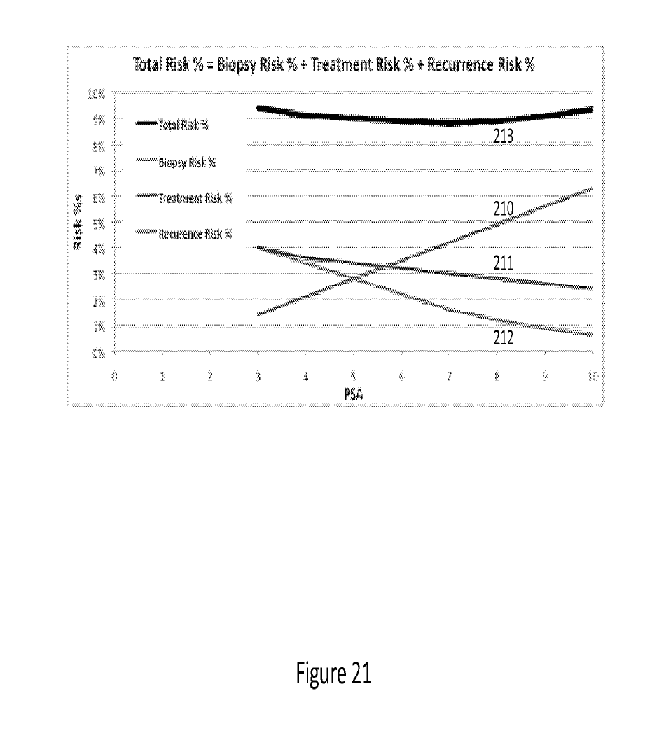

3.0 PSA—FIG. 21 shows a projected scenario for a subject using static PSA screening. In this example, total Risk % (213) is estimated to decrease as PSA increases until reaching a PSA value of about 7.0. Biopsy Risk % (BR %) (212), Treatment Risk % (TR %) (211), and Recurrence Risk % (RR %) (210) are also depicted.

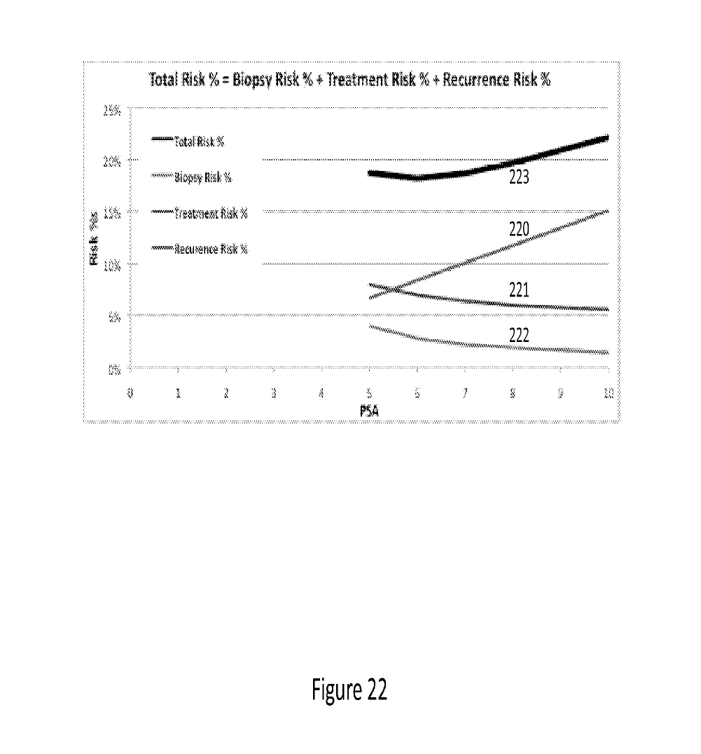

5.0 PSA—FIG. 22 shows a projected scenario for a subject using static PSA screening. In this example, total Risk % (223) is estimated to decrease as PSA increases until reaching a PSA value of about 6.0. Biopsy Risk % (BR %) (222), Treatment Risk % (TR %) (221), and Recurrence Risk % (RR %) (220) are also depicted.

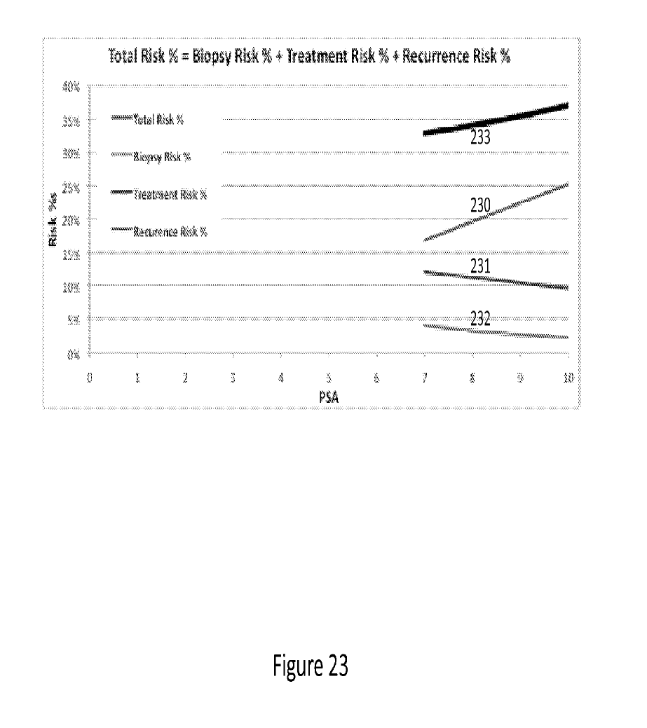

7.0 PSA—FIG. 23 shows a projected scenario for a subject using static PSA screening. In this example, projected scenarios estimate that Total Risk % (233) will increase as PSA increases above 7.0. In this example, a biopsy is overdue. Biopsy Risk % (BR %) (232), Treatment Risk % (TR %) (231), and Recurrence Risk % (RR %) (230) are also depicted.

### Use of 3 Variable Optimization for Dynamic Differential Strategy

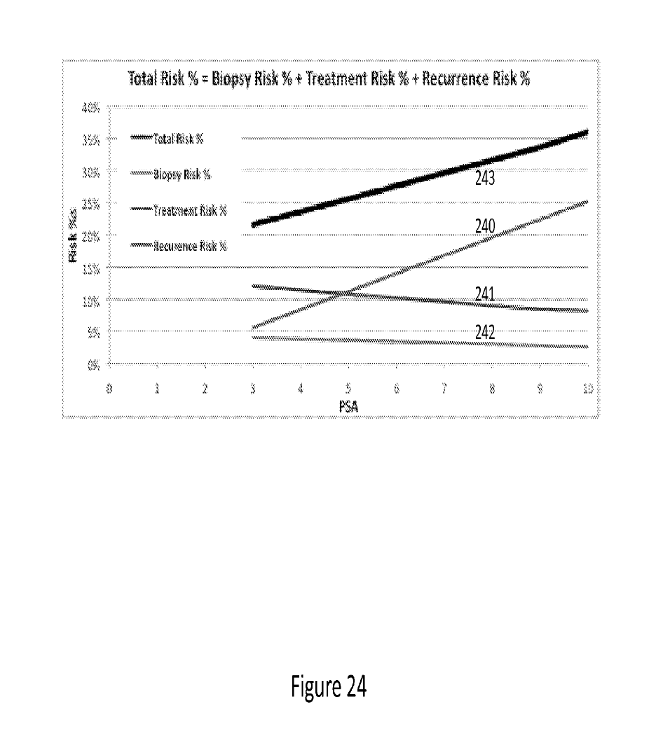

Screening using a Dynamic Differential Strategy (DDS) will lead to optimal biopsies at much lower PSA (for early detection and effective treatment) than static PSA screening. FIG. 24 depicts an example projection based on DDS for a subject with a PSA of 3.0. In this example, an optimal biopsy is already overdue because Total Risk % (243) increases as PSA increases above 3.0. A full DDS starting at much lower PSAs would have led to an optimal biopsy at PSA less than 3.0. Biopsy Risk % (BR %) (242), Treatment Risk % (TR %) (241), and Recurrence Risk % (RR %) (240) are also depicted.

### Performance Evaluation

The methods described herein can be used to screen for high-risk prostate cancers, such as rapidly growing cancers. In some embodiments, performance of the methods can be further evaluated or optimized by determining how effectively the methods of the invention detect high-risk prostate cancer. In some embodiments, static PSA Screening and Dynamic Analysis ROC curves and AUC performance are estimated directly from retrospective analysis, such as by using the methods presented in Example 1. ROC refers to a graphical representation of sensitivity as a function of 1-specificity for various possible threshold values or analysis methods. Sensitivity typically refers to the proportion of detected true positive cases, while 1-specificity refers to the proportion of false positive cases. Specificity can be calculated as a ratio of the number of true negatives detected over the sum of the true negatives and false positives detected. Sensitivity can be calculated as a ratio of the number of true positives detected over the sum of the true positives and false negatives detected. AUC refers to the area under an ROC curve, and represents the probability that a positive test (e.g. need for a biopsy or detection of cancer) is classified as positive by the analysis method. In some embodiments, the AUC of the method is at least 0.5. In some embodiments, the AUC of the method is at least 0.6. In some embodiments, the AUC of the method is at least 0.7. In some embodiments, the AUC of the method is at least 0.8. In some embodiments, the AUC of the method is at least 0.85. In some embodiments, the AUC of the method is at least 0.87. In some embodiments, the AUC of the method is at least 0.9. In some embodiments, the AUC of the method is at least 0.92. In some embodiments, the AUC of the method is at least 0.95. In some embodiments, the AUC of the method is at least 0.98.

In some embodiments, Differential Analysis performance is estimated using analysis of data published in medical journal articles.

In some embodiments, high-risk prostate cancers are identified using one or more criteria, including but not limited to Gleason score, clinical stage (e.g. as established by the American Joint Committee on Cancer), tumor size, and other cancer endpoints, such as death from prostate cancer, metastasis, recurrence after treatment and its severity (measured by time to recurrence, PSA doubling time at recurrence and pathology Gleason score if available). In some embodiments, serious cancer can be detected by additional tests, including but not limited to additional biomarker tests, biopsies, or cancer mortality.

### Gleason Scores

In some embodiments, Gleason scores are used to determine whether a cancer is a serious cancer. In some embodiments, High Gleason scores are used as a main focus for determining cancer screening effectiveness. In some embodiments, Gleason scores are used to evaluate sensitivity to serious cancers. In some embodiments, Gleason scores are used to evaluate effectiveness of the screening methods described herein. However, Gleason scores are not the only method for determining cancer screening effectiveness. In some embodiments, Gleason scores are used in combination with other ways of classifying cancers known in the art or described herein.

The Gleason Grading system is used to help evaluate the prognosis of men with prostate cancer. Together with other parameters, it is incorporated into a strategy of prostate cancer staging which predicts prognosis and helps guide therapy. A Gleason score is given to prostate cancer based upon its microscopic appearance. Cancers with a higher Gleason score are more aggressive and have a worse prognosis. Recurrence rates are also much higher for high Gleason cancers than low Gleason cancers.

Studies have shown that the risk of recurrence after radical prostatectomy (RP) increases steeply as Gleason score increases and increases as clinical stage increases. For example, results from Johns Hopkins have shown that for a PSA in the 4-10 range at stage T2a, the risk of recurrence at ten years is over 5 times greater for Gleason 8-10 (37%) than for Gleason 6 (7%). (See Han et al., Journal of Urology 2003)

The pathologist assigns a grade to the most common tumor pattern, and a second grade to the next most common tumor pattern. The two grades are added together to get a Gleason score. For example, if the most common tumor pattern was grade 3, and the next most common tumor pattern was grade 4, the Gleason score would be 3+4=7. The Gleason grade is also known as the Gleason pattern, and the Gleason score is also known as the Gleason sum. The Gleason grade ranges from 1 to 5, with 5 having the worst prognosis. The Gleason score ranges from 2 to 10, with 10 having the worst prognosis. For Gleason score 7, a Gleason 4+3 is a more aggressive cancer than a Gleason 3+4. There is not much difference between the aggressiveness of a Gleason score 9 or 10 tumor.

Gleason scores are associated with the following features:

Grade 1—The cancerous prostate closely resembles normal prostate tissue. The glands are small, well-formed, and closely packed.

Grade 2—The tissue still has well-formed glands, but they are larger and have more tissue between them.

Grade 3—The tissue still has recognizable glands, but the cells are darker. At high magnification, some of these cells have left the glands and are beginning to invade the surrounding tissue.

Grade 4—The tissue has few recognizable glands. Many cells are invading the surrounding tissue

Grade 5—The tissue does not have recognizable glands. There are often just sheets of cells throughout the surrounding tissue.

In some embodiments, a high Gleason score is considered to be 4+3 or 8-10. These high Gleason scores are not considered indolent because of their Gleason score and because ones found and treated are seldom small. Moreover, studies have shown a sharp increase in recurrence risk from Gleason 3+4 (7) cancers, which behave more like Gleason 6 (usually 3+3) cancers, to Gleason 4+3 (7) cancers.

### Screening Size Cancers

In some embodiments the methods of the invention are directed towards detecting cancers large enough to be found with reasonable reliability using PSA based screening methods. In some embodiments, the size of the cancer is used to determine the progression or aggressiveness of the cancer. In some embodiments, analysis according to the invention uses a tumor volume threshold of 1 cc and/or a roughly corresponding clinical pathology stage of T2b or greater when tumor volume is not available. As one non-limiting example, analysis suggests that median PSA tumor density is about 1.9, which means a typical man with a 1 cc tumor and no BPH or prostatitis will have a PSA of about 2.9 (1.9 PSA from cancer and roughly 1.0 baseline PSA from the prostate). For a PSA of about 2.9, a low risk of recurrence is typically observed, roughly in the 5% to 7% range. In another non-limiting example, a tumor volume of 0.5 cc typically has a PSA of about 1.8 (0.8 PSA from cancer and roughly 1.0 baseline PSA from the prostate). The Hopkins work suggests a PSA this low might have a recurrence risk in the 3% to 4% range, or only 2% to 3% lower than PSA at 2.9. In some embodiments, tumor volumes below 0.5 cc are thought to produce too little PSA to be reliably detected by PSA screening methods.

### Systems

In another aspect of the invention, a medical information system for performing an analysis method as described herein is provided that comprises: an input device for receiving subject data; a processor for performing the analysis method; a storage unit in communication with the processor having a database for: (i) storing the subject data; and/or (ii) storing population data related to a biomarker and/or a disease; and an output device that transmits information relating to the probability of a prostate condition to an end user.

The invention also provides a method for assessing a disease in a subject comprising: collecting data from the subject corresponding to a biomarker for the disease at least two different times, wherein the data corresponding to the at least two different times form a trend; exporting said data for manipulation of said data by executing a method of the invention; and importing the results of said manipulation to an end user. For example, data is collected at a first location, such as a hospital, the data is exported to a second location, such as a remote server in any remote location, where a method of the invention is executed to obtain information regarding the disease in a subject, and then the information is imported from the remote location back to the first location, such as the point-of-care in the hospital, or the information is imported to a third location, such as a database.

It is further noted that the systems and methods may be implemented on various types of computer architectures, such as for example on a networked system or in a client-server configuration, or in an application service provider configuration, on a single general purpose computer or workstation. The systems and methods may include data signals conveyed via networks (for example, local area network, wide area network, internet, and combinations thereof), fiber optic medium, carrier waves, wireless networks for communication with one or more data processing devices. The data signals can carry any or all of the data disclosed herein (for example, user input data, the results of the analysis to a user) that is provided to or from a device.

Additionally, the methods and systems described herein may be implemented on many different types of processing devices by program code comprising program instructions that are executable by the device processing subsystem. The software program instructions may include source code, object code, machine code, or any other stored data that is operable to cause a processing system to perform methods described herein.

The systems' and methods' data (for example, biomarker values, fitted trend equations, trend characteristics, associations, mappings) may be stored and implemented in one or more different types of computer-implemented ways, such as different types of storage devices and programming constructs (for example, data stores, RAM, ROM, Flash memory, flat files, databases, programming data structures, programming variables, IF-THEN (or similar type) statement constructs). It is noted that data structures describe formats for use in organizing and storing data in databases, programs, memory, or other computer-readable media for use by a computer program.

The systems and methods may be provided on many different types of computer-readable media including computer storage mechanisms (for example, CD-ROM, diskette, RAM, flash memory, computer's hard drive, including portable hard drive, magnetic tape, and holographic storage) that contain instructions (for example, software) for use in execution by a processor to perform the methods' operations and implement the systems described herein.

The computer components, software modules, functions, data stores and data structures described herein may be connected directly or indirectly to each other in order to allow the flow of data needed for their operations. It is also noted that the meaning of the term module includes but is not limited to a unit of code that performs a software operation, and can be implemented for example as a subroutine unit of code, or as a software function unit of code, or as an object (as in an object-oriented paradigm), or as an applet, or in a computer script language, or as another type of computer code. The software components and/or functionality may be located on a single computer or distributed across multiple computers depending upon the situation at hand.

In general, in yet another aspect, a computer readable medium is provided including computer readable instructions, wherein the computer readable instructions instruct a processor to execute step a) of the methods described above. The instructions can operate in a software runtime environment.

In general, in yet another aspect, a data signal is provided that can be transmitted using a network, wherein the data signal includes said probability or intermediate results calculated in a step of the methods described above. The data signal can further include packetized data that is transmitted through wired or wireless networks.

In an aspect, a computer readable medium comprises computer readable instructions, wherein the instructions when executed carry out a calculation of the probability of a prostate condition in a patient based upon data obtained from the patient corresponding to at least one biomarker. The computer readable instructions can operate in a software runtime environment of the processor. In an embodiment, a software runtime environment provides commonly used functions and facilities required by the software package. Examples of a software runtime environment include, but are not limited to, computer operating systems, virtual machines or distributed operating systems. As will be appreciated by those of ordinary skill in the art, several other examples of runtime environment exist. The computer readable instructions can be packaged and marketed as a software product or part of a software package. For example, the instructions can be packaged with an assay kit for PSA.

The computer readable medium may be a storage unit of the present invention as described herein. It is appreciated by those skilled in the art that computer readable medium can also be any available media that can be accessed by a server, a processor, or a computer. The computer readable medium can be incorporated as part of the computer-based system of the present invention, and can be employed for a computer-based assessment of a prostate condition.

In an embodiment, the calculation of a probability can be carried out on a computer system. The computer system can comprise any or all of the following: a processor, a storage unit, software, firmware, a network communication device, a display, a data input, and a data output. A computer system can be a server. A server can be a central server that communicates over a network to a plurality of input devices and/or a plurality of output devices. A server can comprise at least one storage unit, such as a hard drive or any other device for storing information to be accessed by a processor or external device, wherein the storage unit can comprise one or more databases. In an embodiment, a database can store hundreds to millions of data points corresponding to a biomarker from hundreds to millions of subjects. A storage unit can also store historical data read from an external database or as input by a user. In an embodiment, a storage unit stores data received from an input device that is communicating or has communicated with the server. A storage unit can comprise a plurality of databases. In an embodiment, each of a plurality of databases corresponds to each of a plurality of prostate biomarkers. In another embodiment, each of a plurality of databases corresponds to each of a plurality of possible prostate conditions of a subject. An individual database can also comprise information for a plurality of possible medical conditions or a plurality of biomarkers or both. Further, a computer system can comprise multiple servers.

A processor can access data from a storage unit or from an input device to perform a calculation of an output from the data. A processor can execute software or computer readable instructions as provided by a user, or provided by the computer system or server. The processor may have a means for receiving patient data directly from an input device, a means of storing the subject data in a storage unit, and a means for processing data. The processor may also include a means for receiving instructions from a user or a user interface. The processor may have memory, such as random access memory, as is well known in the art. In one embodiment, an output that is in communication with the processor is provided.

After performing a calculation, a processor can provide the output, such as from a calculation, back to, for example, the input device or storage unit, to another storage unit of the same or different computer system, or to an output device. Output from the processor can be displayed by data display. A data display can be a display screen (for example, a monitor or a screen on a digital device), a print-out, a data signal (for example, a packet), an alarm (for example, a flashing light or a sound), a graphical user interface (for example, a webpage), or a combination of any of the above. In an embodiment, an output is transmitted over a network (for example, a wireless network) to an output device. The output device can be used by a user to receive the output from the data-processing computer system. After an output has been received by a user, the user can determine a course of action, or can carry out a course of action, such as a medical treatment when the user is medical personnel. In an embodiment, an output device is the same device as the input device. Example output devices include, but are not limited to, a telephone, a wireless telephone, a mobile phone, a PDA, a flash memory drive, a light source, a sound generator, a fax machine, a computer, a computer monitor, a printer, an iPod, and a webpage. The user station may be in communication with a printer or a display monitor to output the information processed by the server.

A client-server, relational database architecture can be used in embodiments of the invention. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the invention, the server computer handles all of the database functionality. The client computer can have software that handles all the front-end data management and can also receive data input from users.

In an example, a subject or medical professional enters medical data from a prostate biomarker assay into a webpage. The webpage transmits the data to a computer system or server, wherein the data is stored and processed. For example, the data can be stored in databases the computer systems. Processors in the computer systems can perform calculations comparing the input data to historical data from databases available to the computer systems. The computer systems can then store the output from the calculations in a database and/or communicate the output over a network to an output device, such as a webpage or email. After a user has received an output from the computer system, the user can take a course of medical action according to the output. For example, if the user is a physician and the output is a probability of BPH above a threshold value, the physician can prescribe a pharmaceutical.

In some embodiments, a set of users can use a web browser to enter data from a prostate biomarker assay into a graphical user interface of a webpage. Such a webpage is a graphical user interface associated with a front end server, wherein the front end server can communicate with the user's input device (for example, a computer) and a back end server. The front end server can either comprise or be in communication with a storage device that has a front-end database capable of storing any type of data, for example user account information, user input, and reports to be output to a user. Data from each user (for example, biomarker values and subject profiles) can be then be sent to a back end server capable of manipulating the data to generate a result. For example, the back end server can calculate a probability that a subject has a medical condition using the data input by the user. A back end server can comprise historical data relating to a prostatic condition to be evaluated, or a plurality of prostatic conditions. The back end server can then send the result of the manipulation or calculation back to the front end server where it can be stored in a database or can be used to generate a report. The results can be transmitted from the front end server to an output device (for example, a computer with a web browser) to be delivered to a user. A different user can input the data and receive the data. In an embodiment, results are delivered in a report. In another embodiment, results are delivered directly to an output device that can alert a user.

In an embodiment, a method of the invention comprises obtaining a sample from a subject, wherein the sample contains a biomarker. The sample can be obtained by the subject or by a medical professional. Examples of medical professionals include, but are not limited to, physicians, emergency medical technicians, nurses, first responders, psychologists, medical physics personnel, nurse practitioners, surgeons, dentists, and any other obvious medical professional as would be known to one skilled in the art. The sample can be obtained from any bodily fluid, for example, amniotic fluid, aqueous humor, bile, lymph, breast milk, interstitial fluid, blood, blood plasma, cerumen (earwax), Cowper's fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humour. In an example, the sample is obtained by a blood draw, where the medical professional draws blood from a subject, such as by a syringe. The bodily fluid can then be tested to determine the prevalence of the biomarker. Biological markers, also referred to herein as biomarkers, according to the present invention include without limitation drugs, prodrugs, pharmaceutical agents, drug metabolites, biomarkers such as expressed proteins and cell markers, antibodies, serum proteins, cholesterol, polysaccharides, nucleic acids, biological analytes, biomarker, gene, protein, or hormone, or any combination thereof. At a molecular level, the biomarkers can be polypeptide, glycoprotein, polysaccharide, lipid, nucleic acid, and a combination thereof.

Information can be sent to a computer system automatically by a device that reads or provides the data from a biomarker assay. In another embodiment, information is entered by a user (for example, the subject or medical professional) into a computer system using an input device. The input device can be a personal computer, a mobile phone or other wireless device, or can be the graphical user interface of a webpage. For example, a webpage programmed in JAVA can comprise different input boxes to which text can be added by a user, wherein the string input by the user is then sent to a computer system for processing. The subject may input data in a variety of ways, or using a variety of devices. Data may be automatically obtained and input into a computer from another computer or data entry system. Another method of inputting data to a database is using an input device such as a keyboard, touch screen, trackball, or a mouse for directly entering data into a database.

In another embodiment, a system of the invention can further include a medical test for testing said subject for said prostate condition. The medical test can be a PSA, an FPSA, or a ProPSA assay. In yet another embodiment, a system can further include a medical treatment for treating said subject for said prostate condition.

In an embodiment, a computer system of the invention comprises a storage unit, a processor, and a network communication unit. For example, the computer system can be a personal computer, laptop computer, or a plurality of computers. The computer system can also be a server or a plurality of servers. Computer readable instructions, such as software or firmware, can be stored on a storage unit of the computer system. A storage unit can also comprise at least one database for storing and organizing information received and generated by the computer system. In an embodiment, a database comprises historical data, wherein the historical data can be automatically populated from another database or entered by a user.

In an embodiment, a processor of the computer system accesses at least one of the databases or receives information directly from an input device as a source of information to be processed. The processor can perform a calculation on the information source, for example, performing dynamic screening or a probability calculation method of the invention. After the calculation the processor can transmit the results to a database or directly to an output device. A database for receiving results can be the same as the input database or the historical database. An output device can communicate over a network with a computer system of the invention. The output device can be any device capable delivering processed results to a user. Example output devices include, but are not limited to, a telephone, a wireless telephone, a mobile phone, a PDA, a flash memory drive, a light source, a sound generator, a fax machine, a computer, a tablet, a computer monitor, a printer, an iPOD, and a webpage.

An output of a computer system may assume any form, such as a computer program, webpage, or print-out. Any other suitable representation, picture, depiction or exemplification may be used.

Communication between devices or computer systems of the invention can be any method of digital communication including, for example, over the internet. Network communication can be wireless, ethernet-based, fiber optic, or through fire-wire, USB, or any other connection capable of communication as would be obvious to one skilled in the art. In an embodiment, information transmitted by a system or method of the invention can be encrypted by any method as would be obvious to one skilled in the art. In the field of medicine, or diagnostics, encryption may be necessary to maintain privacy of the data, as well as deter theft of information.

It is further noted that the systems and methods may include data signals conveyed via networks (for example, local area network, wide area network, internet), fiber optic medium, carrier waves, wireless networks for communication with one or more data processing or storage devices. The data signals can carry any or all of the data disclosed herein that is provided to or from a device.

Additionally, the methods and systems described herein may be implemented on many different types of processing devices by program code comprising program instructions that are executable by the device processing subsystem. The software program instructions may include source code, object code, machine code, or any other stored data that is operable to cause a processing system to perform methods described herein. Other implementations may also be used, however, such as firmware or even appropriately designed hardware configured to carry out the methods and systems described herein.

The methods of the invention may be packaged as a computer program product, such as the expression of an organized set of instructions in the form of natural or programming language statements that is contained on a physical media of any nature (for example, written, electronic, magnetic, optical or otherwise) and that may be used with a computer or other automated data processing system of any nature (but preferably based on digital technology). Such programming language statements, when executed by a computer or data processing system, cause the computer system to act in accordance with the particular content of the statements. Computer program products include without limitation: programs in source and object code and/or test or data libraries embedded in a computer readable medium. Furthermore, the computer program product that enables a computer system or data processing equipment device to act in preselected ways may be provided in a number of forms, including, but not limited to, original source code, assembly code, object code, machine language, encrypted or compressed versions of the foregoing and any and all equivalents.

Information before, after, or during processing can be displayed on any graphical display interface in communication with a computer system (for example, a server). A computer system may be physically separate from the instrument used to obtain values from the subject. In an embodiment, a graphical user interface also may be remote from the computer system, for example, part of a wireless device in communication with the network. In another embodiment, the computer and the instrument are the same device.

An output device or input device of a computer system of the invention can include one or more user devices comprising a graphical user interface comprising interface elements such as buttons, pull down menus, scroll bars, fields for entering text, and the like as are routinely found in graphical user interfaces known in the art. Requests entered on a user interface are transmitted to an application program in the system (such as a Web application). In one embodiment, a user of user device in the system is able to directly access data using an HTML interface provided by Web browsers and Web server of the system.

A graphical user interface may be generated by a graphical user interface code as part of the operating system or server and can be used to input data and/or to display input data. The result of processed data can be displayed in the interface or a different interface, printed on a printer in communication with the system, saved in a memory device, and/or transmitted over a network. A user interface can refer to graphical, textual, or auditory information presented to a user and may also refer to the control sequences used for controlling a program or device, such as keystrokes, movements, or selections. In another example, a user interface may be a touch screen, monitor, keyboard, mouse, or any other item that allows a user to interact with a system of the invention as would be obvious to one skilled in the art.

### EXAMPLES

### Example 1

### Dynamic Analysis

### Materials and Methods

1,038 men from the Tyrol screening project and UCSF and CaPSURE databases were analyzed.

The sources of data were the:
University of California at San Francisco (UCSF) surgery database. Please see: http://www.ucsfhealth.org/clinics/prostate_cancer_center/index.html
Cancer of the Prostate Strategic Urologic Research Endeavor (CaPSURE) surgery database managed by UCSF. Please see: http://urology.ucsf.edu/capsure/overview.htm
Innsbruck Medical University managed surgery database for the Tyrol region of Austria. Please see, for example: Bartsch et. al., Tyrol Prostate Cancer Demonstration Project: early detection, treatment, outcome, incidence and mortality; Urological Oncology, in BJU International, 101, 809-816, 2008.

670 men from the Tyrol screening project had no cancer detected by biopsy and at least 5 PSA tests over 4 years with no gap more than 2 years. These men are referred to as the full history no cancer group. 331 men with no cancer had adequate data up to their last biopsy. These men are referred to as the truncated history no cancer group. 368 men in the University of California at San Francisco (UCSF) and CaPSURE databases and from the Tyrol underwent radial prostatectomy surgery (RP) and had pathological results and the same minimum PSA history. Men with Gleason scores of 4+3 or greater and stage T2b and greater were considered high risk.

The Tyrol Cancer Demonstration Project is a mass prostate cancer screening program in the Tyrol region of Austria started as a demonstration project in 1993. General practitioners, urologists, medical centers, labs and the Tyrol Blood Bank of the Red Cross collaborated in the screening program. Participating volunteers gave informed consent. Men with elevated PSA results, were advised to undergo further urologic exams and treatment, if necessary. For men with normal PSA test results, the protocol was to repeat the PSA test 6-12 month later.

The UCSF database contained men undergoing radical prostatectomy (RP) as a treatment for prostate cancer over several years. CaPSURE is a community database of RP patients managed by UCSF.

TABLE 1
ALL RP (INNSBRUCK, UCSF, AND CAPSURE POPULATIONS)

| Group | N | Innsbruck | UCSF | CaPSURE |
|---|---|---|---|---|
| RP, adequate data | 373 | 143 | 131 | 99 |
| RP, AD, missing pathology | 5 | 5 | 0 | 0 |
| Gleason and/or stage data | | | | |
| High Gleason (4 + 3, 8-10) | 59 | 25 | 22 | 12 |
| Low Gleason (4-6, 3 + 4) | 309 | 113 | 109 | 87 |
| Low Gleason, Low Stage | 63 | 21 | 21 | 21 |
| (T1a-c,T2a) | | | | |
| Low Gleason, High Stage | 246 | 92 | 88 | 66 |
| (T2b-c, T3a-c, T4) | | | | |
| Recur | 32 | 16 | 7 | 9 |
| Innsbruck No Cancer, Adequate | 331 | | | |
| Data (up to most recent biopsy) | | | | |
| Innsbruck No Cancer, Adequate | 670 | | | |
| Data (full PSA history) | | | | |

Consistent exponential PSA trends were fit for every man. The functional form included a constant to represent unchanging (or slowly changing) no cancer PSA plus an exponential function to represent the accelerating growth in PSA from progressing cancer. Iterative weighted least squares methods were used to estimate the parameters of the function. An iterative process was used to converge on a consistent trend where all tests included in the estimation of the trend were within 20% of the trend at the time of the test.

Trend PSA (trPSA) was calculated as the value of the trend at the time of the last PSA test. trPSAV was calculated as the slope of the exponential trend at the time of the last PSA test. trPSA from cancer, trPSA(PCa), was calculated as trPSA minus the constant in the functional form (which is a measure of the PSA not contributed by progressing cancer). Estimated growth rate in cancer PSA, PSAgr, was calculated as trPSAV/trPSA(PCa). The same methods were used for men with no cancer.

PSA variation (PSAvar) is a discounted estimate of percentage variation around the consistent trend. It resembles a coefficient of variation where the past is discounted in order to emphasize recent results. 40% was used in this analysis.

A single PSA result was used as the indicator of static PSA screening: either the last PSA test recorded or the last test before biopsy.

High-risk cancers were defined as Gleason scores of 4+3 and above and stage T2b and greater.

Risk assessment was performed using receiver operating characteristic curves (ROC). The threshold PSA was varied for static PSA screening and values for sensitivity to high-risk cancers and specificity to no cancer were calculated. Both full history and truncated history no cancer groups were evaluated. For Dynamic Analysis, a single quadratic parameter (q) was used to define a threshold for PSAgr and PSAvar (PSAvar=q*PSAgr̂2). Combinations were considered to be above the threshold for ROC purposes if PSAgr was above the threshold and PSAvar was less than the threshold.

Additional risk assessment was performed using the percentage of high PSAgr (>15%) cancers missed at a given sensitivity. 15% PSAgr was chosen because it is an integer PSAgr roughly half way between the mean PSAgr for men who died from prostate cancer in Carter's article (20% from our analysis of the data in the article) and the mean PSAgr for men with prostate cancer who did not die from it (11%). For the threshold underlying each sensitivity for a screening method, the percentage of high PSAgr cancers was determined that would have remained undetected by that threshold.

### Results

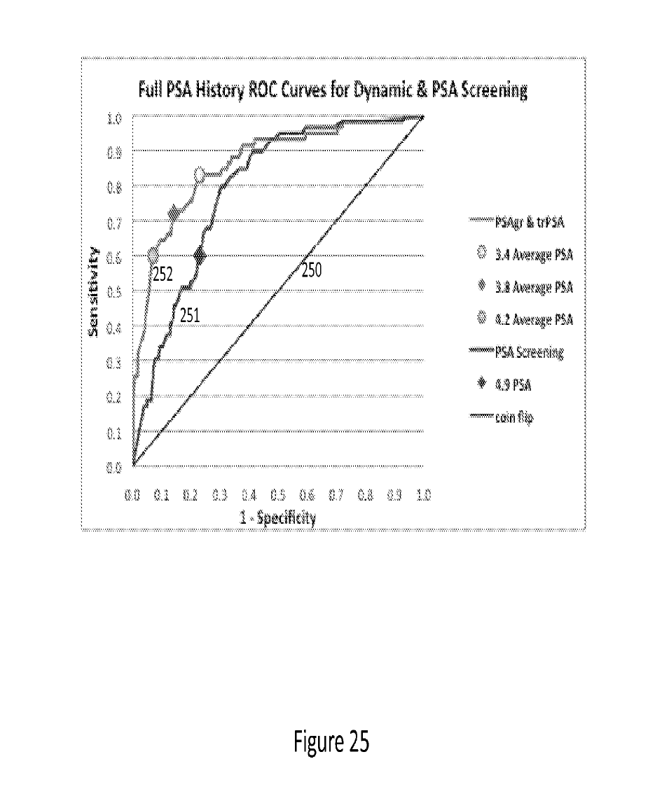

Results are depicted in FIG. 25. The AUC increases to 0.86 for Dynamic Analysis using PSAgr and PSAvar from 0.79 for a static PSA threshold for full PSA history. Dynamic Analysis offers men and their doctors the opportunity to increase sensitivity to serious cancers or increase specificity to no cancer or a preferred combination of both. The following range of improvements are shown on FIG. 25 for full PSA history:
93% specificity instead of 77% at 60% sensitivity.
83% sensitivity instead of 60% at 77% specificity.
86% specificity and 72% sensitivity instead of 77% and 60%.

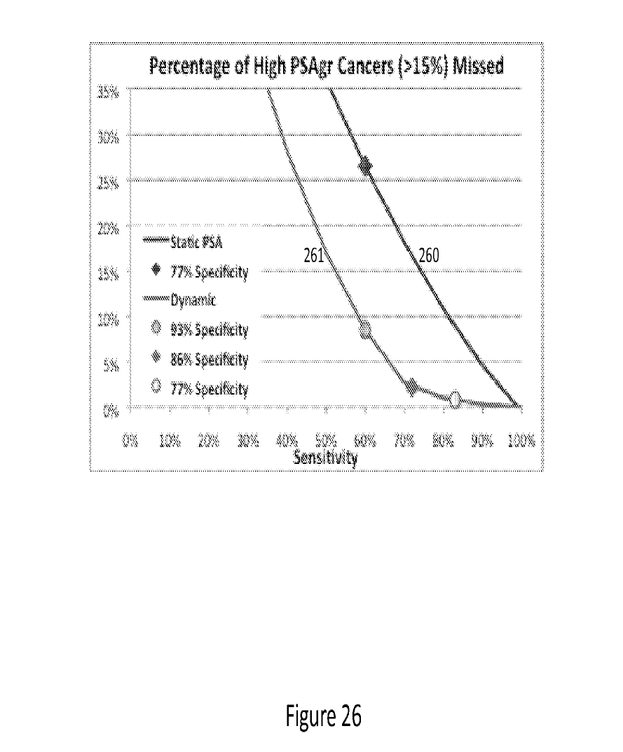

For any sensitivity to serious cancers, Dynamic Analysis misses a lower percentage of high PSAgr cancers than does static PSA screening. The following range of alternatives are shown in FIG. 26 for a full PSA history:
Static PSA Screening:
   27% missed at 60% sensitivity and 77% specificity.
Static PSA Screening:
   8% missed at 60% sensitivity and 93% specificity.
   2% missed at 72% sensitivity and 86% specificity.
   1% missed at 83% sensitivity and 77% specificity.

### Discussion

Dynamic Analysis using PSAgr and PSAvar offers patients and doctors an improved range of choices for detecting high-risk cancers and distinguishing them from no cancer (AUC of 0.86 compared to 0.79 for static PSA screening for full PSA history). For example, specificity can be increased from 77% to 93% or sensitivity can be increased from 60% to 83% or some combination of increases.

Recent work has revealed that the proportion of high Gleason cancer increases for increasing PSAgr: for example, only 10% of cancers are high Gleason for low PSAgr from 0% to 10% compared to 38% for high PSAgr from 30% to 50%. Reevaluation of Carter's work shows that estimated average PSAgr is 20% for men who died of prostate cancer, 12% for men with no cancer and 11% for men who did not die from prostate cancer. These results raise the question of shifting the dominant screening focus from PSA only toward one that considers PSAgr more heavily because of its relationship with high Gleason cancers and cancers that are deadly.

### Conclusion

Dynamic analysis using PSAgr and PSAvar can help improve sensitivity to the most serious cancers and specificity to no cancer found by biopsy. In addition, Dynamic Analysis misses a lower proportion of high PSAgr cancers, that may pose higher risks of death.

### Example 2

### Using FPSA to Distinguish Between Different Prostate Conditions

A simulator was built of a large population of men based on the published evidence about the distribution of cancer, its static and dynamic characteristics, and the results of antibiotic and anti-inflammatory treatment.

Healthy prostate, cancer, BPH, prostatitis due to infection and prostatitis due to inflammation were distinguished using static, dynamic and/or differential analysis of FPSA and PSA (FPSA %, FPSAV %, and FPSAΔ %, respectively). FPSA and PSA values were measured using Beckman-Coulter Access Immunoassays, which are used for FDA approval for the use of FPSA and FPSA % for prostate cancer screening in conjunction with PSA. FPSA % at various time points were calculated as a ratio of FPSA and PSA. FPSAV and PSAV were calculated based on the slope of a fitted trend at the time of the last test as determined by Dynamic Analysis using a constant+exponential functional form. FPSAV % was calculated as a ratio of FPSAV and PSAV. FPSAΔ and PSAΔ were calculated as the change in FPSA and PSA, respectively, before and after differential treatment using antibiotics and anti-inflammatory medications.

The observed values of FPSA %, FPSAV %, and FPSAΔ % for each prostate condition are listed below.
FPSA %:
   Healthy prostate: 29% on average over time
   Cancer: decreases to 20% and 10% as cancer progresses.
   BPH: increases very slowly to 30% and more.
   Inflammation: little change, remaining at an average of about 29%
   Infection: typically large decrease to 10%-15% or less.
FPSAV %:
   Healthy prostate: N/A, because of no significant change in PSA or FPSA (PSAV˜0).
   Cancer: very low, roughly 6%.
   BPH: high, over 30%.
   Inflammation: little change from FPSA % at 29%.
   Infection: typically very low 0% to 10% or even negative.
FPSAΔ %:
   Healthy prostate: N/A because of no significant change in PSA or FPSA.
   Cancer: N/A because of no significant change in PSA or FPSA.
   BPH: N/A because of no significant change in PSA or FPSA.
   Inflammation: little change from FPSA % at 29%.
   Infection: typically very low; 0% to 10% or even negative.

As shown above, FPSA %, FPSV % and FPSAΔ % can distinguish between progressing cancer from healthy prostate, BPH and prostatitis due to inflammation, but cannot always distinguish between progressing cancer and prostatitis due to infection.

### Infection is the Main Cause of Low FPSA % at High PSAs in U.S.

Analysis of CDC data shows that, in subjects with prostatitis due to infection, median FPSA % drops from roughly 29% at a PSA of 1.0 to roughly 10% at a PSA of 9.0—which looks a lot like progressing cancer. Studies have shown that prostate infections can reduce FPSA % dramatically as PSA caused by infection increases. Studies have also shown that, on average, BPH increases FPSA % slowly as prostates grow. A variety of evidence suggests that prostatitis due to inflammation without infection increases PSA with relatively little change in FPSA %. Therefore, infection is believed to be the primary non-cancer cause of reduced FPSA % when PSA increases. Thus, differential treatment with antibiotics and anti-inflammatory medication will increase in effectiveness as PSA increases and the proportion of infection increases. Also, differential treatment will increase substantially in effectiveness at lower FPSA % when the proportion of infection is much higher, even at lower PSAs.

Dynamic Analysis—Greater than 84% AUC Using PSA Only

In some embodiments, Dynamic Analysis can also improve AUC using FPSA. However, the increase may not be dramatic if infection is the primary cause of both high PSAgr and low FPSA %/FPSAV %. If the correlation is high then the improvement of adding FPSA to Dynamic Analysis only may be limited. This will be studied using a new retrospective study of Dynamic Analysis using FPSA and FPSA trends.

### Example 3

### Dynamic Analysis

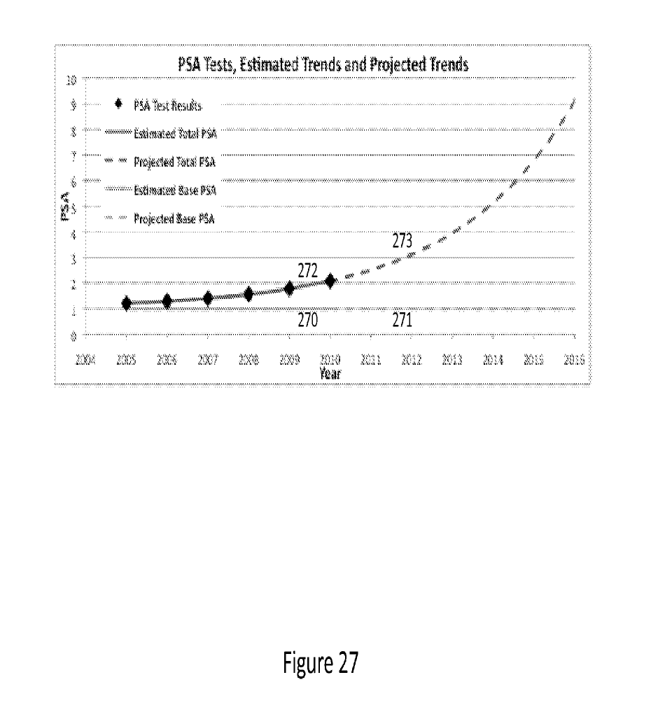

Estimated and Projected PSA Trends—FIG. 27 shows six calibrated and adjusted PSA test results for an example subject over the last five years. A total consistent exponential plus constant PSA trend (272) is estimated using iterative methods to eliminate inconsistent results from the estimation process. An exponential plus constant function is used because PSA from progressing cancer usually grows exponentially and a main interest lies in detecting progressing cancer. The estimated trend is projected into the future (273). Base PSA is estimated (270) as part of the trend estimation process. It is assumed that base PSA (PSAn) is constant or grows very slowly. The estimation process treats it as a constant and projects it into the future at the constant level (271). If cancer is progressing then the difference between total PSA and base PSA is an estimate of PSA from cancer (PSAc). It is used to estimate cancer PSA growth rate (PSAgr=PSAV/PSAc), an important variable for the subject analysis. It affects the risk of unwarranted biopsy (UB %) and recurrence. Other variables are calculated, including PSA variation (PSAvar) and PSA acceleration (PSAacc). They affect the risk of unwarranted biopsy.

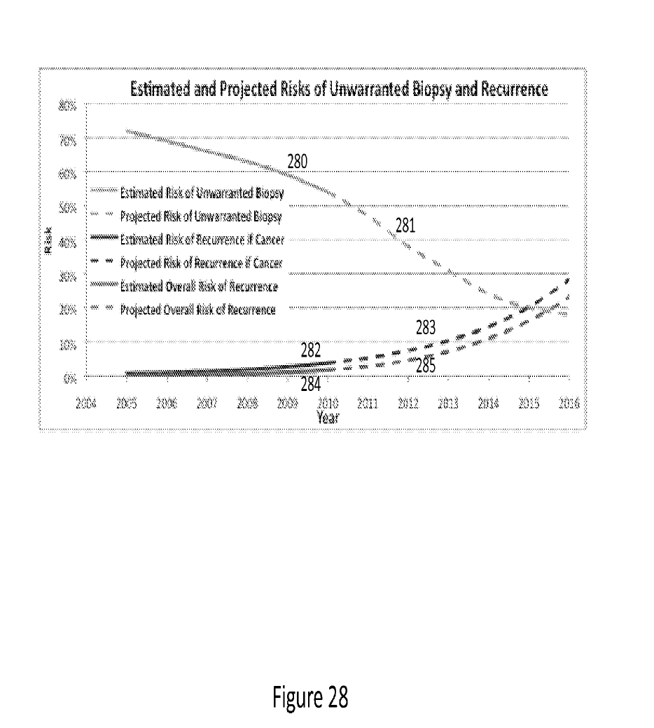

Estimated and Projected Risks—FIG. 28 shows estimated and projected risks of unwarranted biopsy (280, 281), recurrence if cancer (282, 283) and overall recurrence (284, 285). The projections are a “what if” scenario that considers the possibility that the PSA trends continue (and are caused by cancer for the curve showing the risk of recurrence if cancer). The patient and doctor can choose a biopsy now with a high risk of unwarranted biopsy and low risks of recurrence or wait. In a likely scenario, concerns about progressing cancer will weaken if future PSA tests fall below the projected trend or nearly disappear if PSA decreases below recent levels. In a less likely scenario, progressing cancer is the cause of increasing PSA. In this scenario, waiting for stronger evidence of progressing cancer will decrease the risk of unwarranted biopsy at the cost of increased overall risk of recurrence and increased risk of recurrence if cancer. The overall risk of recurrence reflects a “real” cost of waiting that takes into account the probability that a biopsy will find cancer, as well as the risk of recurrence if cancer is the cause.

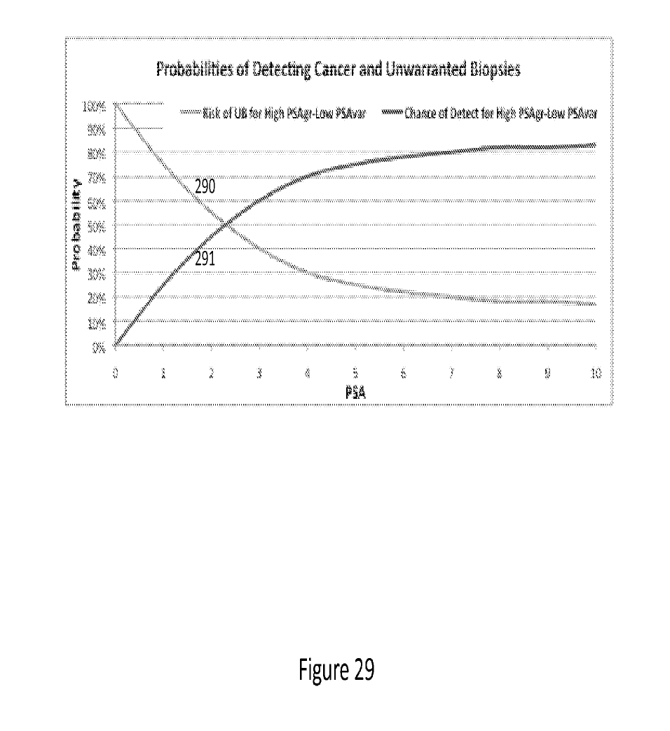

Probability of Detecting Cancer and Unwarranted Biopsies—The probability that a biopsy will find cancer is one minus the risk of unwarranted biopsy that does not find cancer. FIG. 29 shows these probabilities for UB % (290) and for Find Cancer % (291) for an example where PSAgr is high (perhaps 40%), PSAvar is low (perhaps 8%) and PSAaccel is high. The probability of detecting cancer increases for:
Higher trend PSA (and PSAV) but with diminishing returns.
Higher PSAgr
Higher PSAacc
Lower PSAvar

Recurrence Risks—The overall risk of recurrence reflects a “real” cost of waiting that takes into account the probability that a biopsy will find cancer, as well as the risk of recurrence if cancer. The risk of recurrence if cancer increases with:
Higher PSA (implicitly from cancer)
Higher PSAgr (which increases the probability of high Gleason score)
Small prostate volume

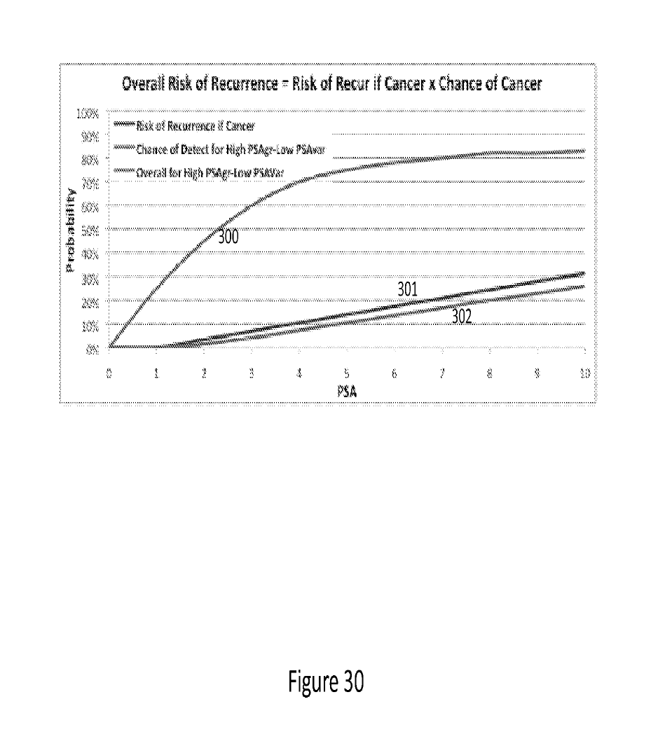

The overall risk of recurrence is the risk of recurrence if cancer times the probability that cancer is detected by biopsy. FIG. 30 shows the relationship among the chance of detecting cancer, the risk of recurrence if cancer and the overall risk of cancer. The top curve (300) shows the chance of detecting cancer for a PSA trend with high PSAgr and low PSAvar that increases as PSA increases. The middle curve (301) shows the risk of recurrence if cancer is detected that increases slowly as PSA increases. The bottom curve (302) shows the overall risk of recurrence that is the product of the chance of detection and the risk of recurrence if detected.

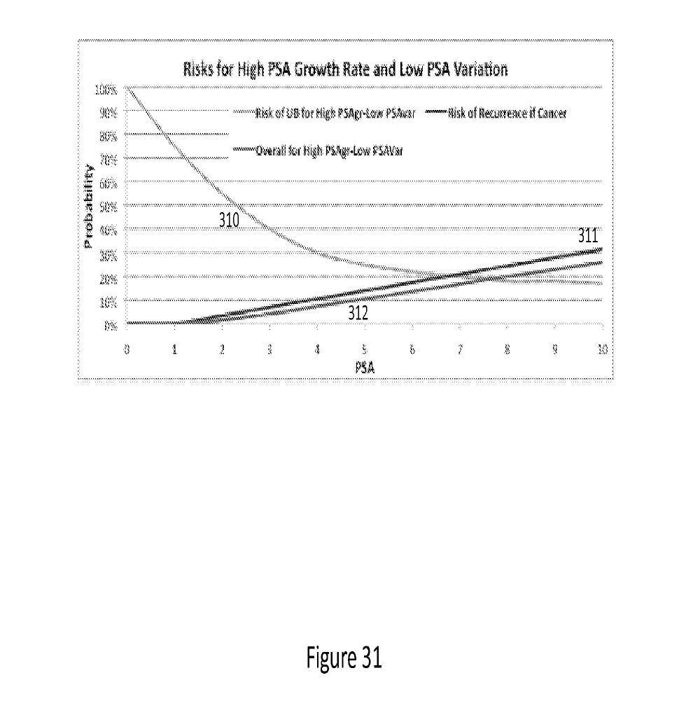

Risks of Unwarranted Biopsy and Recurrence—FIG. 31 presents these risks together vs PSA: Risk of UB for High PSAgr-Low PSAvar (310), Risk of Recurrence if Cancer (311) and Overall (Risk) for High PSAgr-Low PSAvar (312). The curves are used to convert estimated and projected PSA trends to risks plotted vs year, as shown elsewhere herein.

### Example 4

### Calculating Variation

### 20% Variation Around a Linear Trend

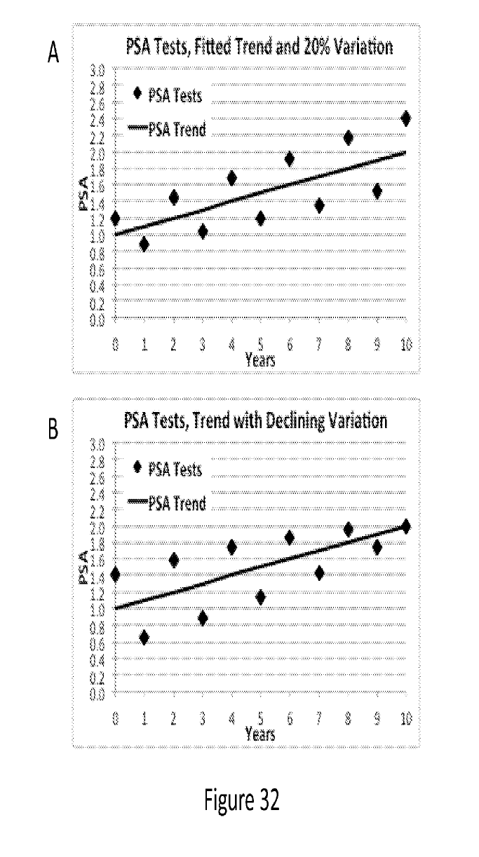

Consider an example where PSA varies by 20% around an increasing linear trend, as shown in FIG. 32A.

### Equal Weights for 20% Variation

First, we will consider equal weights for each PSA value, where all W(t)=1.0. The four example measures of PSA variation around the trend are: average absolute differences from the trend (0.30), average absolute percentage differences from the trend (20%), standard deviation of differences from the trend (0.32) and standard deviation of percentage differences from the trend (21%).

### Discounted Weights for 20% Variation

Second, we will consider discounted weights, where the weight for each previous year is discounted by 40% and they sum to the number of tests (11)—or equivalently: W(t)=0.1114*(1+40%)̂t. The four example measures of PSA variation around the trend are: average absolute differences from the trend (0.36), average absolute percentage differences from the trend (20%), standard deviation of differences from the trend (0.38) and standard deviation of percentage differences from the trend (21%).

### Declining Variation Around a Linear Trend

Next, consider an example where percentage PSA variation up and down around an increasing linear trend declines over time, as shown in FIG. 32B.

### Equal Weights for Declining Variation

First, we will consider equal weights for each PSA value, where all W(t)=1.0. The four example measures of PSA variation around the trend are: average absolute differences from the trend (0.29), average absolute percentage differences from the trend (22%), standard deviation of differences from the trend (0.33) and standard deviation of percentage differences from the trend (27%).

### Discounted Weights for Declining Variation

Second, we will consider discounted weights, where the weight for each previous year is discounted by 40% and they sum to the number of tests (11)—or equivalently: W(t)=0.1114*(1+40%)̂t. The four example measures of PSA variation around the trend are: average absolute differences from the trend (0.16), average absolute percentage differences from the trend (11%), standard deviation of differences from the trend (0.22) and standard deviation of percentage differences from the trend (15%).

### Example 5

### Differential Analysis

As with Dynamic Analysis, a high PSA alone cannot be relied upon to achieve high specificity because it leads to excessively high risks of recurrence.

Differential Analysis—Much Greater than 75% AUC for PSA Only

A differential analysis using antibiotic treatment along with FPSA % should increase specificity substantially. Studies suggest that an increased PSA and decreased FPSA % is a strong indicator of the presence of progressing cancer or prostate infection—but probably not BPH and/or inflammation. FPSA % improves screening specificity by a limited amount because infection can mimic cancer. However, antibiotic treatment usually reduces or cures infection and reduces PSA while increasing FPSA % (revealing the prior presence of infection).

The Differential Analysis AUC using FPSA % should be much greater than the 75% using only PSA. Typical expected results are as follows:
Low PSA (serious cancer is unlikely):
   High FPSA % (serious cancer is unlikely):
      No or Small Drop % (serious cancer is unlikely):
         Healthy prostate is likely.
Elevated PSA:
   Low FPSA %:
      No or Small Drop %:
         Serious cancer is likely.
      Large Drop % (serious cancer is unlikely):
         Infection is likely.
   High FPSA % (serious cancer is unlikely):
      No or Small Drop % (serious cancer is unlikely):
         BPH is likely and can be confirmed by volume measurement.
      Moderate Drop % (serious cancer is not very likely):
         Inflammation is likely.
      Large Drop %:
         Inflammation is likely (but this case may be unusual).

### Example 6

### Dynamic Differential Analysis

A simulator was built of a large population of men based on the published evidence about the distribution of cancer and its static and dynamic characteristics and the results of antibiotic and anti-inflammatory treatment.

The 88% AUC for Dynamic Differential Analysis is based on simulations. The intuition behind the result is that typical progressing cancer is different from typical BPH and/or prostatitis on many dimensions. Three are considered below:
PSA Growth Rate (PSAgr):
   Cancer: Higher
   Prostatitis: Lower
   BPH: Very low
PSA Variation (PSAvar):
   Cancer: Lower
   Prostatitis: Higher
   BPH: Low but matters little because of very low PSAgr.
PSA Drop % after Antibiotic and Anti-Inflammatory Treatment:
   Cancer: Small
   Prostatitis: Large
   BPH: Low but matters little because of very low PSAgr.

For further confirmation of the Dynamic Differential Analysis methods, a prospective study of 200 men at Innsbruck with 100 men will be performed. A proactive early detection program will be used that systematically looks for men with PSA patterns that have high PSAgr and low PSAvar. These men will be treated with antibiotics and anti-inflammatory meds as part of a differential analysis. The highest risk men will be biopsied immediately. Men with low PSAs will be monitored for months or a year or more to see if PSA continues to increase exponentially.

### Example 7

### Calculating Variation

Weighted variation may be calculated as follows.
1. Given a set of PSA test results and the equation of the best-fit trend for those results, as well as a discount factor for adjusting each result's contribution based on its relative location in time (its weight),
   2. for each test result ‘t’:
      (a) calculate the time into the past as t.Date—mostRecentTestDate
      (b) calculate the discount factor as (1.0+discount rate)̂(time into past)
      (c) use the discount factor to calculate an integer weight for t's deviation, as weight=round(10,000*discount factor)
      (d) add the weight to a cumulative total weight
      (e) calculate the deviation from the trend as t.Value—trend value at t
      (f) add the deviation to a cumulative total deviation.
3. Calculate the mean deviation as (cumulative total deviation/number of test results).
4. Calculate the weighted standard deviation of deviations as follows: for each test result ‘t’:
   (a) square the difference between t's deviation and the mean deviation
   (b) multiply this square by t's time-based integer weight
   (c) add this product to a cumulative sum.
5. Divide the cumulative sum-of-weighted-squares sum by the cumulative total weight.
6. Take the square root of the quotient.

### Example 8

### Testing Dynamic Differential Analysis on a Large Data Set

The purpose of the study described in this example is to validate (or refute) Dynamic Differential Analysis (Dynamic Screening) and to evolve and improve it using the Veteran Administration's large population of men.

There are two major aspects of our study: I) Data Acquisition, Cleaning and Processing, and II) Analysis. We will devote substantial amounts of time and effort to acquiring and cleaning the data, including creating new consistent PSA histories. Dynamic Screening software will be used to estimate consistent PSA trends. Our extensive, automated analysis will initially focus on the relationship between the most dangerous cancers and estimated Dynamic Screening parameters. Multi-variable predictive response surfaces will be estimated. Ultimately, the results will be transformed into algorithms in a Clinical Decision Support System.

We expect to find that Dynamic Screening is a superior way to screen for prostate cancer. Specific expected findings include: 1) deadly (and dangerous) cancers typically produce smooth exponential growth in PSA above a baseline, 2) the faster the growth the more deadly the cancer, and 3) increased PSA test variability around a trend indicates a greater chance of prostatitis and/or BPH and a smaller chance of deadly cancer.

Dynamic Screening uses algorithms to fit consistent trends to a man's PSA history. Consistent trends exclude anomalous jumps that are likely to have been caused by prostatitis and strongly consider lower bound tests that are most likely to reflect an underlying source of increasing PSA from cancer. The functional form consists of a no cancer baseline, PSAn, plus an exponential function that may reflect increasing PSA from cancer, PSAc. PSAc has a growth rate, PSAgr, that tends to reflect the deadliness of the cancer. The higher the growth rate the more deadly the cancer—if progressing cancer is the cause of the increasing PSA. In addition, PSA variability is measured in several ways. PSA from deadly cancers tends to grow exponentially in a smooth curve with little variation, while PSA from other causes may not grow exponentially and often varies around the trend, sometimes with jumps and drops. Personalized simulations are used to evaluate the benefits of delay to gather more information vs biopsy and treatment now.

There are two major aspects of our study: I) Data Acquisition, Cleaning and Processing, and II) Analysis.

### I. Data Acquisition, Cleaning and Processing

There are four parts of the data aspect: 1) Use the VA Informatics and Computing Infrastructure (VINCI), 2) Acquire and Clean Data Iteratively, 3) Clean Data for Consistency and 4) Process Data.

### I.1. Use VINCI Primarily

VINCI will host our primary data storage and analysis workspace. However, some preliminary data acquisition and analysis may start on other secure VA servers. No primary data will be printed or transferred to personal computers or laptops.

I.1.a. Primary Use of VINCI

Most data will be stored in a secure workspace on a VINCI server, and all analysis will be performed in that workspace. The VA Informatics and Computing Infrastructure (VINCI) is an initiative to improve researchers' access to VA data and to facilitate the analysis of those data while ensuring Veterans' privacy and data security. Researchers will access the data along with the tools for reporting and analysis in a secure Workspace. VINCI has a common access point using Remote Desktop Connection to connect from anywhere within the VA network. Files required to be moved from the VINCI environment to a researcher's PC (located on the VA Network outside of VINCI) will be possible through a monitored, audited, and controlled data transfer environment. This area will provide a location where VINCI can allow data to be moved for decision support while preventing the removal of sensitive information. Our researchers will access VINCI and the VA data available through it from computers at VASNHCS offices and from computers with VA approved VPN access.

I.1.b. Secondary Use of Other Secure VA Servers

In our early exploration process, it may be necessary to examine and analyze data on the secure VA server which contains the data. We will access this data under the auspices of the appropriate VA data steward. If useful, we will then request transfer of the data to the VINCI system.

### I.2. Acquire and Clean Data Iteratively

Once authorization is received from the VA stewards for specific data, the VINCI staff will arrange for the transfer of the data to our workspace. The VA data necessary for this study varies greatly in terms of availability, approvals and issues (how clean is the data). Therefore, we will proceed to acquire data in an iterative fashion starting with the cleanest data that is easiest to obtain and easiest to receive approval—and then work our way through more difficult and problematic data. VA data systems are complex, and we cannot anticipate all sources of valuable data that we may discover. Therefore, the data sources discussed below are the biggest and most well-known but not necessarily the only ones we will use.

I.2.a. Demographic and Physical Data

We will use a limited amount of demographic data, including age and race, and physical data, including height and weight (to calculate Body Mass Index). The data resides in: VA Integrated Service Networks (VISNs) Data Warehouses (VDWs) and the national VA Corporate Data Warehouse (CDW).

I.2.b. PSA Histories

PSA histories are a key element of our study. The data resides in: VA Integrated Service Networks (VISNs) Data Warehouses (VDWs), the national VA Corporate Data Warehouse (CDW) and specialized data bases, such as the VA Decision Support System (DSS). Our focus will be on men with multiple PSA tests.

The VA started administering PSA tests and collecting data in the early 1990s—over 20 years for some men. However, relatively clean PSA data in VDWs and the CDW may only exist for the last seven years. Longer PSA histories exist, perhaps in CDW and most likely in DSS. However, this data must be cleaned before use. For example, each VISN and probably each medical center chose their own “code” for PSA with up to forty variations (PSA, psa, prostate specific antigen, etc.). Up to now, little or no systematic effort has been made to clean up this data and restructure it in a consistent way suitable for analysis. We plan to clean up as much of this data as we can for use in our study and for subsequent use by other VA researchers. Key steps will include identifying all codes used for PSA, searching for those codes in all relevant fields and converting those codes to a consistent code suitable for analysis, such as PSA.

I.2.c. Digital Rectal Exams

Results of digital rectal exams (DRE) have been used as an indicator of possible prostate cancer. We suspect that DRE results data may reside in: VA Integrated Service Networks (VISNs) Data Warehouses (VDWs) and the national VA Corporate Data Warehouse (CDW). There is some chance that we may have to look for results in notes. If so, we will scale back this effort to a small representative sample.

I.2.d. Biopsy and TURP Date and Pathology

Prostate cancer can be diagnosed by needle biopsy, BPH treatment that removes prostate tissue (such as a Transurethral Resection of the Prostate, or TURP) or indicators of advanced cancer (such as a positive bone scan). We will use records of biopsy and TURP pathology. They can be valuable indicators of deadly cancer. Pathology variables include Gleason score, stage, seminal vesicle invasion, prostate volume (if available) and any other key variables contained in the records. Initially, we plan to explore records at the medical center level and the VISN level to understand the data issues. Ultimately, we will use data from Surgical Pathology and request pathology records from the VA Central Cancer Registry (VACCR).

I.2.e. Prostate Cancer Treatment and Date

We will use records of prostate cancer treatment, including: surgery of various kinds, radiation of various kinds and hormone therapy of various kinds. We expect to find these records in VDWs and the CDW.

### I.2.f Prostate Cancer Event History

We will consider applying for prostate cancer event history from the Critical Case Registry (CCR) for some of the categories listed below.

I.2.g. Surgery Date and Pathology

We will use records of surgery pathology. They can be valuable indicators of deadly cancer. Pathology variables include Gleason score, stage including extra-capsular extension, seminal vesicle invasion, prostate weight/volume and any other key variables contained in the records. Initially we plan to explore records at the medical center level and the VISN level to understand the data issues. Ultimately, we will use data from Surgical Report and/or Surgical Pathology and request pathology records from the VA Central Cancer Registry (VACCR). We plan to depend on VACCR and surgical pathology data as much as possible. Our fallback position is to manually (visually) inspect the notes in Surgical Pathology for surgeries prior to VACCR's Jan. 1, 2004 start date with no data in Surgical Pathology for the remainder of the perhaps 5,000 to 10,000 VA men treated with surgery who have the most complete pre-treatment PSA history. We believe that we can quickly identify and record the few key variables of greatest interest, such as: Gleason score (x+y), stage including extra-capsular extension and prostate weight.

I.2.h. Prostate Cancer Recurrence

Prostate cancer recurrence is only well-defined after surgery (and not as well-defined after radiation treatment and not well-defined at all after only hormone therapy) because initially successful surgery reduces PSA to (virtually) undetectable levels. Therefore, we will focus on recurrence only after surgery. We will use standard indicators of biochemical recurrence, such as a confirmed increase in post-treatment PSA to 0.2 or above. In addition, we will look for other indicators of recurrence diagnosis, such as an appropriate diagnosis code, salvage treatment (follow-up surgery or radiation) or the start of hormone therapy. We will use time to recurrence after treatment and PSA doubling time at recurrence as indicators of the severity of recurrence and the probability of death from prostate cancer. Work at Johns Hopkins has shown that these two parameters (Tr and PSADT) plus pathology Gleason score are strong predictors of prostate cancer-specific death. For the perhaps 5,000 men who recurred after surgery with adequate pre-treatment PSA we will manually (visually) inspect the notes in Surgical Pathology for Gleason score (x+y)- and the few other key variables of greatest interest, such as: stage including extra-capsular extension and prostate weight.

I.2.i. Prostate Cancer Metastasis

We plan a multi-faceted approach to identify the onset of metastatic cancer for the perhaps 5,000 VA men with adequate pre-treatment PSA histories. We hope that for some men there will be a record of prostate cancer metastasis diagnoses in CDW. Our next step will be to look for orders for bone scans to detect metastatic cancer. For these men we will look for other evidence that metastatic cancer was detected, such as: the start of hormone therapy, chemotherapy and palliative treatments typical for men diagnosed with metastatic cancer.

I.2.j. Prostate Cancer Death

The VA has death certificates, and date of death, for all veterans regardless of where they died and that date of death can be found in the Patient Data System. If they died in a VA hospital, we expect to find cause of death. However, many veterans do not die in VA hospitals. We plan to request more comprehensive cause of death data from the Beneficiary Identification Record Locator System (BIRLS), which has cause of death no matter where the veteran died. We would make this request only for men with adequate PSA histories and evidence of sufficiently advanced prostate cancer that could lead to death—metastasis, for example.

I.2.k. Antibiotic and Anti-Inflammatory Treatment

Most increases in PSA are caused by prostatitis and/or prostate enlargement from BPH. Prostatitis is caused by infection and/or inflammation. Treatment for prostatitis with antibiotics and/or anti-inflammatory meds (such as Advil) can reduce PSA when prostatitis is present but may not reduce PSA when prostate cancer is the cause. We will compare records and dates of antibiotic and anti-inflammatory treatment for any reason with changes in PSA trends for men with no evidence of prostate cancer and for men with deadly (or likely to be deadly) prostate cancer.

I.2.l. Prostate Enlargement Treatment

Prostate enlargement caused by BPH can be a serious condition that may be treated by medication or by more invasive measures, such as a TURP. Men treated for BPH may have a measure of prostate volume, which is valuable to our research. Moreover, BPH treatment reduces PSA. We plan to take that effect into account in our analysis.

I.2.m. Lost to Follow-Up

Death or cessation of medical treatment by the VA can account for loss of follow-up. We understand that the VA has death certificates, and date of death, for all veterans regardless of where they died and that date of death can be found in the Patient Data System. We plan to use date of death as an indicator of lost to follow-up. For men of interest (with adequate PSA histories), we will identify indicators of continued treatment by the VA (perhaps orders for medication or treatment of any kind) and use the absence of any of those indicators for more than a one year, for example, as evidence of loss to follow-up.

### I.3. Clean Data for Consistency

Some VA data is very clean and useful as is. However, much of the VA data is not clean enough to use as is. We expect to do an extensive amount of data cleaning. The first phase of data cleaning will be done during the data acquisition process, as described in many of the preceding sections. In addition, we will perform a second level of cleaning, especially for men diagnosed and/or treated for prostate cancer. Our software will inspect our pieced-together history for every man for consistency and completeness. For example, we will review the data for men with an increasing PSA history that drops to virtually undetectable levels but has no record of primary treatment for prostate cancer. In such cases, surgery or intense radiation treatment is likely to have occurred. We will search for evidence of that treatment. Similarly, we will look for evidence of metastasis for men whose post-treatment PSA has increased to very high levels. Looking in the other direction, we will search for evidence of biopsy, diagnosis and primary treatment for men with evidence of metastasis or recurrence but no previous indications of prostate cancer.

### I.4. Process Data

PSA histories are the primary data processed prior to analysis. We will use Dynamic Screening methods to estimate and calculate key variables for pre-treatment PSA history and post-treatment PSA history.

I.4.a. Pre-Treatment PSA History

Pre-treatment PSA history is used to evaluate screening. We will use Dynamic Screening algorithms to fit consistent trends to a man's PSA history. Consistent trends exclude anomalous jumps that are likely to have been caused by prostatitis and strongly consider lower bound tests that are most likely to reflect an underlying source of increasing PSA from cancer. The functional form consists of a no cancer baseline, PSAn, plus an exponential function that may reflect increasing PSA from cancer, PSAc. PSAc has a growth rate, PSAgr, that tends to reflect the deadliness of the cancer. The higher the growth rate the more deadly the cancer—if progressing cancer is the cause of the increasing PSA. In addition, PSA variability is measured in several ways. PSA from deadly cancers tends to grow exponentially in a smooth curve with little variation, while PSA from other causes may not grow exponentially and often varies around the trend, sometimes with jumps and drops. Measures of PSA variability (PSAvar) and Jumps and Drops will be calculated for use in the analysis.

I.4.b. Post-Treatment PSA History

Post-treatment PSA history is used to evaluate the recurrence of prostate cancer after treatment, primarily after surgery. We will use a simple version of Dynamic Screening algorithms to estimate time to (biochemical) recurrence and the trend PSA doubling time (PSADT) and corresponding growth rate. The most deadly cancers are associated with short time to recurrence and short PSA doubling time.

### II. Analysis

There are three parts of the analysis aspect of our study: 1) Mapping of Relationships, 2) Predictive Modeling of Outcomes, 3) Evaluation of Dynamic Screening, and 4) Automation and Mathematics and Statistics. We plan to use a range of standard statistical measures, including p ratios. We expect many of the specific projects to be very high powered because of the large populations: 3 to 4 million men overall, perhaps 1 million men with adequate PSA history—and of those men, at least 100,000 men diagnosed with prostate cancer, roughly 30,000 men treated with surgery, over 6,000 men who recurred after surgery and more than 5,000 men who died of prostate cancer and up to 10,000 men with metastatic cancer. We expect the power to drop toward low levels only when many predictive variables are considered together.

### II.1. Mapping of Relationships

We will map risk measures of deadly and bad cancers versus predictive variables using single and multiple variable methods (response surfaces). Predictive variables include: demographic and physical variables (age, race, BMI, etc.) and Dynamic Screening variables (no cancer baseline PSA, cancer PSA at treatment, exponential growth rate in cancer PSA, variability in PSA around the trend, jumps and drops, response to differential treatment, prostate volume, etc.). Response surfaces will have the general form of, for example:

Risk %=fn(Age,PSAgr,PSAc,PSAvar,PVol,ΔPSAdt,etc.)

We will start with a simple multiple variable linear response surface, for example:

Risk %=a+b*Age+c*PSAgr+d*PSAc+e*PSAvar+f*PVol+g*ΔPSAdt,etc.

For this linear response surface, we will solve for Iso-Risk surfaces (combinations of variables that result in the same Risk %). For example, consider a 5% risk of a bad cancer:

5%=a+b*Age+c*PSAgr+d*PSAc+e*PSAvar+f*PVol+g*ΔPSAdt,etc.

We can then solve for cancer PSA (PSAc) thresholds for 5% Risk % as a function of the other variables:

Threshold PSAc=(5%−[a+b*Age+c*PSAg+e*PSAvar+f*PVol+g*ΔPSAdt,etc.])/d

We plan to repeat this process for non-linear response surfaces, at least in some variables.

II.1.a. Death and Metastasis

We will map the risk of death from prostate cancer, and metastasis if available, for multi-dimensional groups of predictive variables. For example, the risk of death from prostate cancer for four groups based on high and low cancer PSA and high and low growth rate in cancer PSA. Kaplan-Meier and related methods will be used to estimate the cancer specific risk of death over time for each group, or for a multi-variable response surface.

II.1.b. Surgery Recurrence

Deadly cancer must recur before it metastasizes, which must occur before it kills. Research at Johns Hopkins has shown that for cancers that recur after surgery, three recurrence/pathology variables strongly predict the risk of death over time: time to biochemical recurrence, PSA doubling time at recurrence and surgery pathology Gleason score.

We will map the risk of combinations of these variables (high Gleason and short times to recurrence and doubling times) as a function of the predictive variables. We will also map the risk of death over time using the Johns Hopkins results and confirmation of those results using VA data. Please see section II.2 Predictive Modeling of Outcomes below.

We expect the magnitude of the recurrence effect with respect to PSAc and PSAgr to be roughly similar in size to the effect found by the Hopkins researchers for PSA and Gleason score, respectively. Therefore, we are confident that roughly 30,000 men treated with surgery and over 6,000 men who recurred after surgery will provide more than enough power to validate or refute our key hypotheses about Dynamic Screening and prostate cancer recurrence after surgery.

II.1.c. Surgery Pathology

Surgery pathology variables include Gleason score, stage including extra-capsular extension, seminal vesicle invasion, prostate weight/volume and any other key variables contained in the records. We will map the risk of these variables and combinations of these variables as a function of the predictive variables. For example, we will map the combined risk of extra-capsular extension and high Gleason score (4+3 plus 8-10, for example).

II.1.d. Biopsy Pathology

Biopsy pathology variables include Gleason score, stage, seminal vesicle invasion, prostate volume if available and any other key variables contained in the records. We will map the risk of these variables and combinations of these variables as a function of the predictive variables. For example, we will map the combined risk of Stage III/IV and high Gleason score (4+3 plus 8-10, for example).

### II.2. Predictive Modeling of Outcomes

We will predictively model certain outcomes, such as death from prostate cancer, when direct mapping of relationships is not possible or is incomplete. For example, death from prostate cancer often occurs 20 or 30 years (or more) after early detection would have occurred. We will not have long enough PSA histories to capture early detection to death over periods greater than twenty years. We will predict death in two steps that allow us to consider very long time spans. First, we will obtain the map of post-surgery recurrence variables (time to recurrence, PSA doubling time at recurrence and pathology Gleason) vs predictive variables, as discussed previously. Second, we will validate using VA data the results of Johns Hopkins studies that show how to use recurrence data (time to recurrence and PSA doubling time at recurrence) along with pathology Gleason score to predict the risk of death over time.

A study at Johns Hopkins found for 379 men who recurred after surgery (76 men died) that the risks of death after recurrence were statistically significant for all three variables when considered together using multivariable analysis:
P<0.001, <0.001, 0.09 for the three shortest PSADTs vs the longest.
P=0.002 for years to recurrence.
P=0.002 for pathological Gleason score.

We expect the magnitude of the cancer-specific death after recurrence effect with respect to the three variables to be same for the VA population as for the Hopkins population.

We will use the results of the first and second studies to predict the risk of death for every man whose cancer recurred. Third, our study will combine the results of the first and second steps to determine the ability of Dynamic Screening variables to predict the risk of cancer specific death in the future. Dynamic Screening variables are:
Cancer PSA (PSAc)
Growth rate in cancer PSA (PSAgr)
Variability of PSA around trend (PSAvar using several measures)

### II.3. Evaluation of Dynamic Screening

We will evaluate Dynamic Screening vs other screening methods, including a simple PSA threshold, and current VA practice in two ways: area under receiver operating characteristic curves (AUC under ROC curves) and key predicted outcomes, such as death from prostate cancer, number of treatments and number of biopsies.

II.3.a. AUC Under ROC Curves

We will compare Dynamic Screening to other screening methods using receiver operating characteristic curves (ROC curves) and the area under those curves (AUC). ROC curves are a graphical plot of sensitivity to a key variable vs 1 minus the specificity. Typically we will consider the full population of men screened or the population of men biopsied. Sensitivity will be to men with deadly cancers or bad cancers, for example men with surgery pathology extra-capsular extension and high Gleason score of 4+3 or 8-10. ROC curves are attractive because they are well accepted and relatively easy to construct for our study. They are less attractive because they fail to consider the full nature of the screening problem where delay of a biopsy is a critical decision—Is it worth waiting to gain better information to avoid unwarranted biopsies and risk of over-treatment at the cost of increased risk of treatment failure (recurrence) and ultimately death?

II.3.b. Key Predicted Outcomes

We will compare Dynamic Screening to other screening methods using predicted outcomes in order to provide policy decision makers, doctors and men with a deeper understanding of screening tradeoffs. We will use simulation methods when necessary to predict key outcomes for various screening methods, including death from prostate cancer plus the number or risk of biopsies and treatment. We will rely heavily on the results of section II.2., Predictive Modeling of Outcomes, when direct maps of relationships are not available from our analysis.

### Example 9

### Estimating Effectiveness of Dynamic Differential Analysis

We expect Dynamic Differential Analysis to dramatically improve on current practice and on no screening. Our starting point for estimation was US SEER data. Prostate cancer incidence accelerated at the start of the PSA era, peaked and has reached steady state. PSA screening contributed to a 40% increase in U.S. detection of prostate cancer, according to age-adjusted SEER data. Prostate cancer death increased slowly until PSA testing led to a steady decline to 43% of peak in 1992. PSA screening contributed to a 43% decrease in U.S. death from prostate cancer, according to age-adjusted SEER data.

Each year in the U.S., over 800,000 biopsies are performed, roughly 230,000 prostate cancers are detected and roughly 200,000 men are treated while 30,000 men die of prostate cancer. Dynamic Screening has been estimated to achieve:
60% reduction in death from 30,000 to 12,000 per year.
40% reduction in treatment from 200,000 to 120,000 per year.
60% reduction in biopsies from 800,000 to 320,000 (per year).
$6 billion per year in cost savings from less recurrence and death, less over-treatment and fewer unwarranted biopsies.

The process for estimating these benefits is outlined below.

### 1. Seer Baselines

Our first step was to use National Cancer Institute Surveillance Epidemiology and End Results (SEER) prostate cancer incidence and death data to establish a current baseline for: 1) current practice using actual data and 2) for pre-PSA era practice using projections of past SEER rates prior to the impact of PSA screening. Please see the SEER website at: http://seer.cancer.gov/2.

### 2. Improvement on Current Practice Using Surgery Databases

Starting from the SEER current practice baseline, we estimated potential improvement from Dynamic Screening using three databases of men treated with surgery where pathology results and PSA histories were available. The sources of data were the:
University of California at San Francisco (UCSF) surgery database.
Cancer of the Prostate Strategic Urologic Research Endeavor (CaPSURE) surgery database managed by UCSF. Please see: http://urology.ucsf.edu/capsure/overview.htm
Innsbruck Medical University managed surgery database for the Tyrol region of Austria. The vast majority of prostate cancer diagnoses in these databases were triggered by an elevated PSA, and reflect current practice in the U.S.

### 2.a. 40% Reduction in Treatment

For these databases of treatment representative of current screening practices, we found that Dynamic Screening methods would have avoided 40% of the biopsies triggered by elevated PSA and subsequent treatment. Dynamic Screening algorithms reject elevated PSAs as likely false positives through combinations of low exponential trend growth rates above a constant, excessive PSA variability around a trend and other factors. This 40% reduction in treatment is consistent with many estimates that roughly 60% of PSA detected prostate cancers are small, unlikely to be deadly and probably produce very little PSA in the blood. Our results exceeded 40% reduction in treatment for each of the three surgery databases. Therefore, we feel comfortable estimating that Dynamic Screening could reduce current U.S. levels of prostate cancer treatment by roughly 40%.

### 2.b. 60% Reduction in Death

For these databases of treatment representative of current screening practices, we found that Dynamic Screening methods would have avoided 60% of the deaths following prostate cancer recurrence after late treatment. In order for prostate cancer to metastasize and lead to death it must first recur after treatment. Therefore, we focused on men who recurred after surgery in the three databases. We found that most of the recurrence was associated with late treatment measured by high estimates of cancer PSA using Dynamic Screening methods. Work on recurrence after surgery at Johns Hopkins has shown that the risk of recurrence after treatment is nearly proportional to PSA at treatment up to about 15 PSA and roughly proportional at higher levels of PSA.

This means that the risk of recurrence and subsequent death can be reduced by earlier detection at lower levels of PSA. We estimated the cancer PSA at which Dynamic Screening would have detected cancer for each of the men who recurred. We then attenuated the risk of death using the proportional relationship to PSA up to 15 and a reduced relationship above that level. The result was a greater than 60% reduction in death risk from early detection and treatment.

### 3. Improvement on Pre-PSA Era Practice Using BLSA Data

Analysis of Baltimore Longitudinal Study of Aging (BLSA) data produced a reduction in death to roughly the same point as the previous analysis. However, we took an extra step. First, we used analysis of SEER data to estimate U.S. prostate cancer death rates prior to the PSA era. Second, we studied BLSA men who died of prostate cancer, including their PSA level at the time cancer was diagnosed by symptoms or indicators other than PSA. Third, we used Dynamic Screening methods to determine how early their cancer would have been detected—and the estimated level of cancer PSA at the time of detection. Finally, we estimated the reduction in the risk of death from early detection using the methods outlined above. The results are consistent with the previous analysis.

### 4. Reduction in Biopsies Using BLSA Data

We estimated that Dynamic Screening could reduce U.S. biopsies by roughly 60%. At least 75% of U.S. biopsies do not find cancer, and more are unwarranted. Analysis of BLSA men with no prostate cancer detected suggests that Dynamic Screening could reduce by over 90% false positives for men with a PSA of 3 or greater. 90% times 75% equals an estimated 67.5% reduction in biopsies. We get a similar reduction when we analyze Innsbruck (Tyrol, AU) biopsy data.

### Description of the BLSA Analysis

Many of the randomized screening trials use a PSA threshold of roughly 3.0 to trigger consideration of a biopsy. The concern of the US Preventative Services Task Force and others is that use of PSA in this way leads to far too many false positives and biopsies and too much subsequent over-treatment. We considered BLSA men with no cancer detected over their lifetime and focused on men with PSAs that exceeded 3.0 at some time (and might have triggered a biopsy at that threshold).

We found that very simple Dynamic Screening rules could eliminate 93% of the false positives using a simple 3.0 PSA threshold. Our Dynamic PSA Thresholds depend on PSAgr and Surprises (PSA jumps and drops) with a 3.0 threshold used only for the most deadly cancer patterns (smooth exponential growth with a high growth rate—PSAgr greater than 30%). We used the following Dynamic PSA Thresholds to reduce false positives by 93%:

PSAgr=30%+(most deadly cancer growth rates)
3.0 if no surprises (smooth exponential growth increases cancer risk)
5.0 if one or more surprises (jumps/drops reduce cancer risk)

PSAgr from 20% to 30% (moderately deadly cancer growth rates)
4.0 if no surprises
6.0 if one or more surprises

PSAgr from 0% to 20% (least deadly cancer growth rates)
6.0 if no surprises
8.0 if one or more surprises

Thresholds are ideally confirmed by a second test.

We don't contend that these Dynamic PSA Thresholds are ideal. Optimal thresholds need to be much more personalized. However, they do show the potential to reduce false positives and subsequent biopsies and over-treatment by large amounts while catching most deadly cancers very early for effective treatment. Dynamic Screening can reduce false positives so much because most benign increases in PSA vary substantially around the trend and/or grow relatively slowly.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for estimating the probability of a prostate condition in a subject, comprising:
a) obtaining a series of at least a first and a second PSA value from said subject, wherein the PSA values are measured in the subject at at least a first and a second time;
b) performing a dynamic analysis using a computer system, wherein said dynamic analysis comprises fitting said series of PSA values to a functional form equation to form a fitted trend over time and calculating a characteristic of said fitted trend, wherein said characteristic reflects PSA variation from the fitted trend; and
c) estimating the probability of said prostate condition by comparing said PSA variation characteristic with results based on analysis of population data,
wherein a higher PSA variation characteristic indicates a first probability of said prostate condition and a lower PSA variation characteristic indicates a second probability of said prostate condition, wherein the second probability is higher or lower than the first probability.

2. The method of claim 1, wherein said fitted trend comprises a first fitted trend value corresponding to the first PSA value and a second fitted trend value corresponding to the second PSA value,
wherein calculating the PSA variation characteristic comprises calculating a first variation comprising a first variation between the first PSA value and the first fitted trend value, calculating a second variation comprising a difference between the second PSA value and the second fitted trend value, altering the first variation by a first weighting factor, and altering the second variation by a second weighting factor different than the first weighting factor.

3. The method of claim 2, wherein said first PSA value is measured before said second PSA value, and the first weighting factor is second weighting factor.

4. The method of claim 2, wherein performing said dynamic analysis further comprises:
calculating a tolerance range of said fitted trend;
removing a PSA value from said series of PSA values that has a value outside said tolerance range, thereby forming a subseries of PSA values; and
fitting said subseries of PSA values to a functional form equation to form a second fitted trend over time and calculating a characteristic of said second fitted trend; wherein estimating the probability of said prostate condition further comprises comparing said characteristic of said second fitted trend with results based on analysis of population data.

5. The method of claim 1, wherein said fitted trend comprises a first fitted trend value corresponding to the first PSA value and a second fitted trend value corresponding to the second PSA value,
wherein calculating the PSA variation characteristic comprises calculating a first variation comprising a first variation between the first PSA value and the first fitted trend value, calculating a second variation comprising a difference between the second PSA value and the second fitted trend value, altering the first variation by a first weighting factor, and altering the second variation by a second weighting factor is the same as the first weighting factor.

6. The method of claim 1, wherein said prostate condition is selected from the group consisting of: prostatitis, benign prostate hyperplasia, prostate cancer, and no prostate disease.

7. The method of claim 6 wherein the prostate condition comprises prostate cancer.

8. The method of claim 7, wherein the first probability of said prostate condition indicated by the higher PSA variation characteristic comprises a lower probability of prostate cancer.

9. The method of claim 7, wherein the second probability of said prostate condition indicated by the lower PSA variation characteristic comprises a higher probability of prostate cancer.

10. The method of claim 1, wherein said subject is a human.

11. The method of claim 1, wherein said computer system comprises a device for network communication, a storage unit, and a processor.

12. The method of claim 1, wherein the functional form equation takes the form of
PSA(t)=PSAn+M*ê(PSAgr*t), wherein t is the time, PSAn is a baseline PSA value, M is a constant multiplier, and PSAgr is a constant reflecting the exponential growth rate of PSA.

13. The method of claim 12, wherein PSAn is calculated based on analysis of population data.

14. The method of claim 1, wherein obtaining said series of PSA values comprises obtaining at least three PSA values from said subject, wherein the PSA values are measured in the subject at at least three different times.

15. The method of claim 1, wherein the step of calculating a characteristic of said fitted trend comprises:
(d) selecting a target PSA value from said series of PSA values, wherein said target PSA value is measured at a target time;
(e) calculating a trend PSA value based on said functional form equation for said target time; and
(f) calculating the characteristic of said fitted trend, wherein said characteristic reflects a comparison of said trend PSA value and said target PSA value.

16. The method of claim 15, wherein the characteristic of said fitted trend is a difference between said trend PSA value and said target PSA value.

17. The method of claim 15, wherein the characteristic of said fitted trend is the difference between said trend PSA value and said target PSA value, divided by said trend PSA value.

18. The method of claim 1, further comprising:
d) obtaining a third PSA value, wherein said third PSA value is measured in the subject at a third time, wherein said third time is subsequent to said at least first and second times;
e) projecting said fitted trend using said computer system to said third time to calculate a projected PSA value at said third time; and
f) calculating a characteristic of said projected PSA value, wherein said characteristic reflects a comparison of said projected PSA value and said third PSA value, wherein estimating the probability of said prostate condition further comprises comparing said characteristic of said projected PSA value with results based on analysis of population data.

19. The method of claim 18, wherein the characteristic of said projected PSA value is a difference between said projected PSA value and said third PSA value.

20. The method of claim 18, wherein the characteristic of said projected PSA value is the difference between said projected PSA value and said third PSA value, divided by said projected PSA value.
